(19) United States
(12) Patent Application Publication
(10) Pub. No.: US 2024/0026393 A1
(43) Pub. Date: Jan. 25, 2024

(54) EFFICIENT PRODUCTION OF CIS, CIS-MUCONIC ACID FROM MIXED SUBSTRATES OF GLUCOSE, D-XYLOSE AND L-ARABINOSE

(71) Applicants: Alliance for Sustainable Energy, LLC, Golden, CO (US); UT-Battelle, LLC, Oak Ridge, TN (US)

(72) Inventors: Chen LING, Lakewood, CO (US); Christopher W. JOHNSON, Denver, CO (US); Gregg Tyler BECKHAM, Golden, CO (US); George Lee PEABODY, V, Oak Ridge, TN (US); Adam Michael GUSS, Oak Ridge, TN (US)

(21) Appl. No.: 18/123,968

(22) Filed: Mar. 20, 2023

Related U.S. Application Data
(60) Provisional application No. 63/321,332, filed on Mar. 18, 2022.

(51) Int. Cl. C12P 7/44; C12N 9/88; C07C 51/42; C07C 51/36

(52) U.S. Cl. CPC C12P 7/44; C12N 9/88; C12Y 401/01063; C12Y 402/01118; C07C 51/42; C07C 51/36

(57) ABSTRACT

Muconic acid is a molecule that can be converted into direct replacement chemicals for incumbent petrochemicals and performance-advantaged bioproducts. Disclosed herein are Pseudomonas putida KT2440 that are engineered to convert glucose and xylose, the primary carbohydrates in lignocellulosic hydrolysates, to muconic acid using a model-guided strategy to maximize the theoretical yield. Using adaptive laboratory evolution (ALE) and metabolic engineering in a strain engineered to express the D-xylose isomerase pathway, we demonstrated that mutations in the heterologous D-xylose:H+ symporter (XylE), increased expression of a major facilitator superfamily transporter (PP_2569), and overexpression of aroB encoding the native 3-dehydroquinate synthase, enable efficient muconic acid production from glucose and xylose simultaneously. Using the rationally engineered strain, we produced 33.7 g/L muconate at 0.18 g/L/h and a 46% molar yield (92% of the maximum theoretical yield).

### CROSS-REFERENCE TO RELATED APPLICATION

This application claims the benefit of U.S. patent application No. 63/321,332 filed on 18 Mar. 2022 which is incorporated by reference herein its entirety.

### CONTRACTUAL ORIGIN

The United States Government has rights in this invention pursuant to contract no. DE-AC05-00OR22725 between the United States Department of Energy and UT-Battelle, LLC., and pursuant to contract no. DE-AC36-08G028308 between the United States Department of Energy and Alliance for Sustainable Energy, LLC, the Manager and Operator of the National Renewable Energy Laboratory.

### SEQUENCE LISTING

This application contains a Sequence Listing which has been submitted electronically and is hereby incorporated by reference in its entirety. The XML document as filed herewith was originally created on 4 Oct. 2023. The XML document as filed herewith is named NREL 21-104.xml, is 102 kilobytes in size and is submitted with the instant application.

### BACKGROUND

The development of economic processes for the production of biofuels and biochemicals from lignocellulose will be critical to help reduce anthropogenic greenhouse gas emissions associated with fossil fuel consumption. Among the various areas of metabolic space that have been explored for biochemical production, molecules from microbial aromatic catabolic pathways exhibit substantial chemical diversity. Of note, cis,cis-muconic acid (hereafter muconate) is a popular platform chemical from the catechol catabolic pathway that can be produced from lignin-derived aromatic compounds, carbohydrates and waste plastics-derived aromatic compounds. Muconate is a bioprivileged molecule that can be converted into either direct replacement chemicals, such as adipic acid and terephthalic acid, or converted to performance-advantaged bioproducts.

Muconate production from carbohydrates is based on the shikimate pathway for aromatic amino acid biosynthesis and was first demonstrated in recombinant Escherichia coli.5 Erythrose-4-phosphate (E4P) and phosphoenolpyruvate (PEP) are condensed to form 3-deoxy-D-arabinoheptulosonate 7-phosphate (DAHP), which is further converted to 3-dehydroshikimate (3DHS), a key intermediate in the shikimate pathway. From 3DHS, at least 5 pathways have been reported for muconate biosynthesis.25-30 Among these pathways, one proceeds through the intermediate protocatechuate (PCA) via a 3DHS dehydratase (asbF) and results in a higher maximum theoretical yield than the others, which proceed through shikimate via a shikimate dehydrogenase (aroE) (FIG. 1a).

Lignocellulosic biomass represents a vast resource for the production of renewable transportation fuels and chemicals to offset and replace current fossil fuel usage. For many decades, worldwide research efforts have focused on the development of cost-effective processes to selectively convert the polysaccharide components of plant cell walls, namely cellulose and hemicellulose, to fuels and chemicals through biological and chemical pathways. For example, in bioethanol production, biomass typically undergoes a mild thermochemical pretreatment step followed by enzymatic hydrolysis and fermentation to produce ethanol from the monomeric components of both cellulose and hemicellulose.

The lignin component of lignocellulosic biomass is an energy-dense, heterogeneous alkyl-aromatic polymer constructed from phenylpropanoid monomers used by plants for water transport and defense, and it is the second most abundant biopolymer on Earth after cellulose. Lignin is typically underutilized in most selective conversion processes for biofuel production. In the production of fuels and chemicals from biomass, lignin is typically burned for process heat because its inherent heterogeneity and recalcitrance make it difficult to selectively upgrade the monomers to value added products. This limited ability to utilize lignin, despite being the most energy dense polymer in the plant cell wall, is primarily due to its inherent heterogeneity and recalcitrance. Despite having a longer history of use as alternative renewable raw materials, cellulose and hemicellulose still remain important, high volume, readily available renewable raw materials, and next generation technologies that process these polysaccharides efficiently and economically are still needed. Thus, compositions, methods, and processes that can simultaneously and/or in parallel convert all of the substituent components of biomass, e.g. lignin, cellulose, and hemicellulose, to useful chemical intermediates, final chemical products (including fuels), is highly desirable to make steps towards lessening global dependency on petroleum.

However, in order to displace our current petrochemical consumption, an expanded renewable product slate is necessary, similar to the myriad of products currently derived from crude petroleum. This requires efficient and economically viable technology for converting all of the main constituents of biomass, cellulose, hemicellulose, as well as lignin, to useful final products, as well as chemical intermediates that can be converted to useful final products, utilizing either new technologies or existing technologies. The present application provides a suite of innovative technologies that may serve as cornerstones for future biomass-to-chemicals manufacturing plants, wherein these technologies focus on the first task of converting biomass to cis, cis-Muconic acid (hereinafter referred to as “muconic acid”), followed by the second task of converting the muconic acid to useful products including, but not limited to, adipic acid, 1,6-hexanediol, and hydrocarbon fuels.

Genetic engineering of microbial organisms is most commonly known due to the landmark Supreme Court case of Diamond v. Chakrabarty, wherein the court validated Chakrabarty's U.S. Pat. No. 4,259,444, directed to a Pseudomonas putida strain that had been engineered to degrade various oil derivatives, including octane and naphthalene.

Since then, researchers have pursued engineered microorganisms for biologically converting various biomass components to numerous chemical intermediates and products, including muconic acid, followed by conversion to adipic acid. Annual world-wide production of adipic acid in 1989 was estimated at 4.2 billion pounds and production has continued to grow since then. With U.S. production at 1.75 billion pounds in 1992, adipic acid consistently ranks as one the top fifty chemicals produced domestically. Nearly 90% of domestic adipic acid is used to produce nylon-6,6. Other uses for adipic acid include production of lubricants and plasticizers. Thus, there is a large economic driver behind the development of improved methods for muconic acid production, especially for the development of improved production methods that utilize renewable resources.

For example Koppisch et al. (“Koppisch”) describe the use of engineered prokaryotic organisms for converting D-glucose to catechol and muconic acid (WO 2012/106257). This includes the introduction of exogenous decarboxylase genes, including aroY from Klebsiella pneumoniae, and the introduction of exogenous dioxygenase genes for converting catechol to muconic acid, for example catA.

U.S. Pat. No. 5,487,987 to Frost et al. (“Frost”) describes the production of adipic acid through a metabolic pathway producing the cis, cis-muconic acid intermediate, also utilizing D-glucose as the starting material, and Escherichia coli genetically engineered to include genes endogenous to Klebsiella pneumoniae and Acinetobacter calcoaceticus.

Burk et al. (“Burk”) describes the use of engineered microbial microorganisms to produce terephthalate through a muconic acid intermediate comprising trans,trans-muconate and/or cis,trans-muconate, starting with succinyl-CoA as a starting material (WO 2011/017560).

U.S. Pat. No. 8,133,704 to Baynes et al. (“Baynes”) describes the use of genetically engineered microorganisms including E. coli, C. glutanicum, B. flavum, and B. lactofermentum for the eventual production of adipic acid, utilizing carbohydrate starting materials.

Weber et al. describe a genetically modified Saccharomyces cerevisiae to produce cis, cis-muconic acid utilizing aromatic amino acid pathways (Applied and Environmental Microbiology (2012) 78(23), 8421-8430).

Pseudomonas putida has been of particular interest recently, especially since completion of the genomic sequencing of Pseudomonas putida KT2440 (Environmetal Microbiology (2002) 4(12), 799-808). Jimenez et al. have characterized four of the main pathways in the KT2440 strain, including the protocatechuate and catechol branches of the β-ketoadipate pathway, the homogentisate pathway, and the phenylacetate pathway (Environmental Microbiology (2002) 4(12), 824-841).

Even before its genomic sequencing, scientists attempted to use P. putida as an organism for producing muconic acid. For example, U.S. Pat. Nos. 4,480,034 and 4,731,328 describe converting toluene to muconic acid, utilizing engineered microorganisms including Pseudomonas putida.

More recently, Bang et al. (“Bang”) describe the use of a P. putida strain (BM014) for the production of cis, cis-muconic acid utilizing benzoic acid as a starting material (Journal of Fermentation and Bioengineering (1995) 79(4), 381-383). J. van Duuren et al. describe the use of P. putida KT2440 for the production of cis, cis-muconic acid utilizing benzoate as a starting material (Journal of Biotechnology (2011) 156, 163-172).

Thus, a review of the literature illustrates that a significant need remains for improved, flexible, reliable, economical technologies that are capable of converting a wide variety of biomass to industrially relevant chemical intermediates and final products, especially technologies that are capable of converting all of the key constituents of biomass; e.g. lignin, cellulose, and hemicellulose. To achieve this goal, robust genetically modified microorganisms, and/or mixtures of microorganisms are required that are capable of funneling chemical compounds through multiple metabolic pathways to common a common precursor or precursors, that can be subsequently converted to useful chemical intermediates and final products. In addition, novel upstream and downstream processing techniques are needed to assist with biomass fractionation, lignin and polysaccharide depolymerization, and precursor conversion to chemical intermediates and final products. The concepts presented herein provide some technologies that address these and other needs.

### SUMMARY

An aspect of the present invention is a genetically modified microorganism that includes at least one exogenous gene addition, wherein the at least one added gene encodes at least one of a decarboxylase, a dehydratase, or a monooxygenase. In some embodiments of the present invention, a genetically modified microorganism may have at least one deleted gene that encodes at least one of a dioxygenase, a muconate lactonizing enzyme, or muconolactone isomerase. In some embodiments of the present invention, a microorganism may over-express at least one demethylase gene. In some embodiments of the present invention, a microorganism may include a deletion of at least one catabolite repression control gene.

In some embodiments of the present invention, the at least one exogenous gene may encode a decarboxylase from Enterobacter cloacae. In some embodiments of the present invention, the exogenous gene may be at least one of aroY, ecdB, or ecdD. In some embodiments of the present invention, the at least one exogenous gene may encode a dehydratase from Bacillus cereus or from P. pneumonia. In some further embodiments of the present invention, the exogenous gene may be at least one of aroZ or asbF. In some further embodiments of the present invention, the at least one exogenous gene may encode a monooxygenase from Pseudomonas putida CF600. In still further embodiments of the present invention, the exogenous gene may be at least one of dmpK, dmpL, dmpM, dmpN, dmpO, dmpP, or pheA. In still further embodiments of the present invention, the at least one deleted gene may be at least one of pcaH or pcaG. In some embodiments of the present invention, the at least one deleted gene from a microorganism may be at least one of catB or catC. In some embodiments of the present invention, the demethylase gene may be at least one of vanA, vanB, or ligM.

In some embodiments of the present invention, the microorganism may be at least one of a fungi, a prokaryote, or a prokaryotic microorganism. In some embodiments of the present invention, the microorganism may be a prokaryote or prokaryotic microorganism from the genus Pseudomonas. In some embodiments of the present invention, the microorganism may be a strain of P. putida, P. fluorescens, or P. stutzeri. In some further embodiments of the present invention, the microorganism may be a strain of P. putida KT2440.

A further aspect of the present invention is a process for producing muconic acid, where the process includes contacting a culture broth containing lignin depolymerization compounds with any of the genetically modified microorganism disclosed within this specification. In some embodiments of the present invention, the lignin depolymerization compounds may include at least one of p-coumaric acid, ferulic acid, benzoic acid, phenol, coniferyl alcohol, caffeic acid, vanillin, or 4-hydroxybenzoic acid, and at least a portion of the lignin depolymerization compounds are converted to catechol, and at least a portion of the catechol is converted to muconic acid.

A further aspect of the present invention is a process for producing adipic acid, where the process includes separating muconic acid from a culture broth comprising muconic acid, impurities, and microorganisms, purifying the separated muconic acid, and hydrogenating at least a portion of the purified muconic acid to produce the adipic acid or other chemicals. In some embodiments of the present invention, the separating may include at least one of centrifugation and/or filtration to produce muconic acid that is substantially free of the microorganism. In some embodiments of the present invention, the purifying may include contacting the separated muconic acid with an adsorbent, wherein the adsorbent removes at least a first portion of the impurities from the separated muconic acid.

In some embodiments of the present invention, the adsorbent may include activated carbon. In some embodiments of the present invention, the impurities removed may include at least one of benzoic acid, protocatechuic acid or 4-hydroxybenzoic acid. In some embodiments of the present invention, the purifying may include crystallizing at least a portion of the muconic acid from the separated muconic acid to form a muconic acid precipitate and a liquid that contains at least a portion of the impurities.

In some embodiments of the present invention, the purifying may include dissolving the muconic acid precipitate in a solvent, resulting in a liquid phase that includes muconic acid and a solid phase that includes at least a portion of the impurities, and separating the liquid phase from the solid phase. In some embodiments of the present invention, the separating may be by at least one of filtration or centrifugation. In some embodiments of the present invention, the hydrogenation may include contacting the liquid phase that includes muconic acid and diatomic hydrogen with a metallic catalyst. In some embodiments of the present invention, the metallic catalyst may include at least one of palladium, platinum, ruthenium, or rhodium. In some embodiments of the present invention, the at least one of palladium, platinum, ruthenium, or rhodium may be supported by activated carbon or silica. In some embodiments of the present invention, the metallic catalyst may include rhodium supported by activated carbon.

In an aspect, disclosed herein is a genetically modified microorganism comprising at least one exogenous gene addition, wherein the at least one added gene encodes at least one of a decarboxylase, a dehydratase, or a monooxygenase. In an embodiment, the genetically modified microorganism produces 33.7 g/L muconate at 0.18 g/L/h at a 46% molar yield. In an embodiment, the yield of the muconate produced is up to 92% of the maximum theoretical yield. In an embodiment, disclosed herein is a process for producing muconic acid, the process comprising contacting a culture broth containing lignin depolymerization compounds with the microorganism.

In an aspect, disclosed herein is a process for producing adipic acid, the process including separating muconic acid from a culture broth comprising muconic acid, impurities, and a microorganism; purifying the separated muconic acid; and hydrogenating at least a portion of the purified muconic acid to produce the adipic acid.

In addition to the exemplary aspects and embodiments described above, further aspects and embodiments will become apparent by reference to the drawings and by study of the following descriptions.

### BRIEF DESCRIPTION OF DRAWINGS

The accompanying drawings are incorporated into and form a part of the specification to illustrate examples of how the aspects, embodiments, or configurations can be made and used and are not to be construed as limiting the aspects, embodiments, or configurations to only the illustrated and described examples. Further features and advantages will become apparent from the following, more detailed description of the various aspects, embodiments, or configurations.

FIGS. 1a-c illustrate biorefinery processes for the conversion of biomass to chemical intermediates and final products, according to exemplary embodiments of the present invention.

FIG. 2 illustrates engineered metabolic pathways in P. putida for converting both lignin depolymerization products and polysaccharide depolymerization products to muconic acid, according to exemplary embodiments of the present invention.

FIG. 3 summarizes engineered modifications to the genome of a P. putida strain to increase the production of muconic acid, followed by conversion to chemical intermediates, according to exemplary embodiments of the present invention.

FIG. 4 summarizes experimental culture results obtained using an engineered strain of Pseudomonas putida metabolizing various substrates for their conversion to muconic acid, according to exemplary embodiments of the present invention.

FIG. 5 summarizes experimental results from a DO-stat fed-batch experiment utilizing an engineered strain of P. putida (KT2440-CJ103) to convert p-coumarate to muconic acid, according to exemplary embodiments of the present invention.

FIG. 6 summarizes experimental results from a DO-stat fed-batch experiment utilizing an engineered strain of P. putida (KT2440-CJ103) to convert benzoate to muconic acid, according to exemplary embodiments of the present invention.

FIGS. 7a-c summarize experimental results obtained from an engineered strain of P. putida that coexpresses decarboxylase subunits, EcdB and EcdD, to enhance the activity of the protocatechuate decarboxylase, AroY, according to exemplary embodiments of the present invention.

FIGS. 8a-d summarize experimental results obtained from an engineered strain of P. putida that over-expresses VanAB for enhanced conversion of vanillate to protocatechuate to enable enhanced muconic acid production, according to exemplary embodiments of the present invention.

FIGS. 9a-h summarize experimental results obtained from an engineered strain of P. putida with deregulation of Carbon Catabolite Repression to enhance aromatic catabolism and, subsequently, increase production of muconic acid from aromatic molecules, according to exemplary embodiments of the present invention.

FIGS. 10a-b summarize experimental results obtained from an engineered strain of P. putida modified to express (−)-3-dehydroshikimate dehydratase, AsbF, the protocatechuate decarboxylase, AroY, and the protocatechuate subunit, EcdB, for enhanced production of muconic acid from sugars, according to exemplary embodiments of the present invention.

FIG. 11 tabulates primer sequences used to genetically engineer some of the P. putida strains engineered for improved muconic acid production, according to exemplary embodiments of the present invention. Depicted are LP29 (SEQ ID NO:1), LP30 (SEQ ID NO:2), LP31 (SEQ ID NO:3), LP32 (SEQ ID NO:4), LP33 (SEQ ID NO:5), LP34 (SEQ ID NO:6), LP48 (SEQ ID NO:7), LP49 (SEQ ID NO:8), LP50 (SEQ ID NO:9), LP51 (SEQ ID NO:10), LP53 (SEQ ID NO:11), LP54 (SEQ ID NO: 12), oCJ100 (SEQ ID NO: 13), oCJ101 (SEQ ID NO:14), oCJ102 (SEQ ID NO: 15), oCJ103 (SEQ ID NO:16), oCJ165 (SEQ ID NO:17), and oCJ166 (SEQ ID NO:18).

FIGS. 12a-c show experimental results comparing muconic acid production of a genetically engineered strain to the starting strain, according to exemplary embodiments of the present invention.

FIG. 13 illustrates post-biocatalysis steps to produce purified muconic acid and subsequently final produces, according to exemplary embodiments of the present invention.

FIG. 14 illustrates experimental results quantifying the components present in a culture broth before treatment with activated carbon, after treatment with activated carbon, and after crystallization, according to exemplary embodiments of the present invention.

FIG. 15 compares the elemental content of commercially available muconic acid biocatalysis-derived muconic acid at various purification/separation steps, according to exemplary embodiments of the present invention.

FIGS. 16a-f illustrate purification of muconic acid biological culture media by activated carbon treatment, pH/temperature shift crystallization, and ethanol dissolution with microfiltration, according to exemplary embodiments of the present invention.

FIGS. 17a-d summarize data from experiments evaluation catalytic hydrogenation of muconic acid to adipic acid, according to exemplary embodiments of the present invention.

FIG. 18 illustrates experimental data obtained screening catalysts for hydrogenating muconic acid to adipic acid using noble metal catalysts, according to exemplary embodiments of the present invention.

FIGS. 19a-j summarize XRD spectra of virgin catalysts used for batch reactor screening experiments and after metal loading and catalyst reduction, according to exemplary embodiments of the present invention. Spectra were also provided for blank powdered silica and activated carbon supports for reference (a, b).

FIGS. 20a-b illustrate muconic acid hydrogenation activity for platinum group metals (PGM) on powder activated carbon and silica supports in batch (a), and PGM leaching after 35 min exposure to reaction conditions (b), according to exemplary embodiments of the present invention.

FIGS. 21a-j summarize data from batch reactor catalyst activity screening for muconic acid hydrogenation), according to exemplary embodiments of the present invention. Reactions were performed in a minimum of duplicate batch reactors, with error bars indicating standard deviations. Reaction conditions were as follows: temperature 24° C., muconic acid 200 mg, catalyst loading 15 mg, hydrogen pressure 24 bar, ethanol solvent 19.8 g.

FIG. 22 illustrates product molar yields, catalyst bed temperature, and liquid feed rate during the 100-h time-on-stream stability test of 1% Rh/AC for muconic acid hydrogenation, according to exemplary embodiments of the present invention. Reaction conditions were as follows: muconic acid 1 wt % in ethanol, temperature and liquid flow rate as indicated, hydrogen 200 seem at 24 bar, catalyst loading 1100 mg.

FIG. 23 illustrates preliminary trickle bed reactor results with 1% Pd/AC granules, demonstrating ˜12 h to reach stead-state activity, according to exemplary embodiments of the present invention. Reaction conditions were as follows: muconic acid 1 wt % in ethanol, liquid flow rate 0.5 mL/min, catalyst bed temperature 72° C., hydrogen 200 seem at 24 bar, and catalyst loading 200 mg of 1% Pd/AC granules.

FIG. 24 illustrates a polymerization scheme for reacting bio-adipic acid with 1,6-hexanediamine to produce Nylon-6,6 (left), according to exemplary embodiments of the present invention. This method using bio-adipic acid produced from the catalytic hydrogenation of muconic acid (right). Adipic acid was initially reacted with thionyl chloride and dissolved to cyclohexane, prior to adding the solution to a basic 1,6-hexanediamine aqueous solution. Nylon “rope” was then pulled from the biphasic solution interface for subsequent characterization.

FIG. 25 illustrates a flow diagram for the separation/purification and upgrading areas of a biorefinery for the conversion of lignocellulosic materials to muconic acid and subsequently to adipic acid, according to exemplary embodiments of the present invention.

FIG. 26 depicts Muconate production from glucose and xylose. a Schematic of the overall metabolic engineering strategy. To utilize xylose, xylE, D-xylose isomerase (xylA), xylulokinase (xylB), transaldolase (tal) and transketolase (tkt) from E. coli were heterologously expressed. E4P and PEP were condensed to form DAHP via a feedback-resistant DAHP synthase (aroG^{D146N}). To convert DAHP to muconate (MA), genes encoding a 3-DHS dehydratase (asbF) from Bacillus cereus, and a PCA decarboxylase (aroY) and its corresponding co-factor generating protein (ecdB), both from Enterobacter cloacae, were heterologously expressed. aroB and catechol 1,2-dioxygenase (catA) were overexpressed.^{3,32} An engineered chorismate pyruvate-lyase from E. coli (ubiC-C22)⁴⁰ was overexpressed to convert chorismate (CSA) to 4-hydroxybenzoate (4HB), which can be converted to PCA and MA. Deleted genes are shown in red. Glucose dehydrogenase (gcd) was deleted to prevent formation of xylonate or gluconate. Glucose-6-phosphate isomerases pgi-1 and pgi-2 were each deleted previously,^{3,32} but pgi-1 was restored in this study. Pyruvate kinases pykA and pykF were each deleted to reduce competition for PEP. To accumulate MA, pcaHG and catBC were deleted to prevent ring-opening of PCA and catabolism of MA, respectively. Abbreviations not described above: P: phosphate; 2-KGn: 2-ketogluconate; 2-KG-6-P: 2-ketogluconate-6-P; G6P: glucose-6-P; 6PG: 6-phosphogluconate; KDPG: 2-keto-3-deoxy-6-phosphogluconate; G3P: glyceraldehyde-3-P; FBP: fructose-1,6-P₂; F6P: fructose-6-P; S7P: sedoheptulose-7-P; R5P: ribose-5-P; Ri5P: ribulose-5-P; 3PG: 3-phosphoglycerate; CAT: catechol; SA: shikimate; S3P: shikimate-3-phosphate; ICIT: isocitrate; CIT: citrate; AKG: alpha-ketoglutarate; SUCC: succinate; FUM: fumarate; MAL: malate; GLX: glyoxylate; OAA: oxaloacetate; AcCoA: acetyl-Coenzyme A. b Metabolic modelling of the maximum theoretical muconate molar and carbon yields with or without pgi-1. The blue lines represent the asbF pathway, the red lines represent the aroE pathway, solid lines represent molar % yield, dash lines represent carbon % yield. The grey areas represent the molar percentage of xylose consumed from 33% to 40% (with the balance glucose), mimicking the composition of corn stover hydrolysates. c Shake-flask cultivations of strain QP328 on glucose and xylose. % Molar yield was calculated as [mM muconate/mM (glucose+xylose)×100], % carbon yield was calculated as [mM muconate ×6/mM (glucose ×6+xylose ×5)×100]. Error bars represent the standard deviation of biological triplicates.

FIG. 27 depicts an evaluation of reverse engineered strains comparing to QP478. a Mutations identified in ALE by whole genome sequencing of QP478. b Growth curve of reverse engineered strains LC091 and LC100, with the unevolved strain QP328 and the evolved strain QP478 for comparison. μ_{A} represents absolute growth rate, all μ_{A} presented here are the average values of at least 3 independent growth curves. c Shake flask experiments of reverse engineered strains LC091 and LC100, comparing to QP478, on M9 medium supplemented with ˜30 mM xylose. Yield of LC100 was compared to LC091 using two-tailed student t-test (p<0.0001). d Shake flasks experiments comparing reverse engineered strains LC091, LC100 and evolved strain QP478 on M9 medium supplemented with ˜30 mM glucose and ˜15 mM xylose. % Molar yield was calculated as [mM muconate/mM (glucose+xylose)×100], % carbon yield was calculated as [mM muconate ×6/mM (glucose ×6+xylose ×5)×100]. Error bars here represent the standard deviation of three biological replicates.

FIG. 28 depicts characterization of rationally engineered strains overexpressing genes from the duplicated region. a Identification of the duplicated region from next generation sequencing data, which presented as ˜2× the number of sequencing reads in QP478, and complete and partial deletions of this duplicated regions in LC171 and LC173, respectively. The graphs of coverage were generated in Geneious Prime 2020.0.4. b Growth curves of QP478, LC171, and LC173 on M9 medium with ˜30 mM xylose. λ represents growth lag, μ_{A} represents absolute growth rate, both are the average values of at least 3 independent growth curves. c Overexpressing candidate genes in reverse engineered strain LC100 at the ΔpykF site. d Growth curves of QP478, LC100, LC199 and LC224 on M9 medium with ˜30 mM xylose. e Maximum specific growth rates extracted from panel c. f Growth lag values extracted from panel c. g-i Profiles in shake flask experiments of strain LC224 on M9 medium with ˜30 mM xylose,˜30 mM glucose+˜15 mM xylose, ˜30 mM glucose, respectively. For shake flask experiments, % molar yield was calculated as [mM muconate/mM (glucose+xylose)×100], % carbon yield was calculated as [mM muconate ×6/mM (glucose ×6+xylose ×5)×100]. Error bars here represent the standard deviation of three biological replicates.

FIG. 29 depicts bioreactor profiles from strains QP478 and LC224 in fed-batch mode. a, b Bacterial growth, glucose and xylose utilization, and muconate titers, yields, and rates from QP478 and LC224 in 96.6-hour cultivations. c Bacterial growth, glucose and xylose utilization, and muconate titers, yield, and rate from LC224 in 191-hour cultivation. For a and b, data points represent the average values of biological duplicates, error bars represent the absolute difference between the data generated from duplicates at each time point; for c, data points represent the values of singlets. Metabolic yields (mol %) at each time point were calculated as the amount of muconate (in moles) produced divided by the glucose and xylose (in moles) utilized. Metabolic yields (mol %) are corrected based on the dilution factor generated by the volumes of base and feeding. Final carbon yields (carbon %) listed were calculated as [mM muconate ×6/mM (glucose ×6+xylose ×5)×100]. Rates (g/L/h) at each time point were calculated as the muconate concentration divided by the cultivation time. All the titer (T), rate (R), and yield (Y) listed were values at the last time point. Final yields (mol %, carbon %) listed in c has been also corrected based on the quantified evaporated volume.

FIG. 30 depicts selected intracellular and extracellular metabolomics analysis of strains grown on glucose and xylose. a Simplified metabolic pathway. Metabolites in the EDEMP cycle were labeled blue, in the shikimate and muconate pathway were labeled green, the joint node DAHP was labelled purple, the extracellular anthranilic acid (ANA) was labeled brown. Abbreviations in the figure not described above: QA: quinic acid; ANA: anthranilic acid. b Intensity of selected metabolites in the three strains QP328 (blue), QP478 (orange), and LC224 (red). Intracellular intensities have been normalized by lyophilized biomass. Error bars represent standard deviation of three biological replicates.

### REFERENCE NUMBERS

100 biorefinery
110 lignocellulosic biomass
120 pretreatment-fractionation
130 lignin depolymerization
135 polysaccharide depolymerization
140 microbial catalysis
150 separation/purification
160 catalytic hydrogenation
170 upgrading
200 bioreactor
210 muconic acid containing culture broth
220 purified muconic acid
230 final products

### DETAILED DESCRIPTION OF EXEMPLARY EMBODIMENTS

The following detailed description illustrates the invention by way of example and not by way of limitation.

Muconic acid is a bioprivileged molecule that can be converted into direct replacement chemicals for incumbent petrochemicals and performance-advantaged bioproducts. In this study, Pseudomonas putida KT2440 was engineered to convert glucose and xylose, the primary carbohydrates in lignocellulosic hydrolysates, to muconic acid using a model-guided strategy to maximize the theoretical yield. Using adaptive laboratory evolution (ALE) and metabolic engineering in a strain engineered to express the D-xylose isomerase pathway, we demonstrated that mutations in the heterologous D-xylose:H+ symporter (XylE), increased expression of a major facilitator superfamily transporter (PP_2569), and overexpression of aroB encoding the native 3-dehydroquinate synthase, enable efficient muconic acid production from glucose and xylose simultaneously. Using the rationally engineered strain, we produced 33.7 g/L muconate at 0.18 g/L/h and a 46% molar yield (92% of the maximum theoretical yield). This engineering strategy is exceptionally promising for the production of other shikimate pathway-derived compounds from lignocellulosic sugars. This disclosure also relates to compositions and methods for converting biomass to various chemical intermediates and final products including fuels. Aspects include the depolymerization of lignin, cellulose, and hemicellulose to a wide slate of depolymerization compounds that can be subsequently metabolized by genetically modified bacterium, and converted to cis,cis-muconic acid. Other aspects include the use of monometallic catalysts for converting the cis,cis-muconic acid to commodity chemicals and fuels, for example adipic acid and/or nylon.

Several previous efforts to produce muconate from sugars via asbF have disrupted the shikimate pathway by deleting aroE. Deletion of aroE results in strains that are auxotrophic for essential aromatic amino acids, which is undesirable for a bioprocess. Recently, Pseudomonas putida KT2440 (hereafter P. putida) strains have been engineered to efficiently produce muconate from glucose via asbF. Most recently, we reported engineering of P. putida that achieved a titer of 22.0 g/L at 0.21 g/L/h and a 35.6% molar yield from glucose in a pH-controlled bioreactor.

To date, most efforts to produce muconate from carbohydrates have employed glucose as a substrate. However, the co-utilization of glucose and xylose—often are the two major carbohydrates in lignocellulose—is crucial for the valorization of sugar hydrolysates. Co-utilization of glucose and xylose for muconate production has been studied in Escherichia coli. In this previous work, xylose was metabolized to the TCA cycle to avoid the carbon catabolite repression (CCR), thus limiting muconate yield, which motivates development of other strategies towards this goal. Unlike E. coli, P. putida is natively unable to utilize xylose, which provides an opportunity to engineer optimal xylose pathways in the absence of CCR.

In the current study, we sought to incorporate xylose utilization to achieve efficient muconate production from glucose and xylose in P. putida. To this end, we first deleted hexR and engineered the D-xylose isomerase pathway into a strain previously engineered to produce muconate from glucose (Table 7). By combining metabolic modeling, rational strain engineering, adaptive laboratory evolution, and bioreactors cultivation, we identified successful strategies to improve muconate production from glucose and xylose. Finally, metabolomics was performed to infer the impact of the genetic modifications on metabolic flux.

TABLE 7
An embodiment of strains disclosed herein.

| Strain | Genotype | References |
|---|---|---|
| CJ522 | P. putida KT2440 | Johnson et al., |
| | ΔcatRBC::Ptac:catA | 2019 |
| | ΔpcaHG::Ptac:aroY:ecdB:asbF | |
| | ΔpykA::aroG-D146N:aroY:ecdB:asbF | |
| | ΔpykF Δppc Δpgi-1 Δpgi-2 Δgcd | |
| JE3226 | P. putida KT2440 | Elmore et al., |
| | ΔhsdR::P_{tac}:BxB1int-attB Δgcd | 2020 |
| | ΔampC::P_{xylE*}xylE:xylAB:tktA:talB | |
| LC041 | JE3226 Δpgi-1 | This study |
| LC345 | JE3226 Δpgi-2 | This study |
| LC347 | LC041 Δpgi-2 | This study |
| QP328 | CJ522 ΔhexR | This study |
| | ΔampC::P_{xylE*}xylE:Ptac:xylAB:talB:tktA | |
| | Δpgi-1::pgi-1 PP_1736- | |
| | 1737(intergenic)::Plac:ubiC-C22 | |
| QP478 | QP328 xylE-A62V, A455V P_{PP}_{_{—}}₂₅₆₉ | This study |
| | G→A duplication of PP_5050- | |
| | PP_5242 | |
| LC061 | QP478 restoration G→A at | This study |
| | P_{PP}_{_{—}}₂₅₆₉ | |
| LC075 | QP478 restoration of xylE-A62V, | This study |
| | A455V | |
| LC078 | QP478 restoration of xylE-A455V | This study |
| LC093 | QP478 restoration of xylE-A62V | This study |
| LC091 | QP328 xylE-A62V, A455V | This study |
| LC092 | QP328 P_{PP}_{_{—}}₂₅₆₉ G→A | This study |
| LC100 | LC091 P_{PP}_{_{—}}₂₅₆₉ G→A | This study |
| LC147 | LC100 ΔpykF::P_{lac}:gpmI | This study |
| LC150 | LC100 ΔpykF::P_{tac}:maeB | This study |
| LC151 | LC100 ΔpykF::P_{tac}:rpiA | This study |
| LC168 | LC100 ΔpykF::P_{tac}:aroK:aroB | This study |
| LC171 | QP478 ΔPP_5050-PP_5242 | This study |
| LC173 | QP478 ΔPP_5084-PP_5242 | This study |
| LC199 | LC100 ΔpykF::P_{tac}:aroK | This study |
| LC224 | LC100 ΔpykF::P_{tac}:aroB | This study |
| LC349 | QP328 ΔpykF::P_{tac}:aroB | This study |

*Represents mutation in promoter of xylE.

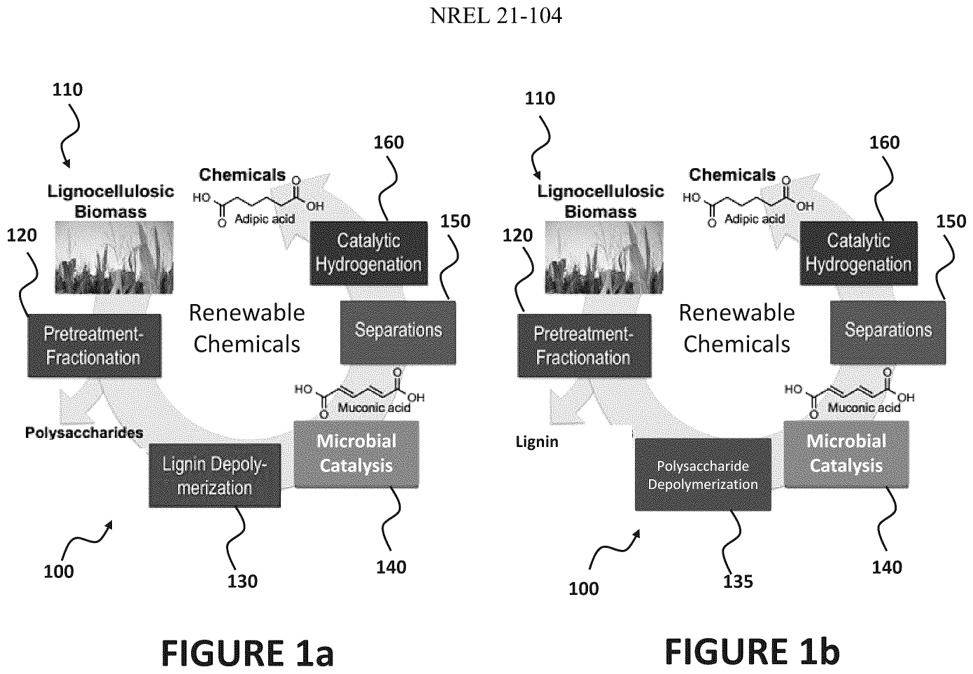

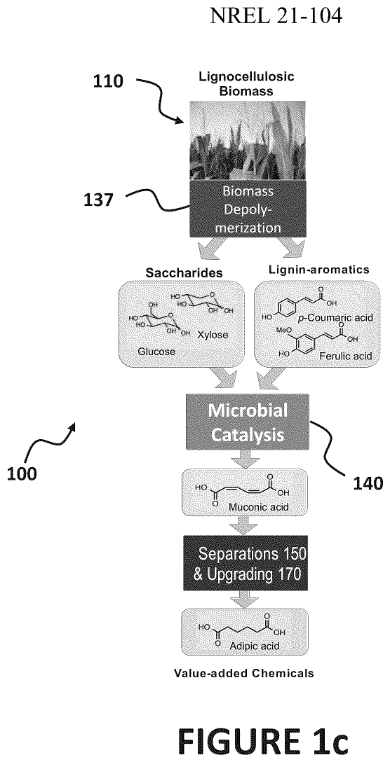

Introducing the D-xylose isomerase pathway into muconate-producing P. putida. Three xylose metabolic pathways were considered to enable production of muconate from this substrate, including the isomerase pathway in which xylose is metabolized to xylulose-5-P (X5P) in the pentose phosphate pathway (PPP), the Weimberg pathway that feeds xylose to the TCA cycle via α-ketoglutarate, and the Dahms pathway, which shares the initial three steps with the Weimberg pathway, after which α-ketoglutaric semialdehyde is converted by an aldolase into pyruvate and glycolaldehyde. Among these, the D-xylose isomerase pathway, in which xylose is metabolized via the D-xylose isomerase (xylA) and xylulokinase (xylB) to xylulose-5-phosphate (X5P), is ideal for achieving a high theoretical muconate yield since X5P can be further converted to E4P and subsequently enter the shikimate pathway (FIG. 1a). We integrated the isomerase pathway into a strain previously engineered to produce muconate from glucose, CJ5223, by overexpressing codon-optimized versions of the E. coli D-xylose isomerase (xylA), xylulokinase (xylB) and D-xylose:H+symporter (xylE), together with a transaldolase (tal) and a transketolase (tkt) to improve carbon flux within the PPP (FIG. x1a). We also deleted hexR, which encodes a transcriptional regulator that controls expression of genes important for sugar metabolism, since we had previously found this to improve conversion of glucose to muconate.

Thompson et al. previously reported that employing both the asbF and aroE pathways can help to maximize net precursor assimilation and metabolite flux toward muconate. Thus, an engineered chorismate pyruvate-lyase (ubiC-C22)41 with relieved product inhibition was integrated to enhance muconate production through the shikimate pathway via aroE (FIG. 1a). We had previously deleted pgi-1 and pgi-2, which encode redundant glucose-6-P isomerases, to disrupt the EDEMP cycle, a combination of the Entner-Doudoroff, gluconeogenic Embden-Meyerhoff-Pernass, and the pentose phosphate pathways. The purpose of disrupting the EDEMP cycle is to prevent it from cycling to generate pyruvate independent of PEP during growth on glucose, which could enable the cell to redirect carbon toward growth at the expense of muconate production despite deletion of the genes encoding the pyruvate kinases (pykA, pykF) and PEP carboxylase (ppc). This strategy is beneficial for muconate production from glucose as the sole carbon source, but in this case, deletion of pgi-1 and pgi-2 would decrease the maximum theoretical muconate yield of both asbF- and aroE-catalyzed muconate biosynthesis pathways when xylose is converted via the PPP (FIG. 1b).

Considering that the xylose fraction in the mixture of glucose and xylose (xylose/glucose+xylose %, moles) in corn stover hydrolysate ranges from 34.3% to 38.4%, the modeling predicted maximum theoretical yield of muconate with pgi-1 and pgi-2 deleted to be lower than if one or both are present (FIG. 1b). To prove the concept that glucose-6-phosphate isomerase (encoded by pgi-1 and pgi-2) activity is necessary for xylose flux entering the EDEMP cycle, we built relevant strains based on the wild-type derivative strain JE3226 which was previously engineered to utilize xylose using the D-xylose isomerase pathway, generating strains LC041 (Δpgi-1), LC345 (Δpgi-2), LC347 (Δpgi-1 Δpgi-2). In the plate reader evaluation on M9 medium containing xylose, LC347 failed to grow, both LC041 and LC345 demonstrated decreased growth rates and increased growth lags. Specifically, LC345 with pgi-1 intact exhibited lower growth rate and longer growth lag compared to LC041, suggesting that Pgi-1 has lower activity relative to Pgi-2. Considering that EDEMP cycle would compete with muconate biosynthesis and may lead to lower muconate yield, we thus restored pgi-1, the one exhibited lower activity, to enable xylose flux into the EDEMP cycle and improve the maximum theoretical yield, generating strain QP328 (FIG. 1a, Table 1).

Strain QP328 was cultivated in shake flasks on a mixture of glucose and xylose to examine their conversion to muconate. Although the xylose isomerase pathway has been shown to be efficient in wild-type P. putida,35 the xylose utilization rate of QP328, however, was very low compared to that of glucose (FIG. 1c). Since glucose and xylose can be utilized simultaneously in the P. putida KT2440 wild-type background upon introduction of the same xylose isomerase pathway,35 we hypothesized that a bottleneck in xylose transport or metabolism was present in our muconate-producing strain.

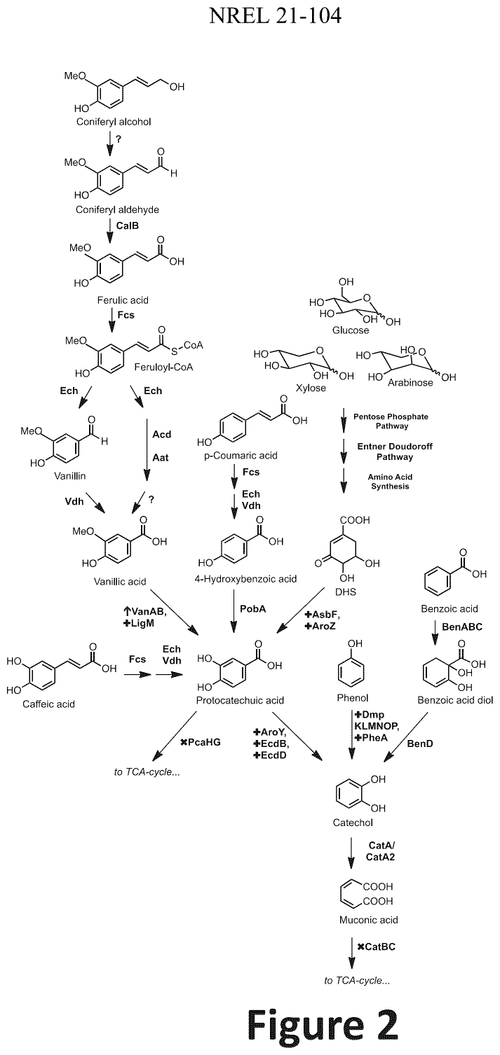

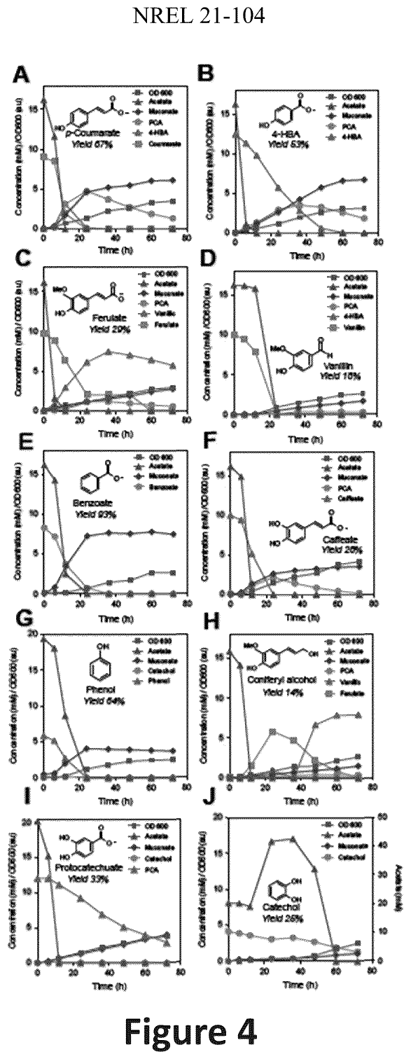

ALE of QP328 to improve growth on xylose. To improve xylose utilization by QP328, we conducted ALE by serial passaging of the strain on M9 medium supplemented with 10 mM xylose as a sole carbon and energy source. As the populations were passaged, higher OD600 values were achieved more rapidly. After 7 passages (approximately 50 generations), all 4 lineages achieved turbidity in 2-4 days compared to 14 days at the beginning of the ALE, and the evolution was terminated. The evolved populations of the 4 lineages were plated onto an LB agar plate and 3 isolates on each plate were chosen for shake flasks pre-screening (12 isolates in total). In most cases, all triplicates from the same lineage exhibited similar growth and muconate production, so it was assumed that they likely represented the same genotype and only one from each lineage was saved. In lineage 1, however, one replicate performed differently, thus two isolates were saved. To identify mutations that may contribute to improved xylose utilization, the genomes of all five isolates were sequenced. Four of the isolates (1, 3-5) had mutations that likely inactivated aroG-D146N (frame shift+7 bp, frame shift+2 bp, M1N and L2H, frame shift −16 bp, for isolate 1, 3, 4, 5, respectively). The five isolates were then evaluated in shake flasks on glucose, xylose, and a mixture of glucose and xylose. As expected, strains with mutations in aroG-D146N grew better but produced less muconate. Isolate 2 achieved the highest muconate yield and the lowest biomass yield and was designated QP478 (Supplementary FIG. 4). QP478 demonstrated substantially improved growth on xylose compared to QP328 in a plate reader, in which the growth of QP328 was negligible while QP478 reached a OD600 of 0.5 in 72 hours (FIG. 2b).

The mutations identified in QP478 are hypothesized might be related to the improved growth on xylose included: 1) two missense mutations in the xylose transporter gene, xylE, where alanine residues were replaced with valines, A62V and A455V; 2) a G to A point mutation 10 bp upstream of the ˜35 element of a putative promoter predicted by the BPROM σ70 promoter prediction software44 upstream of PP_2569, which is annotated as a metabolite major facilitator superfamily (MFS) transporter in the Uniprot database; and 3) a 227.8 kB region of the genome from PP_5050 to PP_5242 that appeared to be duplicated (FIG. 2a).

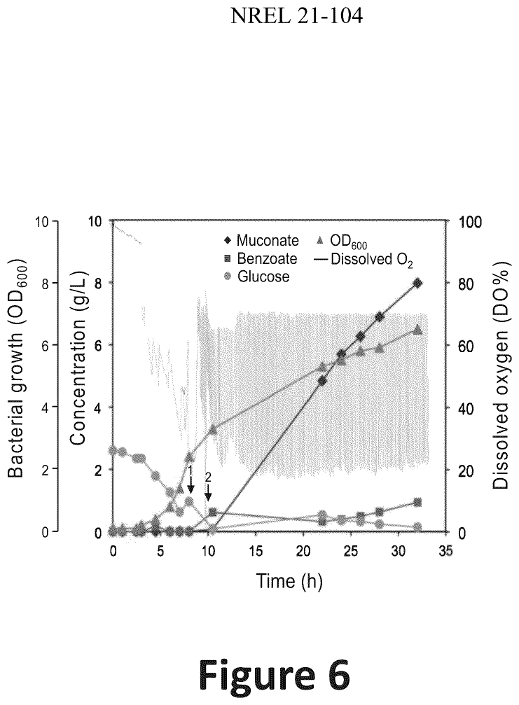

Evaluation and reverse engineering of the ALE-derived mutations. To understand the contribution of the mutations that led to improved growth on xylose during ALE, we created strains that individually restored the wild-type sequences into the evolved strain QP478. The A62V and A455V mutations were restored to wild type separately in xylE were restored separately, generating LC093 and LC078, respectively. The G Q A mutation in the promoter region of PP_2569 was restored, generating LC061. In the plate reader evaluation, restoring either xylE-A455V or xylE-A62V led to decreased growth rate and increased growth lag of LC078 and LC093. The restoration of the G to A mutation in PPP_2569 led to slightly decreased growth rate (Supplementary FIG. 6a, c). In shake flasks experiment, only LC093 demonstrated significantly lower muconate yield compared to QP478, all the three strains LC061, LC078 and LC093 exhibited slower growth and muconate production, which is consistent with the results in plate reader. Reduced performances of these strains, mainly slower muconate production caused by decreased growth rates and/or increased growth lag, indicated that all these mutations contributed to improved cell growth on xylose of QP478.

We also performed the reverse experiment, engineering the ALE mutations into the parent strain QP328 to obtain a rationally engineered strain containing only mutations that contribute to improved production of muconate. We first reverse engineered the unevolved strain QP328 with the three point mutations. The A62V and A455V XylE mutations were introduced into the unevolved strain QP328, generating LC091. The G to A mutation in PPP_2569 was engineered in QP328 and LC091, generating LC092 and LC100, respectively. Strains LC091, LC092, and LC100, together with QP328 and QP478, were evaluated in a plate reader containing M9 medium with 30 mM xylose. Interestingly, introducing the two XylE mutations enabled cell growth on xylose in LC091 (FIG. 2b), which exhibited a comparable growth rate but higher final biomass and lower muconate yield compared to QP478 on xylose alone and xylose and glucose mixtures (FIG. 2b). Introducing the G to A mutation in PPP_2569 to QP328 also enabled cell growth of LC092 on xylose, while at a much lower rate compared to LC091 (FIG. 2b). Unexpectedly, introducing the G to A mutation in PPP_2569 to LC091 led to decreased growth and lower biomass of LC100 on both xylose and mixed substrates (FIG. 2xb).

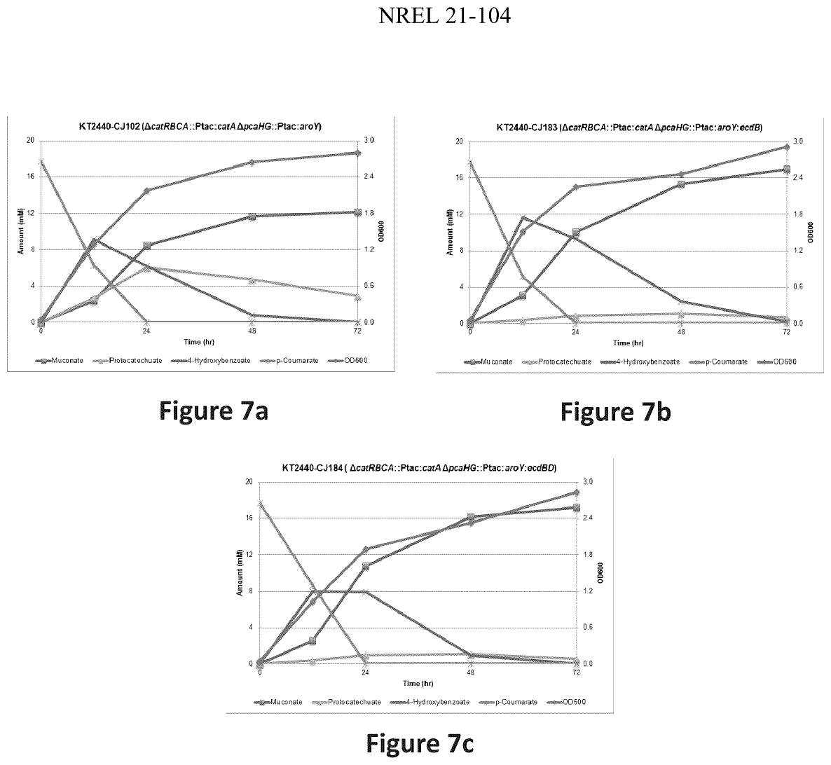

We next evaluated LC091, LC100, and QP478 in shake flasks containing M9 medium with 30 mM xylose. LC091 reached almost twice the biomass yield (OD600) but achieved lower muconate yield compared to QP478 (FIG. 2c, Supplementary Table 1). Moreover, LC100 reached a comparable muconate yield to QP478, albeit at a lower rate (FIG. 2c). Interestingly, the only difference between strains LC091 and LC100 is the G to A mutation in PPP_2569, RT-qPCR indicated that the G to A mutation in PPP_2569 increased the expression of PP_2569 (Supplementary FIG. 7). The function of PP_2569 remains unknown.

We also evaluated LC100 and QP478 on M9 medium with a mixture of glucose and xylose. With this mixture, the growth rate of LC100 was still much lower than that of QP478, though it utilized glucose and xylose simultaneously (FIG. 2d, Supplementary Table 1). The difference in these strains suggested that a gene or genes in the PP_5050-PP_5242 duplicated region might be important for the improved performance of QP478.

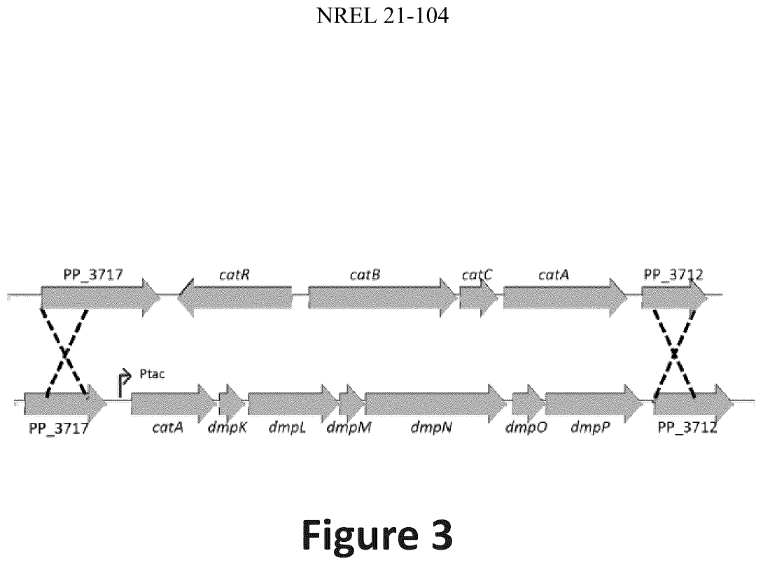

Investigation of the PP_5050-PP_5242 duplication. The 227.8 kB duplication was identified based on approximately 2-fold higher sequencing coverage from PP_5050 to PP_5242 compared to the rest of the genome (FIG. 3a). However, it was challenging to identify the exact location of the duplicated region based on the sequencing data due to the short read length of Illumina sequencing.45, 46 We thus deleted the original region of PP_5050-PP_5242 in QP478, using the known sequences outside the region as homologous arms, to generate LC171, and deleted a portion of the duplication from PP_5084-PP_5242 to generate strain LC173. On 30 mM xylose in M9 medium, LC171 with the whole region deletion exhibited a lower growth rate, while LC173 with partial deletion showed slightly improved growth with a reduced lagging time relative to QP478 (FIG. 3b). Based on these results, we concluded that the duplication is important to the performance of QP478, and the potential beneficial gene(s) should be in the region PP_5050-PP_5083, which remains intact in LC173. Thus, we next sought to identify the gene(s) in this region that contributed to improved growth on xylose.

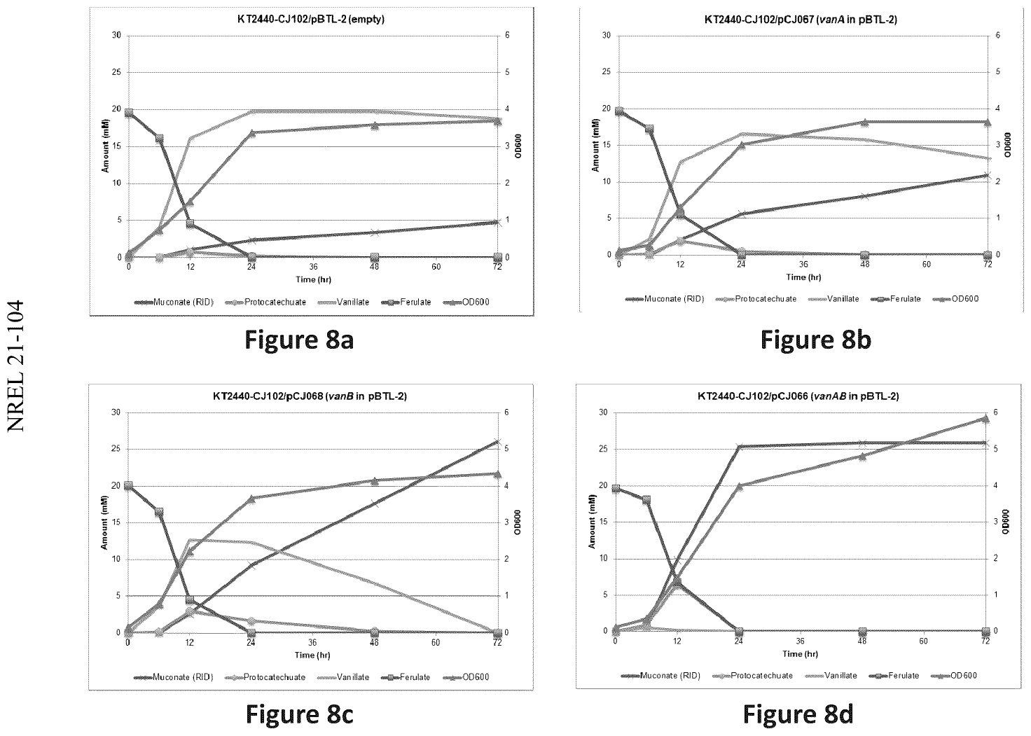

Since glucose and xylose were both utilized at similarly low rates in LC100 (FIG. 2d), we reasoned that the slow growth might manifest in part(s) of the pathway shared by both sugars. Three candidate genes within PP_5050-PP_5083 were selected for overexpression in LC100, including one related with sugar metabolism, PP_5056 (gpmI, 2,3-bisphosphoglycerate-independent phosphoglycerate mutase), and two in the shikimate pathway, PP_5078 (aroB, 3-dehydroquinate synthase) and PP_5079 (aroK, shikimate kinase) (FIG. 3c). Two other genes outside this region but related with sugar metabolism were also tested, including PP_5085 (maeB, malic enzyme B) and PP_5150 (rpiA, ribose-5-phosphate isomerase A. Overexpression cassettes of the five genes were then inserted individually at the ΔpykF site, generating strains LC147 (gpmI), LC150 (maeB), LC151 (rpiA), LC199 (aroK), and LC224 (aroB). All of these genes were driven by the Ptac promoter except for gpmI, which was driven by Plac promoter after two unsuccessful attempts to insert the gene into the genome using Ptac. The resulting strains were then evaluated with LC100 and QP478 in a plate reader containing M9 medium and 30 mM xylose (FIG. 3d-f). Overexpression of the three genes related to sugar metabolism in LC100 led to reduced cell growth (Supplementary FIG. 8). However, LC199 and LC224, which overexpress aroK and aroB, respectively, both demonstrated improved growth rates compared to LC100 (FIG. 3d). LC224 grew even faster than QP478 with a reduced lagging time and higher growth rate (FIG. 3d-f).

Strains LC199, LC224, LC168, and QP478 were evaluated in shake flask experiments with M9 medium containing glucose and xylose to examine muconate production. The aroB overexpression strain LC224 outperformed its evolved counterpart QP478 with a higher muconate yield and improved growth rate, suggesting that the reaction of DAHP to 3-dehydroquinate (3DHQ) was rate limiting in LC100. Overexpressing aroK in LC100 (generating LC199) increased the growth rate slightly. To investigate the potential additive effect of overexpressing aroK and aroB, we also expressed aroK and aroB in an operon-like pattern as aroKB in LC100, generating strain LC168, which did not exhibit improvement compared to LC224.

To investigate if aroB overexpression alone can lead to better strain performance, we overexpressed aroB in QP328, generating strain LC349. In the plate reader evaluation of strains LC349, QP328 and LC224, LC349 exhibited highest growth rate on glucose, and slightly lower growth rate on mixture of glucose and xylose compared to LC224, while not surprisingly much slower growth on xylose relative to LC224, probably due to the lack of mutations in xylE. Interestingly, in shake flasks experiment on mixture of glucose and xylose, LC349 outperformed QP328 with a much higher muconate yield, which was still significantly lower than LC224 (Supplementary FIG. 1id-f). The muconate production of LC349 is slower than LC224, as it took up to 41 hours for LC349 and 26.5 hours for LC224 to reach maximum muconate titer.

We next examined the performance of LC224 on M9 medium containing various substrates, including glucose, xylose, and mixture of glucose and xylose. The muconate yields were highest on xylose, while lowest on glucose (FIG. 3g-i), indicating the importance of introducing xylose into pentose phosphate pathway to supply E4P. Interestingly, both glucose and xylose utilization rates were higher on the mixture of glucose or xylose compared to glucose and xylose alone, respectively. This may suggest an addictive effect of glucose and xylose metabolism in our pathway design.

Bioreactor cultivations to assess strain performance. Bioreactor cultivations of LC224 and QP478 were conducted in fed-batch mode to maintain sugar (glucose and xylose) concentrations lower than 10 g/L. Glucose and xylose were simultaneously utilized in both strains from the start of the cultivation (FIG. 4a, 4b); however, sugar utilization rates were higher in LC224 than QP478. LC224 utilized 46 g/L glucose and 20 g/L of xylose by the end of the cultivation while QP478 utilized 34 g/L and 10 g/L, respectively. The muconate titer was almost 3-fold higher in LC224 compared to QP478, a 26.8 g/L and 9.3 g/L, respectively (FIG. 4a, b, Supplementary Table 1). Muconate yields reached 50% (molar yield) in LC224 while the yields were 25.9% in QP478 (FIG. 4a, 4b). These improvements in LC224 were also reflected in the overall muconate production rate (0.28 g/L/h), which was substantially higher than that achieved in QP478 (0.10 g/L/h) (FIG. 4a, b).

The muconate titer, rate, and yield achieved in bioreactor cultivations were 26.8 g/L, 0.28 g/L/h, and 49.9% (FIG. 4b), respectively, at 96.6 hours. This yield represents almost 100% of the maximum theoretical based on our strain design and metabolic modeling (vida supra). To explore whether the titer could be further improved, we conducted another bioreactor experiment where LC224 was cultivated for 191 hours (FIG. 4c). The resulting muconate titer increased to 33.7 g/L and at a yield of 46%, 92% of the theoretical maximum when corrected for evaporation. It is also worth noting that while LC224 reached stationary phase at ˜54 h, the cells continued utilizing sugars and producing muconate, which demonstrated that the muconate production here was not growth coupled, the muconate titer and yield could be further improved if the experiment had continued (FIG. 4b, c).

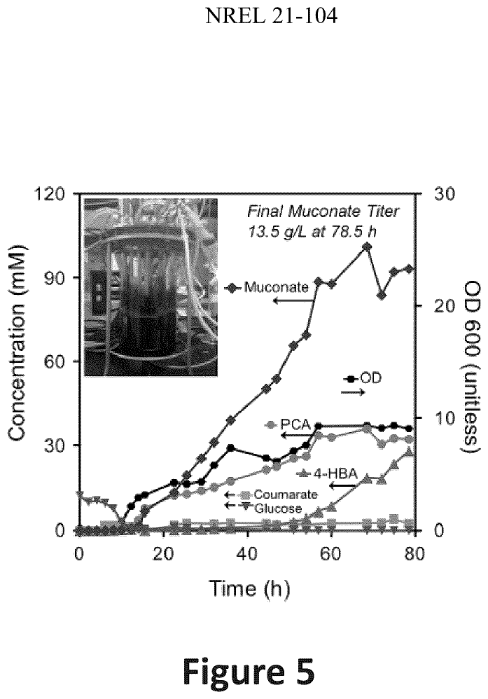

Metabolomic analysis of QP328, QP478, and LC224 cultivated on glucose and xylose. To better understand the differences between the unevolved parent QP328, the evolved strain QP478, and the rationally engineered strain LC224, intracellular and extracellular metabolomics experiments were conducted. Selected metabolites related to sugar metabolism and muconate production are presented in FIG. 5, with all quantified metabolites included in Supplementary Table 4. Compared to QP328, the strains QP478 and LC224 exhibited reduced accumulation of metabolites in the EDEMP cycle, and greater accumulation of metabolites in the shikimate and the muconate pathways (FIG. 5a, b), which is consistent with the mutations that evolved in QP478 and were engineered in LC224 (FIG. 2, 3). The intensities of DAHP, the joint node of sugar metabolism and shikimate pathway, however, demonstrated opposite pattern compared to other metabolites in shikimate pathway (FIG. 5b). LC224 and QP478 accumulated less DAHP compared to QP328, which is consistent to our objective above regarding the duplication and aroB overexpression.

Specifically, the DAHP level in LC224 was much lower compared to QP478, which may suggest the aroB activity in LC224 driven by tac promoter was higher than that of QP478. Except for DAHP, LC224 accumulated a higher amount of metabolites in the shikimate pathway and fewer metabolites in the EDEMP cycle relative to QP478 (FIG. 5a, b), suggesting greater flux entering the shikimate pathway in LC224 and enabling greater muconate biosynthesis.

Although its precursor 3DHQ was not detected in any samples, quinic acid (quinate, QA) was substantially accumulated in LC224 (FIG. 5b), which may suggest an overflow of carbon resulting from overexpression of aroB. Shikimic acid (shikimate, SA) accumulation in LC224 is evidence of muconate biosynthesis through the aroE pathway, while SA accumulation was much lower in QP478 relative to LC224, which may be related to the aroK duplication in QP478. The accumulation of anthranilic acid in the culture media likely represents another case of overflow metabolism. More catechol (CAT) accumulated in LC224 (FIG. 5b), which could represent new bottlenecks associated with increased flux to muconate. Together, these results illustrate that engineering to generate LC224 broadly recapitulated the evolved strain QP478 and suggest additional targets for further improvement.

### DISCUSSION

Technologies for the biological production of renewable and sustainable chemicals are greatly needed to displace incumbent petrochemicals and enable a bioeconomy. Critical to this endeavor is the engineering of strains to convert lignocellulosic sugars such as glucose and xylose to product at high titer, rate, and yield. In this work, the maximum theoretical molar yield of muconate from a mixture of glucose and xylose increased from ˜40% to 50%, when the xylose content in the mixture is between 33% and 40% (mol %)—which is a relevant ratio in lignocellulosic hydrolysates (FIG. 1b). This was achieved by introducing the D-xylose isomerase pathway to supply E4P, and reintroducing pgi-1 to enable the EDEMP cycle. ALE was used to identify additional targets to engineer a strain that ultimately achieved a 46% yield on a mixture of glucose and xylose (FIG. 4c), considerably higher than the 35.6% we had achieved previously with a strain engineered to convert glucose alone.

During ALE, two mutations in xylE, A62V and A455V, arose that improved growth on xylose (FIG. 2c). Both of these amino acids are located in the transmembrane domains, but the reasons for this growth improvement remain unclear. Moreover, increased expression of PP_2569, a putative MFS transporter, enabled by a G to A point mutation in the promoter region, led to substantially higher muconate yield and lower biomass yield in LC100 (FIG. 2c), which could reflect a metabolic flux redirect from EDEMP cycle to the shikimate pathway for muconate biosynthesis. Investigation into the function of PP_2569 and the two mutations in XylE is ongoing.

ALE also resulted in a duplication of the genomic region from PP_5050-PP_5242. Within this region, we demonstrated that overexpression of aroB was necessary to reach high growth rates on xylose in LC224. In strain GB062, a strain previously engineered for improved conversion of glucose to muconate by deleting hexR in CJ522,3 transcriptomics indicated that expression of aroB was already increased upon deletion of hexR. In another study in which P. putida KT2440 was engineered to produce PCA from glucose, overexpression of aroB did not contribute to improved production.48 In strain LC100 cultivated on a mixture of glucose and xylose, however, AroB activity seemed to be rate limiting, since overexpression of aroB in LC224 improved growth and muconate production (FIG. 3d, f). This may indicate that with xylose entering the non-oxidative pentose pathway, the supply of E4P was enhanced, leading to improved flux of carbon towards the shikimate pathway by the condensation of E4P and PEP to DAHP. The improved level of DAHP made the next reaction, catalyzed by AroB, rate limiting, where it was not before. Overall, the engineering strategy shown here to improve flux of carbon into and through the shikimate pathway could be leveraged to improve production of other shikimate-derived products from glucose and xylose in P. putida.

Our rationally engineered strain LC224 outperformed the evolved strain QP478 in growth on xylose (FIG. 3d). One potential reason could be the redundancy, complexity, and burden of the large duplication in QP478. Such duplications are likely enabled by recombination within similar sequences at two or more locations within the genome such that duplication of certain regions is favored or limited, ultimately limiting the ability of evolution to arrive at ideal outcomes within laboratory time scales. Genome engineering, however, can be used to make precise changes. Indeed, overexpression of aroB alone in LC224 outperformed QP478 (FIG. 3d-f), which contains the entire PP_5050-PP_5242 duplication. This demonstrates the utility and power of ALE as a tool to identify targets for rational engineering.

Overexpression of aroB substantially improved the sugar utilization of LC224 relative to LC100 (FIG. 2d, 3h). In metabolomic analysis, the intracellular DAHP level of LC224 is much lower than the other two strains (FIG. 5). This may suggest that DAHP buildup has some negative effect on sugar metabolism, which can be relieved by aroB overexpression. Moreover, aroB overexpression alone in QP328, generating LC349, led to slightly lower muconate yield to LC224 on mixture of glucose and xylose. Since restoring the G□A mutation in PPP_2569 in strain QP478 did not have comparable effect to reverse engineering in LC091 in terms of varying the muconate yield (FIG. 2c), these results together may suggest that aroB overexpression (duplication) and upregulation of PP_2569 have similar effect on improving muconate yield.

Metabolomic analysis of our engineered strains provides early insights into future engineering efforts for further improving muconate production, beyond what we demonstrated here with LC224, which will be pursued in future studies. The quinate accumulation by this strain (FIG. 5b) may suggest an approach to improve its performance by overexpressing aroQ or deleting quiA. As for shikimate accumulation, overexpression of aroB and aroK did not improve performance of LC168 relative to LC224, the equivalent strain overexpressing aroB alone (FIG. 3h). This may suggest potential bottleneck(s) in the downstream steps of the pathway, especially considering the relatively high level of extracellular anthranilic acid (ANA) accumulated by QP478 (FIG. 5b). Insufficient conversion of chorismate (CSA) to 4-hydroxybenzoate (4HB) might be one cause of anthranilic acid accumulation. The gene ubiC-C22 encoding chorismate pyruvate-lyase, which was previously engineered to reduce product inhibition, was driven in our strains by the relatively weak lac promoter, and a potential approach to accelerate muconate biosynthesis via shikimate in LC224 could be to overexpress aroK while simultaneously increasing the expression level of ubiC-C22.

Previously conducted technoeconomic analysis for the conversion of glucose as well as glucose and pentose sugars3 indicated that the minimum selling price (MSP) of muconate would decrease substantially with increased yield and rate. Our engineered strain GB271 produced muconate from glucose at a 36% yield and a rate of 0.21 g/L/h, which corresponds with an MSP around $3/kg according to this model.32 Here, LC224 achieved a nearly 50% yield at 0.28 g/L/h (FIG. 4b). This would reduce the MSP to around $2.2/kg, which is close to the $1.96 MSP previously predicted to be commercially viable. The model suggests that the MSP can be further reduced by increasing the rate and yield. Considering the 50% molar yield is already at the theoretical maximum in our strain design, further rate increases will be key to cost improvements.

In conclusion, this work demonstrates an effective strategy for producing muconate from glucose and xylose using P. putida. Considering the exceptionally high yield and titer of muconate from glucose and xylose, our strain LC224 could also represent a promising platform strain for the production of other shikimate pathway-derived compounds.

Methods

Plasmid construction. Q5® High-Fidelity 2× Master Mix (New England Biolabs) was used for all polymerase chain reactions, followed by DpnI (New England Biolabs) digestion to remove the cell-derived plasmid template when necessary. NEBuilder HiFi Assembly Master Mix (New England Biolabs) was used for plasmid construction followed by transformation into chemically competent NEB 5-α F′Iq E. coli (New England Biolabs), or into CopyCutter™ EPI400™ electrocompetent E. coli to increase plasmid yield, all according to manufacturer's instructions. Transformants were selected on Lysogeny Broth (LB) agar (Lennox) plates supplemented with 50 μg/mL kanamycin (Sigma-Aldrich) and grown overnight at 37° C. Correct constructs were confirmed by Sanger sequencing (GENEWIZ, Inc.). Some plasmids were purchased already synthesized (Twist Bioscience).

Strain construction. Gene deletions, insertions, and replacements were performed as previously described49, using the kanamycin-resistant gene nptII as a selection marker for the first-round homologous recombination of the plasmid into the chromosome, and the sucrose-sensitive gene sacB as the counterselection marker for the second round of homologous recombination out of the chromosome. Correct gene deletions, insertions, and replacements were identified by diagnostic colony PCR product based on differences in product size or presence using MyTaq™ Red Mix (Bioline). For point mutation insertions and restorations, correct replacements were screened by comparing the intensity of the bands of the colony PCR products using primers of which the 3′ nucleotide annealed to the mutated nucleotide, followed by confirmation with Sanger sequencing (Genewiz).

Plasmids were transformed into P. putida via either electroporation or conjugation. For electroporation, electrocompetent cells were prepared by washing in 300 mM sucrose according to the previously described method,50 using the backbone plasmid pK18sB,51 as previously described.32 For conjugation, plasmids were built based on backbone plasmid pK18msB which contains the R4P oriT and were transferred from donor E. coli S17-1 cells52 to the recipient P. putida strains. The plasmid-containing donor E. coli S17-1 strain was inoculated into LB medium supplemented with 50 μg/mL kanamycin and cultivated in a shaking incubator at 37° C., 225 rpm overnight; the recipient P. putida strain was inoculated into LB medium and cultivated in a shaking incubator overnight at 30° C., 225 rpm. Subsequently, 1 mL overnight donor and recipient cells were centrifuged at 5000 g for 2 min and washed with LB medium twice, then the pellets were resuspended and 400 μL of each were mixed in one microcentrifuge tube and centrifuged again. The mixed pellets were resuspended using 50 μL LB medium and dropped onto a LB plate with two antibiotics at low concentration: 10 μg/mL chloramphenicol to inhibit cell growth of E. coli S17-1, and 5 μg/mL kanamycin to inhibit cell growth of P. putida. The plate was incubated at 30° C. for 6-10 hours to allow for conjugation, after which the cells were streaked for single colonies on the same plate and incubated at 30° C. overnight. Single colonies on the LB plate containing low concentration antibiotics were restreaked to a new LB plate with 100 μg/mL chloramphenicol, which P. putida is naturally resistant to, and 50 μg/mL kanamycin, to select for transconjugants. All strains used in this study are described in Table 1.

Shake flask and plate reader experiments. For shake flask and growth curve experiments, seed cultures were inoculated from glycerol stocks into a 14 mL round bottom Falcon® tubes containing 5 mL of LB Miller medium and incubated overnight at 30° C. and 225 rpm. Overnight cultures were then inoculated into a 125 mL baffled shake flask containing 10 mL LB medium to an initial OD600 of 0.2. These second seed cultures were cultivated at 30° C. at 225 rpm for 4 hours to reach an OD600 of ˜2. The second seed cultures were washed twice with M9 salts (6.78 g/L Na2HPO4, 3 g/L KH2PO4, 0.5 g/L NaCl, 1 g/L NH4Cl) and then inoculated into a 125 mL baffled shake flask containing 25 mL modified M9 minimal medium (6.78 g/L Na2HPO4, 3 g/L KH2PO4, 0.5 g/L NaCl, 1 g/L NH4Cl, 2 mM MgSO4, 100 μM CaCl2), 18 μM FeSO4) supplemented with either 30 mM xylose or 30 mM glucose and 15 mM xylose, to an initial OD600 of 0.1. The molar ratio of glucose and xylose in mixed substrates is 2:1, which is the ratio typical of corn stover hydrolysates. All growth curves were characterized on Bioscreen C MBR analyzers (Growth Curves US) using 300 μL cultures inoculated as described for the shake flasks above. Absolute growth rate (μA) and growth lag (λ) were calculated based on Gompertz equation using the code deposited in Github: https://github.com/scott-saunders/growth_curve_fitting. All details of the growth curve parameters calculation, including absolute growth rate (μA) and growth lag (λ).

The pH values of flasks were monitored at each sampling time point using a mini pH meter (HORIBA LAQUAtwin pH-33), and when necessary, 1N NaOH was used to adjust the pH to 7. For shake flask experiments, yields were calculated at the timepoint where maximum muconate concentration was detected, as the amount of muconate (in moles) produced divided by the glucose and xylose (in moles) utilized.

Metabolic modeling. A previously developed core-carbon metabolic model3 was extended with reactions from the aroE synthetic route, using stoichiometry adapted from a genome-scale model of P. putida KT2440.54 Yield calculations were performed assuming no ATP maintenance requirement by optimizing the muconate flux while varying the xylose fraction in the medium. Calculations were performed using the cobrapy library in Python.55

Adaptive laboratory evolution (ALE) with xylose as the sole carbon source. To conduct ALE, strain QP328 was first inoculated in LB Miller medium from a glycerol stock and grown overnight as a seed culture. Cells from the overnight culture were then washed with M9 salts twice and resuspended in M9 salts. The washed cells were inoculated to an initial OD600 0.1 in a culture tube containing 5 mL M9 medium with 10 mM xylose as a sole carbon source and cultivated at 30° C. with shaking at 225 rpm. Serial passaging was performed by transferring 1% (vol/vol) of the culture into fresh medium when growth was observed, which initially took up to around two weeks of cultivation. The number of generations was calculated based on the OD600 value, according to the formula: number of generations=ln(ODfinal/ODinitial)/ln(2). Initial, periodic intermediate, and final populations were preserved as glycerol stocks.

Quantitative reverse transcription (RT-qPCR). For RT-qPCR of PP_2569 in strains LC091 and LC100, cells were cultivated in shake flasks with M9 and 30 mM xylose according to the shake flasks protocol mentioned above. For RT-qPCR of PP_4302 in strains LC100 and LC224, cells were cultivated in shake flasks with LB medium. Cells were harvested in mid-log phase, and were broken using TRI® Reagent (Sigma, T9424), followed by RNA miniprep using Direct-Zol™ RNA miniprep kit (Zymo Research, R2052) following the protocol. The extracted total RNAs were then digested using DNase I (Zymo Research, E1009-A) and purified using RNA Clean & Concentrator™-5 kit (Zymo Research, R1014). RNA concentrations were determined using NanoDrop™ one (Thermo Scientific) at 260 nm, 500 ng total RNA of each sample were added for reverse transcription, using the iScript Reverse Transcription Supermix (Bio-Rad), to synthesize cDNA. RT-qPCR was conducted using KiCqStart® SYBR® Green qPCR ReadyMix (Sigma-Aldrich), with the 7500 Fast Real-Time PCR System (Applied Biosystems).

Housekeeping gene rpoD was employed as a reference control to normalize different samples.58 Primers oLC-0109 and oLC-0110 were used for amplifying rpoD; oLC-0107 and oLC-0108 were used for amplifying PP_2569; oLC-0353 and oLC-0354 were used for amplifying PP_4302. We used the 2-ΔΔCt method to quantify transcriptional levels between samples.

Bioreactor cultivations. To evaluate strains QP478 and LC224 in bioreactors, the strains were inoculated from glycerol stocks in 250 mL baffled flasks containing 50 mL of LB (Miller) and incubated at 30° C. and 225 rpm for 16 h. Seed cultivations were conducted in duplicate for each strain and each replicate was utilized to inoculate independent bioreactors. The cells were centrifuged (5000 g, 10 min), the supernatant was discarded, and the cells resuspended in 5 mL of modified M9. The modified M9 contained 13.56 g/L Na2HPO4, 6 g/L KH2PO4, 1 g/L NaCl, 2 g/L (NH4)2SO4, 2 mM MgSO4, 100 μM CaCl₂), and 36 μM FeSO4.

Cells were inoculated in 0.5 L bioreactors (BioStat-Q Plus, Sartorius Stedim Biotech) at an initial OD600 of 0.2. The batch phase consisted of growth on 300 mL of modified M9 with 10.6 g/L glucose and 4.4 g/L xylose, which mimics the sugar ratio in sugar hydrolysates from corn stover.53 The fed-batch phase was initiated when the sugar concentration was approximately 7 g/L, at which point sugars were fed to maintain sugar concentrations between ˜2-10 g/L by manually modifying feeding rates. The feeding solution contained 353 g/L glucose and 147 g/L of xylose, and its pH was adjusted to pH 7 with NaOH. The bioreactors were controlled at pH 7 by the addition of 4N NH40H, at 30° C., and air was sparged at 1 vvm. The initial agitation speed in the batch phase was 350 rpm. When the dissolved oxygen (DO) reached a value of 30%, it was automatically controlled at that level by automatic agitation adjustments. Samples were taken periodically to evaluate bacterial growth and to analyze sugar concentrations and muconate.

Muconate, glucose, and xylose analyses. cis,cis- and cis,trans-muconic acid isomers were analyzed as described previously56 and quantitation was achieved using the chromatographic conditions below. Samples and standards were analyzed using Agilent 1290 series UHPLC (Agilent Technologies) coupled with a diode array detector (DAD) and a Phenomenex Luna C18 (2) 5 μm, 4.6×150 mm column. An injection volume of 1 μL was injected onto the column in which the temperature was held constant at 45° C. Muconic acid isomers were monitored and quantified at the wavelength 265 nm with a quantitation range of 1 ppm to 500 ppm. A gradient of 0.16% formic acid in water (A) and acetonitrile (B) was utilized at a flow rate of 0.5 mL/min. Chromatographic separation of analytes was attained using the following gradient program: initial (t0) to t=1 min: A-100% and B-0%; ramp to A-50% and B-50% from t=1-7.67 min; ramp to A-30% and B-70% from t=7.67-9.33 min and held until 10.67 min. At 10.68 min, the gradient was returned to A-100% and B-0% and maintained isocratic for a total run time of 13 min. A calibration verification standard was run every 10-15 samples to ensure detector stability. Glucose was quantified by HPLC as described previously57 and xylose was similarly quantified using HPLC with refractive index detection coupled with an Aminex HPX-87H column (Bio-Rad). Pure standards were purchased from Sigma-Aldrich.

Metabolomic analysis. Cell pellets and supernatant samples were collected separately by centrifugation of 1 mL of shake flask cultures grown on glucose and xylose at the mid-log phase (OD600˜1.0), and the samples were frozen at −80° C. until analysis. Cell pellets were lyophilized, and the intracellular metabolites were extracted from 3 mg of dried biomass using a solvent mixture of chloroform/methanol/water,60 and both aqueous and organic layers were transferred to a new vial and dried completely. The extracellular metabolites were prepared by drying the supernatant samples before the analysis. The metabolites were chemically derivatized based on the method reported previously. To protect carbonyl groups and reduce the number of tautomeric isomers, methoxyamine (20 μL of a 30 mg/mL stock in pyridine) was added to each sample, followed by incubation at 37° C. with shaking for 90 min. To derivatize hydroxyl and amine groups to trimethylsilylated (TMS) forms, N-methyl-N-(trimethylsilyl) trifluoroacetamide with 1% trimethylchlorosilane (80 μL) was added to each vial, followed by incubation at 37° C. with shaking for 30 min. The samples were allowed to cool to room temperature and were analyzed by gas chromatography-mass spectrometer (GC-MS) coupled with a HP-5MS column (30 m×0.25 mm×0.25 μm; Agilent Technologies) was used for untargeted analyses. Samples (1 μL) were injected in splitless mode, and the helium gas flow rate was determined by Retention Time Locking function based on analysis of deuterated myristic acid (Agilent Technologies, Santa Clara, CA). The injection port temperature was held at 250° C. throughout the analysis. The GC oven was held at 60° C. for 1 min after injection, and the temperature was then increased to 325° C. by 10° C./min, followed by a 10 min hold at 325° C. Data were collected over the mass range 50-600 m/z. A mixture of FAMEs (C8-C28) was analyzed together with the samples for retention index alignment purposes during subsequent data analysis. GC-MS data files were converted to CDF format, and they are deconvoluted and aligned by Metabolite Detector.62 Identification of metabolites was done by matching with PNNL metabolomics databases—augmented version of Fiehn database.63 The database contains mass spectra and retention index information of over 1,000 authentic chemical standards and they were cross-checked with commercial GC-MS databases such as NIST20 spectral library and Wiley 11th version GC-MS databases.64, 65 Three unique fragmented ions were selected and used to integrate peak area values, and a few metabolites were curated manually, when necessary.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all molecular weight or molecular mass values given for nucleic acids or polypeptides are approximate, and are provided for description. Although methods and materials similar or equivalent to those described herein can be used in the practice or testing of this disclosure, suitable methods and materials are described below. The term “comprises” means “includes”. In addition, the materials, methods, and examples are illustrative only and not intended to be limiting.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Those skilled in the art will understand that the genetic alterations, including metabolic modifications exemplified herein, are described with reference to a suitable host microorganism such as species from the Pseudomonas genus and their corresponding metabolic reactions or a suitable source microorganism for desired genetic material such as genes for a desired metabolic pathway. However, given the complete genome sequencing of a wide variety of microorganisms and the high level of skill in the area of genomics, those skilled in the art will readily be able to apply the teachings and guidance provided herein to essentially all other microorganisms. For example, the Pseudomonas metabolic alterations exemplified herein can readily be applied to other species by incorporating the same or analogous encoding nucleic acid from species other than the referenced species. Examples of other species include Sphingobium species (sp.) SYK-6, Rhodococcus jostii, Cupriavidus necator, Acinetobacter sp. ADP1, Amycolatopsis sp. ATCC 39116, E. coli, S. cerevisae, and/or fungi. Such genetic alterations include, for example, genetic alterations of species homologs, in general, and in particular, orthologs, paralogs or nonorthologous gene replacements.

Disclosed herein are methods for the integrated production of fuels, chemicals or materials from biomass, including lignin, cellulose, and hemicellulose, via catabolic pathways in bacteria. These methods enable a biological funneling approach for heterogeneous aromatic streams, thus opening a new route to produce renewable chemicals and fuels from biomass. Methods to couple this biological funneling to upstream lignin, cellulose, and/or hemicellulose depolymerization and downstream catalytic upgrading processes, thereby enabling a versatile, general approach to valorize lignin are also disclosed.

The methods presented herein may include the steps of lignin, cellulose, and/or hemicellulose depolymerization, biological funneling to a desired intermediate, followed by recovery and transformation to a value-added product. There is significant versatility in each step of this process such that it can be adapted to various feedstocks (e.g. raw materials), unit operations, and targeted fuel and chemical portfolios.

FIG. 1a illustrates an exemplary biorefinery 100 that utilizes lignocellulosic biomass 110 to produce a high volume, commodity chemical, in this case adipic acid. FIG. 1a illustrates corn and/or corn stover as the lignocellulosic biomass 100 raw material (the carbon, oxygen, and hydrogen source). However, other examples of lignocellulosic biomass 110 that may be processed by such a biorefinery 100 include wheat straw, bagasse, wood, wood chips, bark, grass, municipal solid waste, and any other common and/or high volume biomass source characterized by a high content of cellulose, hemicellulose, lignin, or combinations thereof. In some embodiments, the biomass utilized may include at least one of wood, wood chips, bark, sawdust, wood pellets, wood briquettes, forestry waste, pine, poplar, willow, Eucalyptus, Nothofagus, sycamore, ash, miscanthus, switchgrass, reed canary grass, rye, giant reed, hemp, bamboo, sugar cane, bagasse, corn, corn stover, wheat, wheat straw, sugar beets, sorghum, rapeseed, waste vegetable oil, palm oil, algae, municipal solid waste, and/or yard clippings.

The biomass feed stream may be provided by mechanical conveyor and/or pneumatically. One skilled in the art will recognize that some preprocessing of the biomass may be required to enable the providing step. Examples of preprocessing include size reduction, screening, filtering, washing, and combinations thereof. Size reduction may be accomplished by chopping, cutting, grinding, and combinations thereof, using for example, a hammer-mill and/or knife-mill.

After receiving the lignocellulosic biomass, FIG. 1a illustrates that one or more pretreatment-fractionation operations 120 may pretreat and then separate the lignocellulosic biomass 110 into its component building blocks; e.g. lignin, cellulose, and hemicellulose. FIG. 1a also illustrates that the polysaccharide components may be separated from a main processing stream, resulting in a lignin-rich stream. The polysaccharides may then be directed to separate and independent processing operations for conversion to, for example, sugars, chemicals, and/or fuels by dedicated processing operations. The lignin stream may be subsequently directed through various processing operations that may include lignin depolymerization operations 130 and microbial catalysis operations 140, resulting in the production of useful chemical intermediates, for example muconic acid. The intermediate chemicals produced may then be processed through one or more separation/purification operations 150 to yield purified intermediate components (e.g. muconic acid), after which the intermediates may be packaged and/or stored for delivery and sale, or directed to down-stream processing, for subsequent upgrading to higher value chemicals and/or fuels. FIG. 1a illustrates an exemplary upgrading operation that includes catalytic hydrogenation 160, to convert muconic acid to adipic acid. Thus, biological funneling of lignin-derived monomers by engineered microorganisms may be combined with downstream upgrading to facilitate the development of an immense range of products.

Any pretreatment, fractionation, or depolymerization method that generates a lignin-containing stream is suitable for use in the methods described herein. Referring again to FIG. 1a, pretreatment and/or fractionation operations 120 of a lignocellulosic biomass 110 such as corn stover may include contacting the lignocellulosic biomass with an alkaline compound or biological or chemical catalyst to generate a soluble lignin-containing stream. Exemplary processing conditions include treatment with a base such as sodium hydroxide for a time ranging from several minutes to several hours at a temperature ranging from about 50° C. to about 200° C. at a solids loading of about 5 wt % to about 10 wt % solids in a mixer. These conditions may be varied by one skilled in the art, commensurate with the biomass source, the chemical or biological catalyst used, and other process and physical property parameters. Some embodiments of the present invention include lignin-polysaccharides separations using separation methods known to one skilled in the art. Some examples of lignin-polysaccharide separation methods which may be used in the present invention are described in U.S. Pat. Nos. 5,730,837; 2,037,001; 1,594,389; 1,888,025; 2,042,705; 3,932,207; 4,520,105; and 4,594,130, all of which are incorporated herein by reference in their entirety. Should definition discrepancies arise between publications incorporated herein by reference and the present written description and claims, the definitions provided herein shall over rule any other external definitions.

Referring again to FIG. 1a, lignin depolymerization operations 130 to low molecular weight aromatics may be achieved via thermal, biological, and/or catalytic processing. For example, white rot fungi and some bacteria may depolymerize lignin to its monomeric constituents using powerful oxidative enzymes. Lignin is composed of three monomeric phenylpropanoid units that differ in their degree of methoxylation, which are polymerized by carbon-carbon and carbon-oxygen bonds formed during lignin biosynthesis via radical coupling reactions. Thus, lignin depolymerization operations 130 may include a broad range of catalytic, thermal, and biological routes, and may yield a chemically heterogeneous pool of depolymerization intermediates, which may include for example, p-coumaric acid, ferulic acid, benzoic acid, phenol, coniferyl alcohol, caffeic acid, vanillin, and/or 4-hydroxybenzoic acid.

Referring again to FIG. 1a, microbial catalysis 140 may include biological funneling of one or more lignin depolymerization products/intermediates to produce molecules from acetyl-CoA, the tricarboxylic acid cycle, and beyond in carbon metabolism. In aerobic systems, a microbial catalysis operation 140 may utilize aromatic-catabolizing microorganisms that involve the use of “upper pathways”. These “upper pathways” may employ a diverse battery of enzymes to funnel aromatic monomers and oligomers to key central intermediates, such as catechol and protocatechuic acid. From these central intermediates, dioxygenase enzymes may oxidatively cleave carbon-carbon bonds in the aromatic rings to produce ring-opened species that may then be “funneled” to central carbon metabolism routes, which may then ultimately lead to the tricarboxylic acid cycle. These catabolic pathways may enable engineered microorganisms to utilize a broad range of both natural and xenobiotic aromatic molecules as carbon and energy sources. In terms of chemicals and/or biofuels production, these metabolic pathways offer a direct, powerful means to biologically ‘funnel’ the heterogeneous slate of molecules produced from lignin depolymerization into either fuels or chemicals. An example of a useful intermediate chemical resulting from a microbial catalysis operation 140 (via biological funneling), as described above, is muconic acid.

The separation/purification operations 150 of the fuels, chemicals, and/or intermediates resulting from the microbial catalysis operation 140 may include a variety of unit operations. The selection of one or more specific unit operations for the separation/purification operation 150 will depend on the on the design criteria and operating conditions of the upstream processing; e.g. type of lignocellulosic biomass 110 utilized, and pretreatment-fractionation operation 120 efficiency and yield, and the species targeted for removal from the lignin stream. The separation/purification operation 150 needed may also depend on the details of the microbial catalysis operation 140; e.g. type of microorganism used, culture broth composition, etc. However, examples of unit operations for the separation/purification operation 150 may include filtration, centrifugation, distillation, vacuum distillation, adsorption, membrane separations, cross-flow membrane filtration, crystallization, and/or any other suitable separation/purification unit operations. For example, culture broth containing muconic acid from microbial catalysis 140 may be centrifuged and/or filtered to produce a solids stream (e.g. liquid stream with a relatively high concentration of cell matter) and a substantially solids-free liquid stream containing muconic acid. The muconic acid containing stream may then be treated by one or more unit operations, e.g. adsorption, crystallization, to produce a relatively pure muconic acid stream capable of downstream upgrading to value-added final products (e.g. chemicals, fuels, etc.)

Alternatively, a biorefinery 100 may also be designed to utilize predominantly the cellulosic components of the lignocellulosic biomass 110 to produce useful fuels and chemicals, as illustrated in FIG. 1b. In this example, lignin may be separated from the polysaccharides to form a polysaccharide-rich stream. The lignin may then be directed to separate and independent processing operations for conversion to lignin depolymerization products, for example aromatic compounds. The polysaccharide stream may be subsequently directed through various processing operations that may include polysaccharide depolymerization operations 135 and microbial catalysis operations 140, resulting in the production of useful chemical intermediates, for example muconic acid. The microbes (microorganisms) utilized for the microbial catalysis operation 140 may be engineered in this example to funnel polysaccharide degradation products (e.g. glucose, xylose, arabinose) to protocatechuic acid and/or catechol and subsequently to muconic acid. The muconic acid containing stream from the microbial catalysis operation 140 of FIG. 1b, may then be subsequently separated and/or purified in a separation/purification operation 150 operations. These operations for polysaccharide-based products may be significantly different from the separation/purification operations 150 utilized for the processing of the lignin-based products of FIG. 1a, because the microorganisms engineered to metabolize lignin depolymerization products may be significantly different from the microorganisms engineered to metabolize polysaccharide depolymerization products, as may be the culture media/broth and culture conditions. Thus, another biorefinery example may begin with something like the biorefinery of FIG. 1a, where the polysaccharide stream removed by the pretreatment-fractionation 120 operations may be fed to a polysaccharide depolymerization operation 130 operation like that of FIG. 1b. In so doing, both the polysaccharide stream and the lignin stream may have their own dedicated depolymerization operation, microbial catalysis operation, separation/purification operation, and upgrading operation (e.g. catalytic hydrogenation).

Alternatively, one or more microorganisms may be engineered to process both lignin depolymerization products and polysaccharide depolymerization products in a single microbial catalysis step. This option is illustrated in FIG. 1c. Thus, FIG. 1c combines FIG. 1a and FIG. 1b into a single series of operations wherein removal of a constituent biomass component (e.g. lignin or polysaccharides) does not occur. Instead, any pretreatment-fractionation steps (not shown) may be limited to, for example, washing steps for removing debris, metal removal steps, etc. Thus, biomass depolymerization 137 of lignin and cellulose/hemicellulose may occur simultaneously in a single processing step. Similarly, microbial catalysis 140 of the resultant lignin and polysaccharides depolymerization products may occur simultaneously in a single processing step (e.g. in a single bioreactor). Simultaneous culturing of the lignin and polysaccharides depolymerization products may include one biocatalyst/microorganism that has been engineered to process both lignin and polysaccharide depolymerization products to produce useful products (e.g. muconic acid). Alternatively, one or more biocatalysts/organisms may be engineered and/or optimized for the conversion of lignin depolymerization products to useful chemical intermediates and one or more additional biocatalysts/microorganisms may be engineered and/or optimized for the conversion of polysaccharides to useful chemical intermediates, with the various microorganisms used together in one or more culturing steps. Once the microbial catalysis 140 produces muconic acid, the resultant culture broth containing the muconic acid will be treated in a separations/purification operation 150 to produce a muconic acid stream of sufficient purity and quality to allow subsequent upgrading 170 (e.g. hydrogenation) to final products; e.g. adipic acid and/or nylon-6,6.

This disclosure will focus next on details and examples regarding the microbial catalysis 140 portion of the biorefinery 100 and will return later to the separation/purification operation 150 and the upgrading 170 portions of the biorefinery 100.

### Microbial Catalysis

Microorganisms engineered and/or modified to funnel lignin depolymerization products and/or polysaccharide depolymerization products to useful molecules (e.g. chemicals and/or fuels) may include prokaryotes such as bacteria or eukaryotes such as yeasts or fungi. Further examples include Pseudomonas sp., Sphingobium sp. SYK-6, Rhodococcus jostii, Cupriavidus necator, Acinetobacter sp. ADP1, Amycolatopsis sp. ATCC 39116, E. coli, S. cerevisae, bacterial species from the genera Streptomyces or Bacillus, and/or fungi. Another example of a genetically modified bacterium may include the genus Pseudomonas. In some cases, the genetically modified microorganism may include at least one Pseudomonas species such as aeruginosa, chlororaphis, fluorescens, pertucinogena, putida, stutzeri, syringae, and/or incertai sedis. In other cases, the genetically modified microorganism may include at least one of P. putida, P. fluorescens, and/or P. stutzeri. In still other cases, the genetically modified microorganism may include at least one strain of Pseudomonas putida KT2440.

In some embodiments, the genetically modified microorganism may include at least one of P. putida group, including at least one of P. cremoricolorata, P. entomophila, P. fulva, P. monteiii, P. mosselii, P. oryzihabitans, P. parafulva, P. plecoglossicida, and/or P. putida. In some embodiments, the genetically modified microorganism may include at least one of the P. fluorescens group, including at least one of P. antarctica, P. azotoformans, P. brassicacearum, P. brenneri, P. cedrina, P. corrugate, P. fluorescens, P. gessardii, P. libanensis, P. mandelii, P. marginalis, P. mediterranea, P. meridian, P. migulae, P. mucidolens, P. orientalis, P. panacis, P. protegens, P. proteolytica, P. rhodesiae, P. synxantha, P. thivervalensis, and/or P. tolaasii.

As used herein, “exogenous” refers to something originating from another genetic source or species. So, as used herein, a genetically modified bacterium refers to a bacterium wherein the genetic modification is either the addition of genetic material from another species and/or the deletion of a portion of its own endogenous genetic material. So, as used herein, “endogenous” refers to native or naturally occurring genetic material of the microorganism itself.

As used herein, “gene” and “genetic source” and “genetic material” refer to a segment of nucleic acid that encodes an individual protein or RNA molecule (also referred to as a “coding sequence” or “coding region”) and may include non-coding regions (“introns”) and/or associated regulatory regions such as promoters, operators, terminators and the like, that may be located upstream or downstream of the coding sequence.

Genetic modifications and/or engineering to a microorganism to enable and/or improve the funneling of lignin depolymerization products and/or polysaccharide depolymerization products to useful intermediate compounds and/or useful fuels and/or chemicals may include at least one exogenous gene addition, at least one endogenous gene deletion, and/or the over-expression of at least one endogenous and/or exogenous gene. Such additions may be genomic and/or include the addition of plasmids that contain the desired gene.

FIG. 2 illustrates exemplary engineered pathways for enabling an engineered microorganism to metabolize at least one of lignin, cellulose, hemicellulose, lignin depolymerization products, cellulose depolymerization products, and/or hemicellulose depolymerization products, to produce muconic acid. In this example, a strain of P. putida may include pathways for metabolizing lignin depolymerization products (e.g. aromatic compounds) and for metabolizing polysaccharide depolymerization products (e.g. glucose, xylose, arabinose, mannose, galactose, and/or rhamnose) to maximize the production of muconic acid. Referring again to FIG. 2, the exemplary genetically modified microorganism may convert coniferyl alcohol to coniferyl aldehyde and/or the genetically modified microorganism may convert the coniferyl aldehyde to ferulic acid and/or the genetically modified microorganism may convert the ferulic acid to at least one of vanillin and/or vanillic acid. A genetically modified microorganism may convert the vanillin to vanillic acid and/or the genetically modified microorganism may convert the vanillic acid to protocatechuic acid. A genetically modified microorganism may convert p-coumaric acid to 4-hydroxybenzoic acid and/or the genetically modified microorganism may convert 4-hydroxybenzoic acid to protocatechuic acid. A genetically modified microorganism may convert at least one of glucose, xylose, and/or arabinose to 3-dehydroshikimate (DHS) and/or the genetically modified microorganism may convert DHS to protocatechuic acid. A genetically modified microorganism may convert protocatechuic acid to catechol. A genetically modified microorganism may convert phenol to catechol. A genetically modified microorganism may convert benzoic acid to benzoic acid diol. A genetically modified microorganism may convert benzoic acid diol to catechol. A genetically modified microorganism may convert the catechol to muconic acid. In some examples, a genetically modified microorganism may be engineered to complete one or more of the reactions described above.

FIG. 2 illustrates that various genes coding a number of different enzymes may be manipulated, for example removed, added, over-expressed, and/or under-expressed to genetically engineer a microorganism (e.g. P. putida) to maximize the yield and/or selectivity of the target product molecule (e.g. muconic acid). For example, a microorganism may be engineered such that one or exogenous genes may be added to the microorganism. For example, a microorganism may be engineered such that one or more genes encoding exogenous decarboxylases may be added to the microorganism. A microorganism may be engineered such that one or more genes encoding exogenous monooxygenases may be added to the microorganism. A microorganism may be engineered such that one or more exogenous genes encoding dehydratases may be added to the microorganism. A microorganism may be engineered such that one or more genes encoding one or more exogenous monooxygenases, decarboxylases, and/or dehydratases may be added to the microorganism. Gene additions may be genomic additions or through the use of plasmids. Genes may be provided by any suitable and compatible microorganism for example Enterobacter cloacae, Pseudomonas sp., and/or Bacillus cereus. A microorganism may be engineered such that one or more genes encoding endogenous demethylases may be over-expressed by the microorganism.

In some embodiments, a microorganism may be engineered for optimized muconic acid production such that one or more endogenous genes may be removed from the microorganism. For example, a microorganism may be engineered such that one or more genes encoding catabolite repression control proteins may be removed, for example the gene encoding the Crc protein. A microorganism may be engineered such that one or more endogenous genes encoding dioxygenases may be removed from the microorganism. A microorganism may be engineered such that one or more endogenous genes encoding muconating lactonizing enzymes may be removed from the microorganism. A microorganism may be engineered such that one or more endogenous genes encoding muconolactone isomerases may be removed from the microorganism. In some embodiments, a microorganism may be engineered such that at least one gene encoding a catabolite repression control protein, a dioxygenase, a muconating lactonizing enzyme, and/or a muconolactone isomerase are removed from the microorganism.

A microorganism may be optimized for muconic acid production by the addition of several genes. For example, an exogenous decarboxylase that may be added to a microorganism may include 3,4-dihydroxybenzoate decarboxylase from Enterobacter cloacae subsp. cloacae (ATCC 13047). Such an exogenous decarboxylase may be encoded by an aroY gene. In some embodiments, an exogenous dehydratase added to a microorganism may be from at least one of Klebsiella pneumoniae, K. oxytoca, K. planticola, K. ornithinolytica, K. terrigena, Enterobacter cloacae, Enterobacter cancerogenus, Enterobacter hormaechei, Enterobacter mori or combinations thereof. In some embodiments, an exogenous dehydratase may be engineered into a microorganism, where the exogenous dehydratase may be encoded by an aroZ gene. A microorganism may be modified by the addition of at least one exogenous monooxygenase encoded by at least one gene of dmpK, dmpL, dmpM, dmpN, dmpO, and/or dmpP from the microorganism Pseudomonas sp. CF600 or the gene pheA from Pseudomonas sp. EST1001. For example, a microorganism may be modified to include the addition of each of dmpK, dmpL, dmpM, dmpN, dmpO, and dmpP, where such a modification is referred to herein as the addition of dmpKLMNOP. In some embodiments, at least one exogenous decarboxylase may be engineered into a microorganism, where the at least one exogenous decarboxylase may further include at least one gene of ecdB and/or ecdD. At least one exogenous dehydratase may be engineered into a microorganism, where the exogenous dehydratase may be encoded by asbF. At least one endogenous demethylase may be over-expressed in an microorganism, where the demethylase may be encoded by at least one of vanA, vanB, or ligM.

A microorganism may be optimized for muconic acid production by the deletion of at least one gene encoding a muconate lactonizing enzyme and/or a muconolactone isomerase, such as catB and/or catC. In some examples, a microorganism may be manipulated to maximize muconic acid production by the removal of at least one dioxygenase, where the dioxygenase may be encoded by at least one gene of pcaH and/or pcaG.

In some examples, at least one endogenous gene deletion to modify a microorganism for improved muconic acid production may include the deletion of at least one gene that encodes at least one enzyme that metabolizes muconic acid to a different molecule. In some embodiments, at least one endogenous gene deletion from a microorganism may include the deletion of at least one gene that encodes at least one enzyme in the β-ketoadipate pathway that metabolizes muconic acid to a different molecule.

In still further embodiments, the modified (e.g. engineered) microorganism may include at least one exogenous gene addition that encodes at least one enzyme of the pentose phosphate pathway and the addition of at least one gene that encodes at least one enzyme of a glycolytic pathway, such as the Embden-Meyerhof-Parnas pathway or the Entner-Doudoroff pathway. For example, some of the exogenous genes that may be added to a microorganism for improved muconic acid production may encode enzymes from the pentose phosphate pathway, such as at least one of glucose-6-phosphate dehydrogenase, gluconolactonase, 6-phosphogluconate dehydrogenase, ribulose-5-phosphate isomerase, ribulose-5-phosphate-3-epimerase, transketolase, and/or transaldolase. As another example, some of the exogenous genes that may be added to a microorganism for improved muconic acid production may encode enzymes from the Glycolysis pathway, such as at least one of hexokinase, phosphofructokinase, fructose-bisphosphate aldolase, triosephosphate isomerase, glyceraldehyde 3-phosphate dehydrogenase, phosphoclycerate kinase, phosphoglycerate mutase, enolase, and/or pyruvate kinase. In further embodiments, the modified microorganism may include at least one endogenous gene deletion where at the gene deleted encodes an enzyme for converting 3-dehydroshikimate (DHS) to an amino acid.

As used herein, the term “homologous” sequences of nucleic acids and proteins refer to sequences that have a statistically significant degree of similarity. In some embodiments of the present invention, any of the genes and the proteins and/or enzymes that they encode, e.g. dehydratases, decarboxylases, dioxygenases, monooxygenases, genes and enzymes from the pentose phosphate pathway, genes and enzymes from the Glycolysis pathway, may include nucleic acid and/or amino acid sequences that are homologous to the specific examples given in that the homologs have nucleic acid and/or amino acid sequences that are at least 70%, 75%, 85%, 90%, 91%, 92%, 93%, 94%, 95%, 96%, 97%, 98%, or 99% homologous to or identical to the sequences of the exemplary enzymes provided above. Nucleic acid sequences that do not show a high degree of identity may nevertheless encode similar amino acid sequences, due to the degeneracy of the genetic code. It is to be understood that changes in nucleic acid sequence can be made using this degeneracy to produce multiple nucleic acid sequences that each encode substantially the same protein.

As a result of at least one of the genetic modifications described above, a genetically engineered microorganism may be used to metabolize at least one of lignin, cellulose, hemicellose, or combinations thereof, to produce useful final products, chemicals, and chemical intermediates. Further, a modified microorganism may be used to metabolize at least one of the intermediations of lignin depolymerization, cellulose depolymerization, hemicellose depolymerization, or combinations thereof, to produce useful final products, chemicals, and chemical intermediates. For example, a modified microorganism (e.g. a bacterial strain such as P. putida) may be engineered to metabolize at least one of carbohydrates, cellobiose, polysaccharides, C5 sugars, C6 sugars, and/or lignin depolymerization products. Sugars that may be metabolized in some embodiments by modified microorganisms described herein may include at least one of xylose, glucose, galactose, arabinose, mannose, or combinations thereof. Lignin depolymerization products that may be metabolized by some embodiments of the modified microorganisms described herein may include least one of phenylpropanoid units, coniferyl alcohol-derived constituents, syringyl structures, coniferyl structures, p-coumaryl groups, or mixtures thereof. In still further embodiments, compounds that may be metabolized by some examples of the modified microorganisms as described herein may include at least one of p-coumaryl alcohol, syringyl alcohol, coniferyl alcohol, coniferyl aldehyde, ferulic acid, feruloyl-CoA, vanillin, vanillic acid, caffeic acid, or mixtures thereof. Other chemical compounds that may be metabolized by some examples of the genetically engineered microorganisms described herein include at least one of 3-dehydroshikimate, p-coumeric acid, 4-hydroxybenzoic acid, phenol, benzoic acid, benzoic acid diol, or mixtures thereof. In some embodiments, a modified microorganism as described herein, engineered to metabolize at least one of lignin, cellulose, hemicellulose, and their respective depolymerization products, may achieve this metabolism by use of at least one of the β-ketoadipate pathway, the Embden-Meyerhof-Parnas pathway, the Entner-Doudoroff pathway, the pentose phosphate pathway, and/or an amino acid synthesis pathway.

Referring again to FIG. 2, several organic acids are shown: e.g. ferulic acid, vanillic acid, caffeic acid, protocatechuic acid, p-coumaric acid, 4-hydroxybenzoic acid, benzoic acid, etc. Organic acids in aqueous solutions will reach an equilibrium concentration/ratio of the conjugate acid (e.g., muconic acid) with the conjugate base (e.g., muconate). The relationships defining these equilibrium concentrations and ratios are controlled at least by the acid dissociation constant and solution pH and often both the acid and base form will be present in an aqueous solution (e.g. culture broth). Thus, for simplification purposes, as used herein reference to either the conjugate acid form (e.g., muconic acid) or the conjugate base form (e.g., muconate) of a molecule means both forms of molecule are present unless specified otherwise.

FIG. 4 summarizes experimental results from an engineered P. putida KT2440 strain. For this example, the strain was modified to capture aromatic species metabolized through protocatechuic acid. This was accomplished by replacing the gene pcaHG encoding a protocatechuate 3,4 dioxygenase with aroY encoding a protocatechuate decarboxylase from Enterobacter cloacae. This enabled the conversion of protocatechuic acid and upstream metabolites to catechol while simultaneously eliminating further catabolism of protocatechuic acid to β-ketoadipate via the TCA-cycle (see FIG. 2, β-ketoadipate not shown). The same P. putida strain was then further engineered to expand substrate utilization and to eliminate further metabolism of muconic acid. This was accomplished be removing CatA and CatB by removing a genomic section containing catR, catBC, and the promoter for catBCA and replacing it with the Ptac promoter, which allowed the constitutive expression of catA. Lastly, as phenol is a commonly derived lignin intermediate, the genes encoding the phenol monooxygenase from Pseudomonas sp. CF600, dmpKLMNOP were integrated into the genome downstream of catA to form an operon driven by the Ptac promoter (see FIG. 3).

The metabolic performance of the engineered P. putida strain (called KT2440-CJ103) was then evaluated in shake-flask experiments to demonstrate substrate utilization and production of muconic acid from model lignin-derived monomers, using acetate as a carbon and energy source. FIG. 4 illustrates the metabolism of benzoate (benzoic acid), p-coumarate, phenol, 4-hydroxybenzoic acid (4-HBA), ferulate (ferulic acid), protocatechuate (protocatechuic acid), vanillin, caffeate (caffeic acid), coniferyl alcohol, and catechol by KT2440-CJ103. KT2440-CJ103 successfully produced muconic acid from catechol, phenol, and benzoate via the catechol branch (see FIG. 2), as well as from protocatechuate, coniferyl alcohol, ferulate, vanillin, caffeate, p-coumarate, and 4-hydroxybenzoate via the protocatechuate branch. Muconic acid yields ranged from about 14% using coniferyl alcohol as a substrate to about 93% utilizing benzoate. Compounds metabolized through vanillate (see FIG. 2; e.g. coniferyl alcohol, ferulate, and vanillin) demonstrated lower yields with accumulation of the intermediate vanillate. In contrast, compounds metabolized through the catechol branch (see FIG. 2; e.g. phenol, catechol, benzoate), as well as p-coumarate and 4-HBA, provided higher yields.

The performance of the engineered P. putida strain (KT2440-CJ103) was also studied in a fed-batch bioreactor experiment to understand the effects increased aeration, pH control, and a metered dosing of substrates on P. putida growth and the conversion of substrate to muconic acid. The results are illustrated in FIG. 5. Dissolved oxygen static (DO-stat) fed-batch biological conversion by KT2440-CJ103 yielded a muconic acid titer of about 13.5 g/L after about 78.5 hours using p-coumarate as a model lignin monomer substrate, over 15 times greater than the shake flask results shown in FIG. 4. Preliminary experiments indicated that muconic acid production from p-coumarate was significantly inhibited by the presence of excess glucose or acetate, potentially due to catabolite repression control or other regulatory inhibition. Therefore, DO-stat was used to maintain glucose levels below 1 mM while co-feeding p-coumarate and ammonium sulfate. During the course of cultivation, protocatechuate (protocatechuic acid) buildup occurred. Moreover, as the cultivation progressed past 60 hours, muconic acid concentrations plateaued and 4-HBA, a metabolite upstream of protocatechuic acid, accumulated.

FIG. 6 illustrates another set of fed-batch results where the P. putida KT2440-CJ103 strain was used to convert benzoate (benzoic acid) to muconic acid. In this example, glucose was used as a carbon source to support biological growth and function. Benzoate feeding was based on DO stat control, while pH was controlled using NaOH, resulting in sodium muconate as the predominant species at a pH of about 7. Muconate was produced at a titer of about 7.97 g/L after about 32 hours, while residual benzoic acid was present at about 0.94 g/L. After terminating the fed-batch run, cells were removed by centrifugation and filtration for subsequent broth processing.

Numerous other strains of P. putida KT2440 have been engineered and tested for their ability to convert lignin depolymerization products to muconic acid, several examples of which follow below.

### Example 1

Co-expression of decarboxylase subunits, EcdB and EcdD, to enhance the activity of the protocatechuate decarboxylase, AroY and, subsequently, increase production of muconic acid from aromatic molecules metabolized through protocatechuate.

As mentioned above, some experiments with P. putida KT2440-CJ103 for producing muconic acid from aromatic molecules metabolized through protocatechuate (PCA), including p-coumarate, 4-hydroxybenzoate (4-HBA), ferulate, and vanillin, demonstrated an accumulation of protocatechuate that reduced muconic acid yields. This suggested that the activity of the heterologously expressed protocatechuate decarboxylase that converts protocatechuate to catechol, AroY, may be insufficient. In an attempt to eliminate this bottleneck, enzymes from Enterobacter cloacae subsp. cloacae (ATCC 13047), EcdB and EcdD along with AroY (also from Enterobacter cloacae subsp. cloacae (ATCC 13047)) were engineered into a P. putida strain that was otherwise engineered to produce muconic acid from aromatic molecules. Metabolism of p-coumarate with the co-expression of AroY with EcdB (FIG. 7b) or with EcdB and EcdD (FIG. 7c), exhibited increased activity relative to AroY alone (FIG. 7a), which ultimately resulted in reduced accumulation of protocatechuate and greater production of muconic acid from p-coumarate.

Referring to FIG. 7a which illustrates the performance of P. putida strain KT2440-CJ102, the text in parenthesis, AcatRBCA::Ptac:catA ΔpcaHG::Ptac:aroY, means the following. First, the gene or genes immediately following a Δ symbol have been deleted from the genome. The double-colon following the deleted gene(s) refers to replacing the deleted gene(s) with the genetic element, gene or genes that immediately follow the double-colon. Finally, the single colon refers to genetic fusion of the gene before the colon to the gene following the colon. Thus, two genetic modifications were made to strain KT2440 to produce new strain KT2440-CJ102 (see FIG. 7a). First, the catRBCA genes were deleted and replaced by a DNA fragment comprising the Ptac promoter upstream of the catA gene and second, the pcaHG genes were deleted and replaced with a DNA fragment comprising the Ptac promoter upstream of the and aroY gene. Referring to FIG. 7b, the KT2440-CJ183 strain was constructed by modifying the KT2440 strain: first, the catRBCA genes were deleted and replaced by a DNA fragment comprising the Ptac promoter upstream of catA and second, the pcaHG genes were deleted and replaced with a DNA fragment comprising the Ptac promoter upstream of aroY and ecdB. Referring to FIG. 7c, the KT2440-CJ184 strain was constructed by modifying the KT2440 strain: first, the catRBCA genes were deleted and replaced with a DNA fragment comprising the Ptac promoter upstream of catA and second, the pcaHG genes were deleted and replaced with a DNA fragment comprising the Ptac promoter upstream of aroY, and ecdBD (ecdB and ecdD).

### Example 2

Over-expression of VanAB for enhanced conversion of vanillate (aromatic pathways) to protocatechuate and, subsequently, increased production of muconic acid from coniferyl alcohol pathway metabolites.

In addition to the AroY “bottleneck” described above, a considerable accumulation of vanillate was observed in the P. putida KT1440-CJ103 when metabolites from the coniferyl alcohol degradation pathway including coniferyl alcohol, ferulate, and vanillin were used as substrates for the production of muconic acid. This resulted in a reduction in the amount of muconic acid produced. FIGS. 8b-d illustrate the performance results of strains successfully engineered to remove this bottleneck, as demonstrated by reduced accumulation of vanillate and, thus, increased production of muconic acid relative to the empty vector control strain (FIG. 8a). A P. putida strain with increased expression of VanA is shown in FIG. 8b, a P. putida strain with increased expression of VanB is shown in FIG. 8c), and a third P. putida strain with increased expression of both VanA and VanB is shown in FIG. 8d. Unlike, the strains described above which involve genomic gene additions, VanA and VanB were added to the P. putida KT1440 strain by cloning the genes that encode these proteins into a broad-host range plasmid, pBTL-2, and transforming the resulting plasmids into P. putida KT2440-CJ102, where expression of these genes were driven constitutively by the Plac promoter in pBTL-2.

### Example 3

Deregulation of Carbon Catabolite Repression to enhance aromatic catabolism and, subsequently, increase production of muconic acid from aromatic molecules.

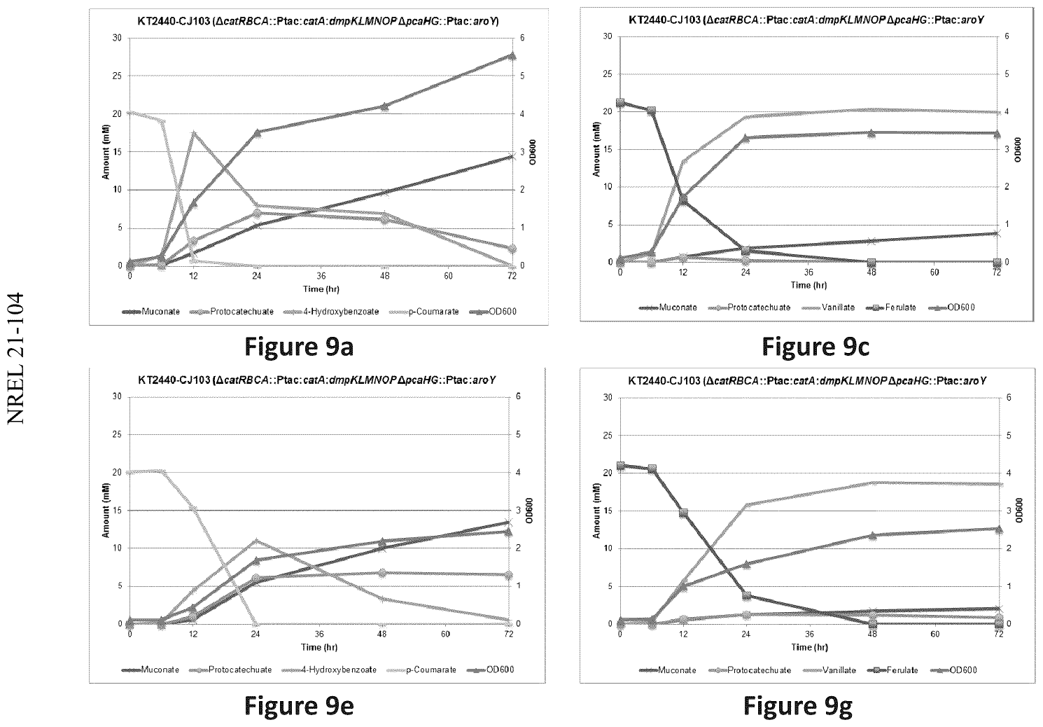

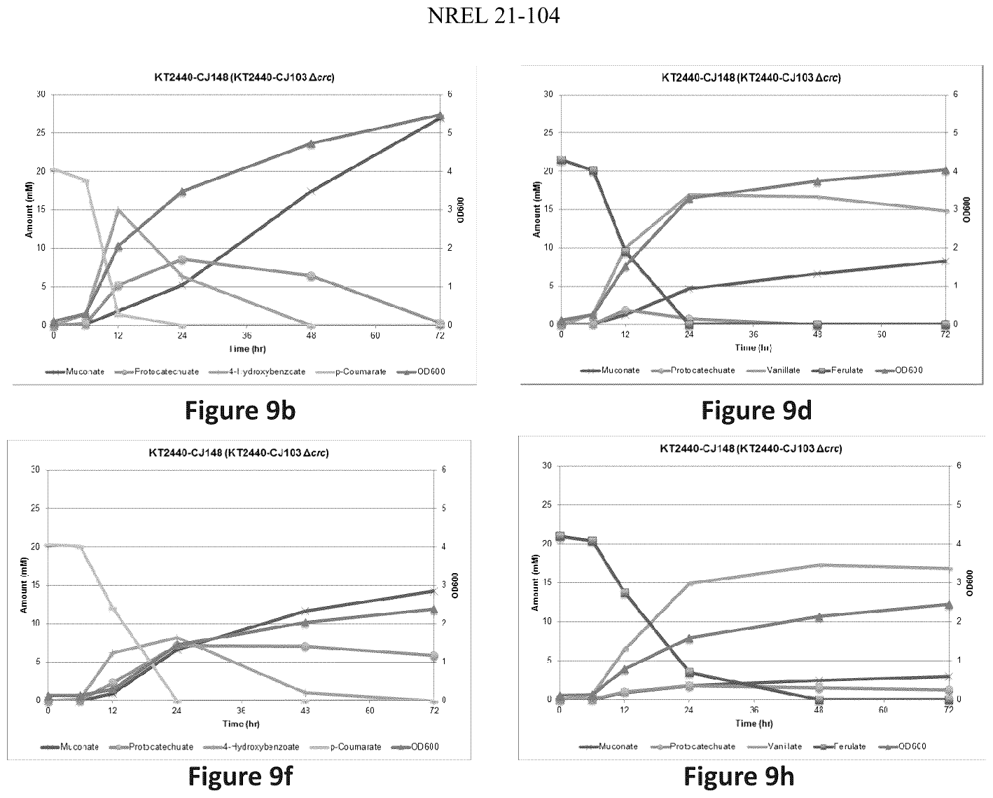

In Pseudomonads such as P. putida KT2440, the Catabolite Repression Control (Crc) protein binds targeted RNAs encoding proteins involved in catabolism and, thereby, may inhibit their translation and, thus, their activity. Pathways that enable catabolism of less preferred substrates are inhibited by Crc until preferred substrates, those which provide more carbon and/or energy, have been depleted. Among the targets of Crc regulation is catabolism of aromatic molecules. As shown in FIGS. 9a-h, deletion of Crc from P. putida KT2440-CJ102, resulted in less accumulation of intermediates from aromatic degradation, including 4-hydroxybenzoate and vanillate, which in turn resulted in higher production of muconic acid (see FIGS. 9b, 9d, 9f, and 9h), relative to the equivalent strain expressing Crc (see FIGS. 9a, 9c, 9e, and 9g). Thus, deletion of Crc represents a novel way to enhance the catabolism of aromatic molecules derived from lignin depolymerization and the subsequent production of molecules derived from them, such as muconic acid.

### Example 4

Expression of (−)-3-dehydroshikimate dehydratase, AsbF, and the protocatechuate decarboxylase, AroY, for production of muconic acid from sugars.

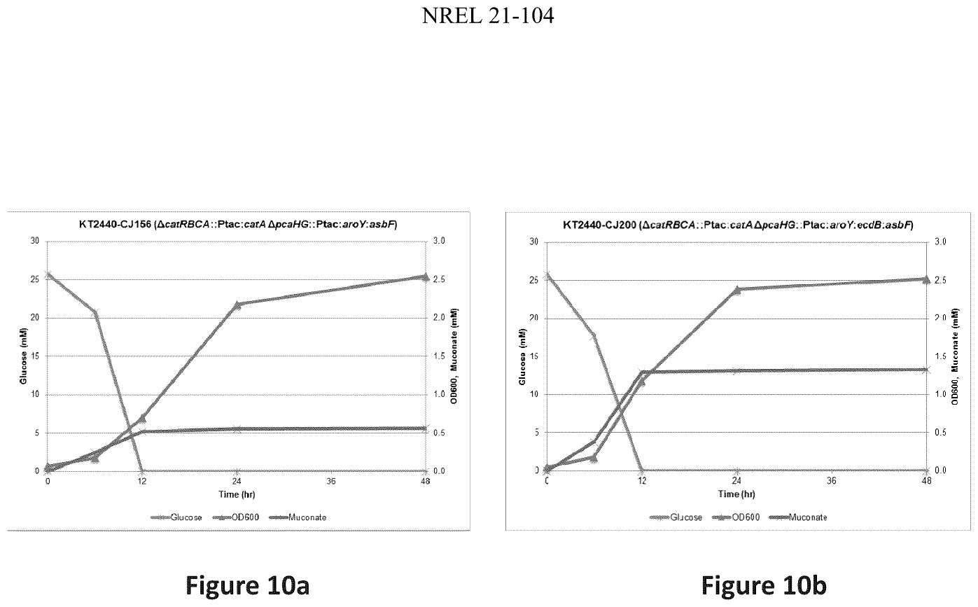

Heterologous expression of a (−)-3-dehydroshikimate (3-DHS) dehydratase, a protocatechuate decarboxylase, and a catechol dioxygenase may convert 3-DHS, an intermediate in the biosynthesis of aromatic amino acids, to protocatechuate, which may then be converted to catechol and cleaved to form muconic acid. FIGS. 10a-b demonstrate the production of muconic acid from glucose (FIG. 10a) by the engineered P. putida strain KT2440-CJ156 and KT2440-CJ200.

Referring to FIG. 10a, two genetic modifications were made to P. putida strain KT2440 to produce new P. putida strain KT2440-CJ156. First, the catRBCA genes were deleted and replaced by a DNA fragment comprising the Ptac promoter upstream of catA and second, the pcaHG genes were deleted and replaced with a DNA fragment comprising the Ptac promoter upstream of aroY from Enterobacter cloacae subsp. cloacae (ATCC 13047) and asbF from Bacillus cereus (ATCC 14579). Referring to FIG. 10b, two genetic modifications were made to P. putida strain KT2440 to produce new P. putida strain KT2440-CJ200. First, the catRBCA genes were deleted and replaced with a DNA fragment comprising the Ptac promoter upstream of catA and second, the pcaHG genes were deleted and replaced with a DNA fragment comprising the Ptac promoter upstream of aroY from Enterobacter cloacae subsp. cloacae (ATCC 13047), ecdB from Enterobacter cloacae subsp. cloacae (ATCC 13047), and asbF from Bacillus cereus (ATCC 14579). Such engineered P. putida KT2440 strains have several advantages over less stress-tolerant, plasmid-bearing hosts such as E. coli or Saccharomyces cerevisiae. In addition, expression of ecdB more than doubled the amount of muconic acid produced from glucose (see FIG. 10b), by increasing the activity of AroY. This pathway may be used to produce muconic acid from cellulose or hemicellulose-derived sugars such as glucose or xylose (or, for example, arabinose, mannose, galactose or rhamnose) as well as other bio-mass relevant feedstocks including, but not limited to acetate, and glycerol.

### Example 5

Strains, plasmid construction, and gene replacement methods. The example presented here illustrates the methods used to genetically modify P. putida KT2440 to construct the various modified P. putida strains described above.

Competent NEB (New England Biolabs, Inc., Ipswich, MA) C2925 and Life Technologies (Grand Island, NY) TOP10 was used for plasmid construction of cis,cis-muconate (muconic acid) producing and phenol utilizing strains, respectively. NEB 5-alpha F′I^{q} E. coli was used for all remaining plasmid constructions and were grown shaking at 225 rpm, 37° C., in LB Broth (Lennox) containing 10 g/L tryptone, 5 g/L yeast extract, 5 g/L NaCl or on LB plates containing 15 g/L agar, with either 10 μg/mL tetracycline or 50 μg/mL kanamycin. E. coli was transformed according to the manufacturer's instructions.

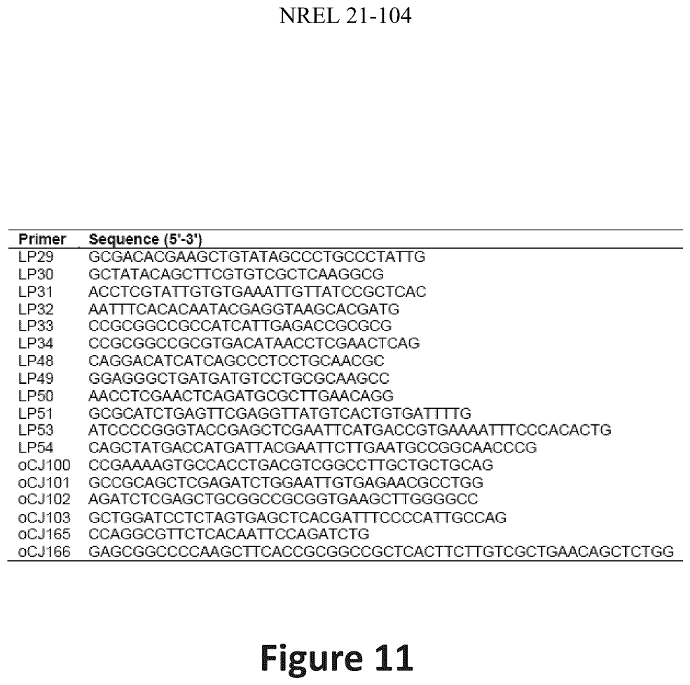

Q5® Hot Start High-Fidelity 2× Master Mix (NEB) and primers synthesized by IDT (Integrated DNA Technologies, Inc., Iowa) were used in all PCR amplification for plasmid construction. Primer sequences are shown in FIG. 11. Plasmids were assembled using Gibson Assembly® Master Mix (NEB) according to the manufacturer's instructions. The sequences of all plasmid inserts were confirmed using Sanger sequencing

Plasmids for gene replacement were constructed in pCM433 (Addgene Inc., Cambridge, MA)1 or pK18mobsacB from ATCC (American Type Culture Collection, Manassas, VA), both of which are unable to replicate in P. putida and contain antibiotic resistance genes to select for integration of the plasmid into the genome by homologous recombination and sacB to counterselect for recombination of the plasmid out of the genome. Plasmids for expression of vanA and/or vanB were constructed in pBTL-2 (Addgene Inc., Cambridge, MA), which is able to replicate in P. putida.

The pCM433-based integration vector used to replace catRBCA with Ptac:catA (pMFL22) was constructed by Gibson assembly of three PCR products: LP29 and LP33 were used to amplify the targeting region upstream from catA, LP30 and LP31 were used to amplify the Ptac promoter from Sigma pFLAG-CTC, LP32 and LP34 were used to amplify the entire coding region of catA including its native RBS. After assembly, the 2.2 kb fragment was amplified by PCR using primers LP29 and LP34, and cloned into the pCM433 vector using NotI sites.

The pK18mobsacB-based plasmid for integration of the phenol monooxygenase genes (pMFL56) was constructed by Gibson assembly of three PCR fragments using primers LP53 and LP48 to amplify the catA targeting region, LP49 and LP50 for amplification of six phenol monooxygenase genes, dmpKLMNOP using pVI1261 as the template (provided by Dr. Victoria Shingler from the Department of Molecular Biology at Umeå University), and primers LP51 and LP54 for amplification of the targeting region downstream from catA. Fragments were then cloned into pK18-mob vector using NotI sites.

In the plasmid for replacement of pcaHG with Ptac:aroY (pCJ023), the aroY gene (ADF69416) from Enterobacter cloacae ATCC13047 was optimized for expression in P. putida KT2440 using DNA 2.0's Gene Designer software and synthesized in two overlapping DNA fragments by IDT. The first fragment also contained the Ptac promoter, which was separated from the initiating ATG by a ribosome binding site with the sequence AGAGGAGGGAGA. These fragments were then assembled by Gibson assembly and Ptac:aroY was amplified from this assembly with primers oCJ165 and oCJ166. Approximately 1 kb regions upstream and downstream of pcaHG were amplified using oCJ100/oCJ101, and oCJ102/oCJ103, respectively. The upstream targeting region, Ptac:aroY, and the downstream targeting region were then assembled into pCM433 linearized with restriction enzymes AatII and SacI (NEB).

Gene replacement plasmids were transformed into P. putida strains by electroporation. LB broth was inoculated to an OD600 of about 0.02 and incubated shaking at 225 rpm, 30° C., until an OD600 of 0.5-0.7 was reached. Cells were then centrifuged at 4° C., washed twice in ice-cold water and once in ice-cold 10% glycerol or 3 mM potassium phosphate (KPi), pH 7.0, before being resuspended in 1/100 of the culture's original volume of 10% glycerol (or 3 mM KPi). Cells were then stored at −80° C. or transformed by electroporation immediately. For transformation, 5 μL (200 ng-2 μg) of plasmid DNA was added to 50 μL of the electrocompetent cells, transferred to a chilled 0.1-cm electroporation cuvette, and electroporated at 1.6 kV, 25 uF, 200 ohms. 450 μL SOC outgrowth medium (NEB) was added to the cells immediately after electroporation and the resuspended cells were incubated shaking at 225 rpm, 30° C., for one hour. The entire transformation was plated on LB agar plates containing appropriate antibiotics (30 μg/mL tetracycline for pCM433-based plasmids, 50 μg/mL kanamycin for pK18mobsacB-based plasmids) and incubated at 30° C. overnight. Transformants were restreaked for single colonies on LB agar and incubated at 30° C. overnight to reduce the possibility of untransformed cells being transferred. For sucrose counter-selection, restreaked transformants were streaked for single colonies on YT+20 or 25% sucrose plates (10 g/L yeast extract, 20 g/L tryptone, 250 g/L sucrose, 18 g/L agar) and incubated at 30° C. overnight. P. putida KT2440 containing the sacB gene can grow, although very slowly, on YT+20% or 25% sucrose media. Therefore, colonies presumed to have recombined the sacB gene out of the genome—those colonies that were larger than most—were restreaked on YT+25% sucrose plates and incubated at 30° C. overnight to reduce the possibility that cells that had not recombined would be carried along with sucrose resistant isolates. Colonies from the second YT+25% sucrose plates were subjected to colony PCR to check for gene replacement at both junctions. These isolates were also plated on LB plates containing appropriate antibiotics to ensure that they had lost antibiotic resistance and, thus, represented pure gene replacements.

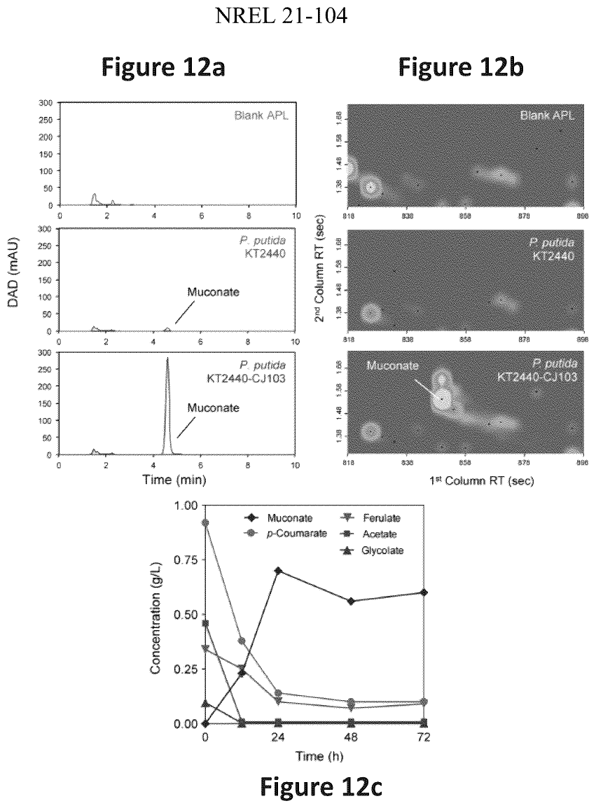

Referring to FIGS. 12a-c, these figures provide experimental data obtained from “proof of concept” tests to show the viability of utilizing engineered microorganisms to metabolize lignin-derived raw materials to produce muconic acid. In this example, alkaline pretreatment with NaOH and anthraquinone (AQ) was applied to corn stover at 70 mg NaOH/g dry biomass with an AQ concentration of 0.2 wt % of dry stover. The resulting alkaline pretreated liquor (APL) stream contains a substantial amount of lignin-derived aromatics such as p-coumarate and ferulate as well as acetate, biomass extractives, and very minor concentrations of sugars (<0.5 g/L of any monomeric sugar). The pH of APL was reduced to about 7 with the addition of H₂SO₄. The liquor was then filtered through a 0.2 μm filter for sterilization and to remove residual solids.

Flasks containing 25 mL of M9 minimal medium supplemented with 0.9×APL were inoculated with P. putida KT2440 or KT2440-CJ103 and cultured for three days. Following biological conversion, cells were removed by centrifugation and activated carbon (12.5 wt/vol %) was added to the remaining culture media to remove non-target aromatics and facilitate analysis by HPLC. Analysis by HPLC detected significant levels of muconic acid in cultures grown with P. putida KT2440-CJ103, while no significant quantities were detected in the blank APL control sample or with the native P. putida KT2440 (see FIG. 12a). Likewise, analysis of derivatized acids in unpurified culture samples by GCxGC-TOFMS (time-of-flight mass spectrometry) confirmed the identity of muconic acid and displayed comparable trends in concentration, as shown in of FIG. 12b.

To track the conversion of primary aromatic and nonaromatic components in APL during shake flask cultivation, GCxGC-TOFMS was also employed. Analysis of APL determined that p-coumarate and ferulate were initially present at significant levels (0.92 g/L and 0.34 g/L, respectively), in addition to the short chain acids glycolate and acetate (0.46 g/L and 0.10 g/L, respectively), as shown in FIG. 12c. Other aromatic acids, including benzoate, caffeate, vanillate, and 4-hydroxybenzoate, were not detected in significant levels (>0.01 g/L). During shake flask cultivations, P. putida KT2440-CJ103 rapidly consumed glycolate and acetate, which can be used as sources of carbon and energy for growth. The primary aromatic components, p-coumarate and ferulate, were converted to 0.70 g/L of muconate after 24 hours (see FIG. 12c). Based on the consumption of these two major aromatic acids, the molar yield to muconate was 67%.

### Example 5: Strains, Media, and Growth Conditions

P. putida KT2440 (ATCC 47054) and its derivatives were grown shaking at 225 rpm, 30° C., in LB Broth or LB plates. During gene replacement, sucrose selection was performed on YT+25% sucrose plates (10 g/L yeast extract, 20 g/L tryptone, 250 g/L sucrose, 18 g/L agar). Shake flask and bioreactor experiments were performed using modified M9 minimal media containing 13.56 g/L disodium phosphate, 6 g/L monopotassium phosphate, 1 g/L NaCl, 2 g/L NH4Cl, 2 mM MgSO4, 100 mM CaCl₂), and 18 mM FeSO4.

### Example 6: Shake Flask Experiments with Model Monomers and APL

Fed batch and shake flask experiments were performed using 125 mL baffled flasks containing 25 mL modified M9 media supplemented with 10 mM sodium benzoate, coniferyl alcohol, ferulate, vanillin, caffeate, p-coumarate, 4-hydroxybenzoate or 5 mM phenol and 20 mM sodium acetate or 10 mM glucose. For shake flask experiments in which cells were grown on alkaline pretreated liquor (APL), modified M9 medium was supplemented with APL at a concentration of 0.9×. Cultures were inoculated with cells washed in modified M9 medium to OD₆₀₀ 0.05, then incubated shaking at 30° C., 225 rpm. Every 12 hours cultures were sampled for HPLC, OD₆₀₀, and pH measurement. For cultures at pH>7.4 or <6.6, the pH was adjusted to 7.0 by adding 1N HCl or 1N NaOH. 20 mM sodium acetate or 10 mM glucose was added before returning the cultures to the incubator.

### Example 7: Fed-Batch Cultivation

A seed batch culture of P. putida KT2440-CJ103 was started in a shake flask and grown overnight in LB, 30° C., 225 rpm. The next morning, cells were centrifuged 3800×g, 10 minutes and washed once with modified M9 medium containing 10 mM glucose. Cultures were transferred to 700 mLs of the same medium in a 2 L Applikon (Applikon Biotechnology, Inc.) EZ Control 2 L bioreactor, starting at an initial OD₆₀₀ of 0.2. Base pH was controlled by 2N NaOH to pH 7. The temperature was maintained at 30° C. Mixed air was used to deliver oxygen at a flow rate of 2 L/min. DO saturation was manually adjusted to ˜50% by varying stirrer speed, from 250 to 650 rpm, and then maintained at 650 rpm for the duration of the experiment. At 5 hours, 2 mM p-coumarate was added. When glucose was consumed at ˜11.5 h, a large spike in DO was observed, indicating that glucose was depleted and confirmed by YSI analysis. A separate pump was computer programmed to deliver for 30 seconds (˜2.4 mL) a p-coumarate:glucose:ammonium sulfate (68.4:36.5:9 g/L) feed when DOT (dissolved oxygen tension) levels reached ≥75%. The feed caused a temporary drop in DOT to ˜50%, until glucose concentrations fell again. As expected, DOT oscillations proceeded at similar frequencies, until the p-coumarate:glucose:ammonium sulfate feed was terminated at 75.5 hours and the bioreactor was shut down at 78.5 hours.

### Muconic Acid Separation, Purification, and Upgrading

Referring again to FIGS. 1a-c, microbial catalysis operations 140 are only one part of a fully-integrated biorefinery 100. Once a production-scale culture has successfully produced muconic acid, several processing steps remain to obtain a purified muconic acid stream and/or final product(s) that may be manufactured from muconic acid; e.g. adipic acid, nylon-6,6. As discussed previously, one or more separation operations 150 may be required to generate a purified and usable muconic acid stream.

Separation/purification operations 140 are needed to generate a usable muconic acid stream for a number of reasons. For example, a wide variety of impurities may be introduced during the biological production of muconic acid, similar to the challenges faced with other target bio-derived molecules (e.g., ethanol, succinic acid, lactic acid). These impurities may include fermentation salts, nutrients and media to support growth, unconverted substrate, extracellular proteins and lysed cell contents, as well as the buildup of non-target metabolites. Accumulation of these constituents in culture broth may vary greatly depending on the microorganism, substrate used for conversion, biological growth conditions and bioreactor design, and broth pretreatment. Likewise, utilization of monomer streams derived from complex lignocellulosic biomass may vary greatly depending on the biomass fraction of interest (e.g., cellulose, hemicellulose, lignin), choice of feedstock (e.g., herbaceous, hardwoods, softwoods), and depolymerization technology.

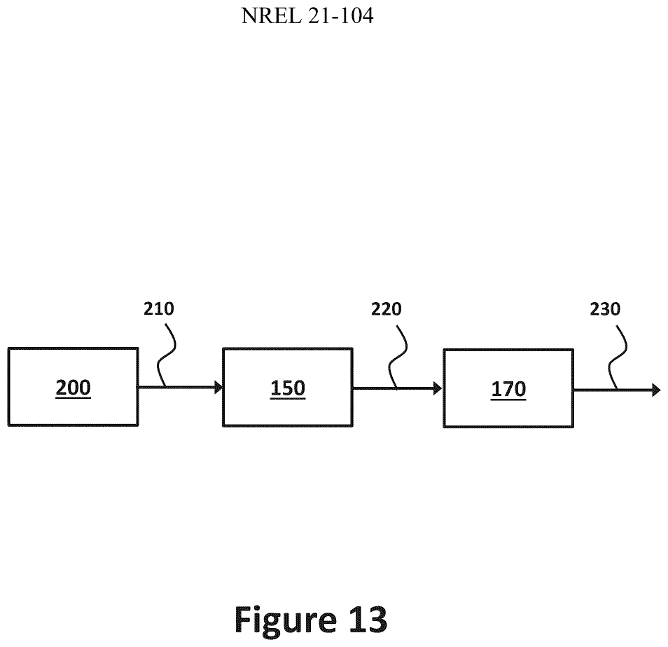

Therefore, a culture broth containing a target muconic acid titer will need to be processed before any final products may be manufactured. So, referring to FIG. 13, a muconic-acid containing culture broth 210 may be fed from a bioreactor 200 to a separation/purification operation 150 to produce a purified muconic acid stream of sufficient quality that it may then subsequently be fed to an upgrading 170 section of the biorefinery. Upgrading may include, for example, catalytic hydrogenation of the purified muconic acid to produce adipic acid. The adipic acid may then be further reacted to produce nylon-6,6. In some embodiments of the present invention, the process for producing muconic acid from biomass may further comprise at least one initial separation step that produces the at least one biomass feed stream, wherein the separation step comprises receiving a biomass raw material comprising at least two substituents comprising at least two of lignin, cellulose, hemicellulose, or mixtures thereof, and separating at least two of the substituents to produce the at least one biomass feed stream. In some embodiments of the present invention, the separation separates lignin from polysaccharides to produce a first biomass feed stream comprising lignin, and a second biomass feed stream comprising polysaccharides. The polysaccharide stream may comprise at least one of cellulose, hemicellulose, and mixtures thereof.

Some exemplary technologies that may be utilized in the separation/purification operation 150 include at least one of affinity chromatography, ion exchange chromatography, solvent extraction, liquid-liquid extraction, distillation, filtration, centrifugation, electrophoresis, hydrophobic interaction chromatography, gel filtration chromatography, reverse phase chromatography, chromatofocusing, differential solubilization, preparative disc-gel electrophoresis, isoelectric focusing, HPLC, and/or reversed-phase HPLC.

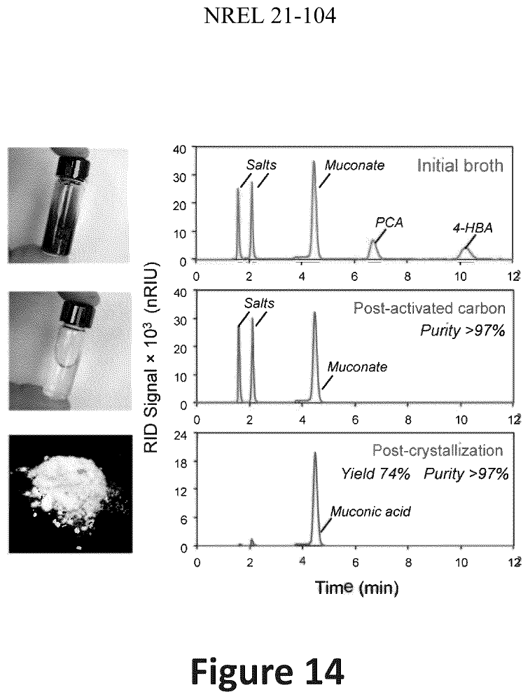

Cell removal from the broth may be achieved by a variety of solid removal unit operations. Some examples include filtration, centrifugation, and combinations thereof. Once the microorganism cell matter has been removed, further impurity removal operations may be utilized. For example, the biological ring opening of muconate allows for facile purification from culture media containing non-target aromatic metabolites (e.g., unreacted protocatechuic acid and 4-hydroxybenzoic acid) using activated carbon due to the high adsorption affinity of oxygenated aromatics in comparison to aliphatic acids. FIG. 14 illustrates a comparison of an initial broth (from FIG. 5) and the same broth after treatment with activated carbon. In this example, activated carbon was added to the culture media at about 12.5% (wt/vol) and the resultant mixture was stirred for about 1 hour. This treatment step resulted in nearly complete removal (below detectable limit by HPLC) of protocatechuic acid and 4-hydroxybenzoic acid, while the majority of the muconic acid (89% of initial culture media concentration, mass/vol) remained in solution.

The activated-carbon-treated muconic acid was then crystallized by reducing the pH and temperature, which is enabled due to the strong pH and temperature dependence of dicarboxylic acids. At a pH of about 2 and temperature of about 5° C., muconic acid readily precipitated from solution and the muconic acid crystals were recovered by vacuum filtration. This method recovered 74% of the muconic acid initially present in the activated-carbon-treated muconic acid stream, with a high degree of purity (>97%), as shown in the bottom plot of FIG. 14.

The activated carbon treatment of cell-free, muconic acid containing broth was tested a second time, this time on the broth resulting from culture shown in FIG. 6. The separation process initially consisted of activated carbon purification to remove soluble organic broth impurities and pH/temperature shift crystallization to precipitate muconic acid from purified broth solution and recover solid crystals for drying, similar to what was described above. However, in this example, an additional step, ethanol dissolution and microfiltration was utilized to remove bulk inorganic salts. Purity results for separation tests completed on muconic acid generated by biocatalysis compared to commercial grade muconic acid are summarized in the table below.

TABLE 1
Purity of commercial muconic acid (chemical origin) and biologically
derived muconic acid after sequential treatment.

| Sample | AC | pH 2 | EtOH | |
|---|---|---|---|---|
| Origin | Treated | Crystallized | Dissolved | Purity (%) |
| Commercial | N | N | N | 97.83 ± 0.07% |
| Chemical | | | | |
| Biological | Y | Y | N | 97.86 ± 0.05% |
| Conversion | | | | |
| Biological | Y | Y | Y | 99.76 ± 0.04% |
| Conversion | | | | |

^{a}Standard deviation values reported for triplicate sample measurements.

An activated carbon loading of about 2 wt/vol % was needed to remove residual benzoate from this culture broth to below detectable limits (as determined by high performance liquid chromatography diode array detection. Color compounds in the broth were also removed to a significant extent, turning the broth from a coffee-colored appearance to semi-clear; however, non-selective adsorption of resulted in a 16% reduction in muconic acid broth concentration (6.86 g/L).

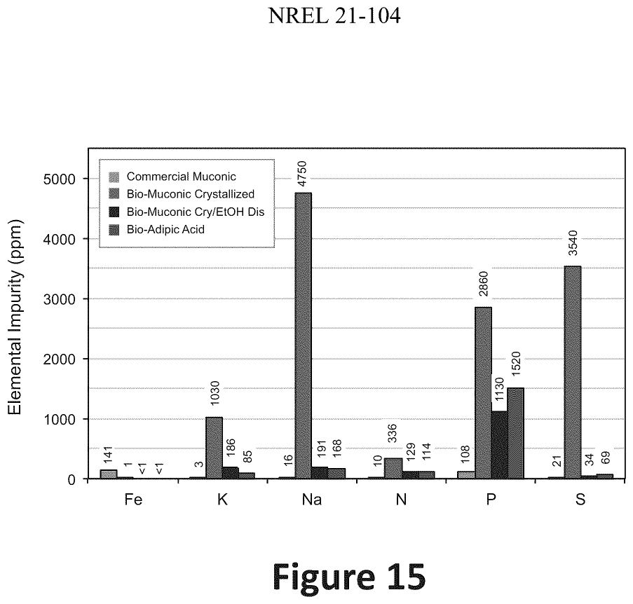

Following the activated carbon treatment, muconic acid was precipitated from the broth by pH/temperature shift crystallization. By adjusting the broth pH to 2 with sulfuric acid and reducing the temperature to 5° C., muconic acid readily precipitated. Precipitated muconic acid crystals were then vacuum filtered (0.2-μm PES) and dried in a vacuum oven for about 48 hours. Purity analysis by differential scanning calorimetry (DSC) melting point analysis determined the muconic acid crystals were about 97.83±0.05% pure at this stage. Combustion analysis of muconic acid crystals at 700° C. measured a sample ash content of 1.44% (wt/wt), and elemental analysis by ICP-MS and nitrogen chemiluminescence identified major impurities as sodium (4750 ppm), sulfur (3540 ppm), phosphorus (2860 ppm), potassium (1030 ppm), and nitrogen (336 ppm), as shown in FIG. 15. As shown in FIG. 15, the elemental impurities identified in biologically derived muconic acid differed significantly compared to the impurities observed in chemically derived muconic acid obtained from Sigma Aldrich, with the latter being much lower in sodium, sulfur, phosphorous, potassium, and nitrogen, but higher in iron and chloride. The major inorganic impurities identified in the bio-derived muconic acid are known poisons to platinum group metals, requiring removal strategies prior to catalysis. Likewise, polymer-grade adipic acid requires trace levels of iron (<0.2 ppm) and nitrogen (<20 ppm N), necessitating further treatment.

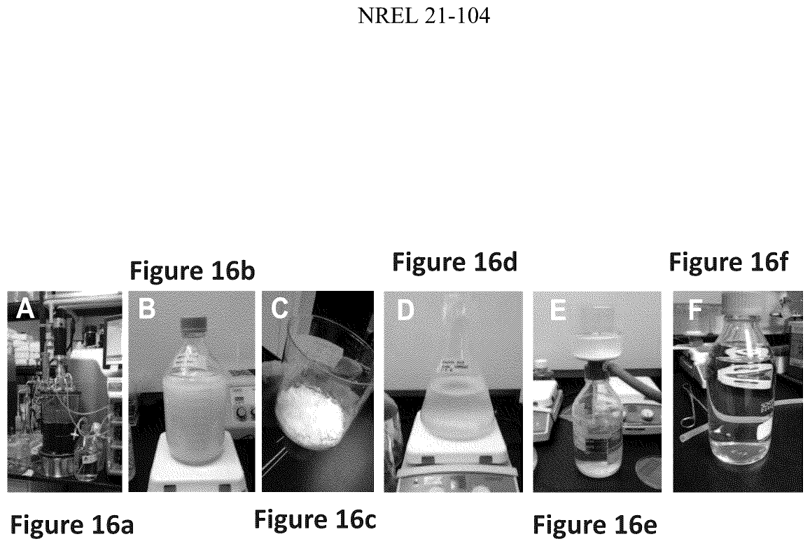

In order to reduce the level of inorganic impurities in the bio-derived muconic acid and generate a feed stream with sufficient purity and quality to enable successful downstream upgrading, the muconic acid crystals produced in the previous steps were dissolved in ethanol and filtered through a 0.2-μm PES membrane. Upon ethanol dissolution, the muconic acid-ethanol solution was initially cloudy due to insoluble salts, whereas after filtration the solution was very clear (see FIGS. 16a-f). The initial muconic acid culture broth appeared dark coffee colored (FIG. 16a). Activated carbon treatment of the broth significantly removed color compounds, while adjustment to a pH of about 2 initiated crystal formation (FIG. 16b). Filtration and drying of the purified broth produced a white crystal solid, with a purity of 97.86±0.05% by DSC melting point analysis (FIG. 16c). Muconic acid crystals dissolved in ethanol resulted in a cloudy solution (FIG. 16d), that upon 0.2-μm microfiltration (FIG. 16e) resulted in a clear solution (FIG. 16f) with a final muconic acid purity of 99.76±0.04% upon drying. Analysis of filtered and dried muconic acid after ethanol dissolution revealed an overall DSC-purity of 99.76±0.04% as shown in Table 1 above, with a significant reduction in elemental impurities.

Analysis by ICP-MS determined that sodium was reduced by 96%, sulfur by 99%, phosphorous by 60%, potassium by 82%, and nitrogen by 62% (see FIG. 14), consistent with reductions in low-concentration elements (<100 ppm aluminum, chlorine, magnesium) (results not shown). Biocatalysis-derived muconic acid iron levels were much lower (<1 ppm) compared to muconic acid of chemical origin (141 ppm). Nitrogen levels in bio-muconic acid were still above the polymer precursor specification of 20 ppm, due to residual fermentation proteins that were not removed during activated carbon treatment and microfiltration.

Referring again to FIG. 13, the details provided above demonstrate that biomass-derived muconic acid can be successfully produced by genetically engineered microorganism modified to funnel both lignin depolymerization products and polysaccharide depolymerization products to muconic acid with high yield and selectivity in a bioreactor 200. The muconic acid containing broth 210 can then be successfully treated in a separation/purification operation 150 to produce a purified muconic acid stream 220. This purified muconic acid stream 220 can then be used in an upgrading operation 170 to produce value-added final products, including fuels and commodity chemicals.

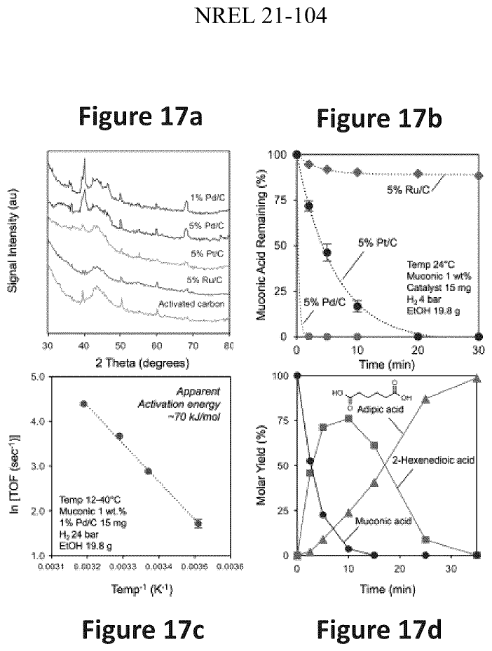

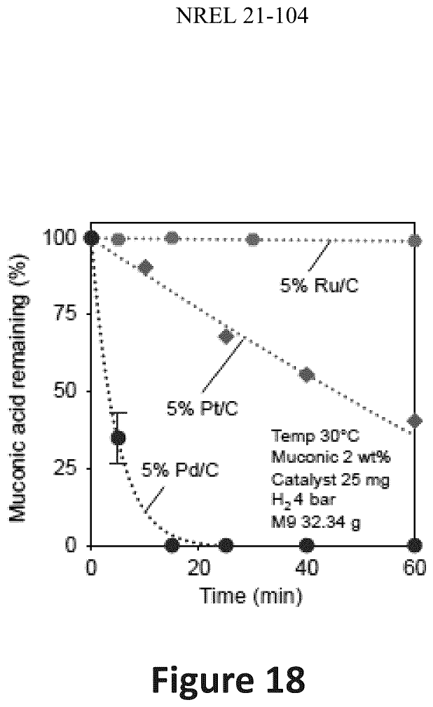

As described above, a high volume intermediate that may made from muconic acid is adipic acid, which may then be further converted to nylon-6,6. To evaluate the feasibility of converting bioderived muconic acid to adipic acid, catalyst screening experiments were conducted to identify highly active materials for muconic acid hydrogenation at low temperature and pressure. Commercial noble metal catalysts supported on carbon were initially tested at 5 wt % loading, including palladium, platinum, and ruthenium. Characterization of the virgin catalyst materials (see FIG. 17a and FIG. 18) revealed the metals were dispersed as small crystallites, with comparable support surface areas (705-1075 m²/g), pore volumes (0.51-0.71 mL/g), and a wider range of exposed active metal areas (22-51% dispersion). Screening experiments found that Pd/C was by the most active catalyst, with consistent activity trends when using M9 culture media (aqueous solution containing salts to support biological growth) or ethanol, as a representative protic polar organic solvent. During the course of the reaction, 2-hexenedioic acid was observed as the primary intermediate, likely due to the low temperature conditions that minimized competing nonselective reaction pathways. For reactions that went to completion with Pd, selectivity to adipic acid was >97% (mol/mol) (See FIG. 17b).

Additional hydrogenation conditions were examined with Pd/C to (i) determine its activity under surface reaction controlling conditions, (ii) evaluate the apparent activation energy for muconic acid reduction, and (iii) demonstrate its utility with muconic acid recovered from fed-batch biological conversion. Experiments conducted at two different Pd loadings (1 wt % and 2 wt % Pd/C) exhibited comparable turn over frequencies (TOF; 23±6/s and 30±6/s, respectively, at 24 bar of hydrogen and 24° C. in ethanol, 15 mg catalyst, stirring at 1600 rpm), supportive of surface reaction controlling conditions. Experiments to measure the hydrogenation rate of muconic acid at varying temperatures estimated an apparent activation energy of ˜70 kJ/mol (see FIG. 17c), significantly above values indicative of mass transfer limitation (<20 kJ/mol). Hydrogenation with Pd/C was then demonstrated with muconic acid obtained from fed-batch biological conversion of p-coumarate after activated carbon purification and crystallization. Hydrogenation at room temperature progressed rapidly in a series reaction (see FIG. 17d, muconic acid TOF 25±3 sec⁻¹), resulting in high purity adipic acid as the final product (>97% mass/mass). After the reaction, analysis of the ethanol solvent indicated that leaching of Pd occurred to a minor extent (7 mg/L, 0.8% of the loaded metal), which may occur due to the acidic liquid phase conditions employed.

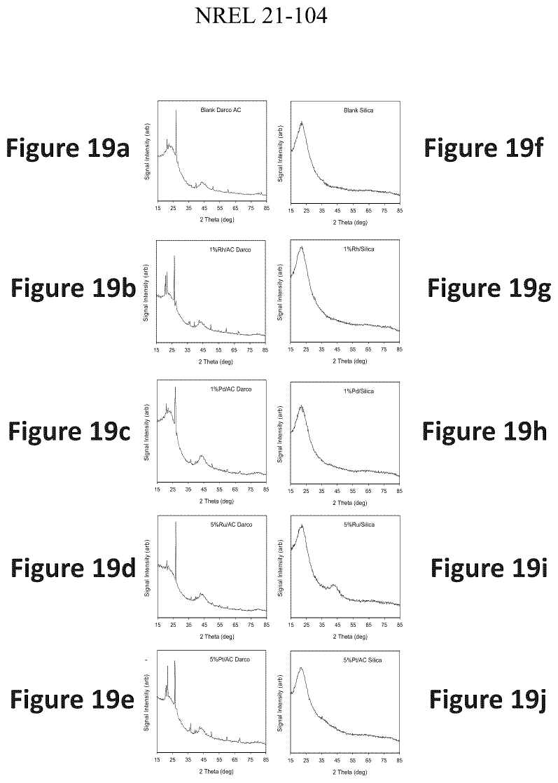

Additional studies were completed to evaluate the hydrogenation of bioderived muconic acid to adipic acid. Batch reactor catalyst screening experiments were conducted with platinum group metals to evaluate their activity and stability against leaching during muconic acid hydrogenation. Catalysts were synthesized using powdered Darco activated carbon (AC) and Davisil silica supports sieved to >270 mesh (<53 m) to minimize the impact of mass transfer during batch conditions. Metals precursors were loaded onto their respective supports, and catalysts were reduced in hydrogen prior to characterization to determine their metal loading and dispersion, support surface area, pore volume and pore diameter, and x-ray diffraction (XRD) spectra, as shown in Table 2 and FIGS. 19a-j.

TABLE 2
Properties of virgin activated carbon (AC) and
silica powdered catalysts used in batch screening
reactions for muconic acid hydrogenation.

| Catalyst | S_{BET} | Pore vol. ^{a} | Pore dia. ^{a} | Dispersion ^{b} |
|---|---|---|---|---|
| (nominal) | (m² g⁻¹) | (cm³ g⁻¹) | (Å) | (%) |
| 1% Pd/AC | 768 | 0.514 | 9.71 | 13 |
| 1% Rh/AC | 971 | 0.708 | 9.83 | 69 |
| 5% Ru/AC | 590 | 0.588 | 9.69 | 10 |
| 5% Pt/AC | 882 | 0.657 | 9.71 | 60 |
| 1% Pd/SiO₂ | 466 | 0.774 | 9.74 | 28 |
| 1% Rh/SiO₂ | 480 | 0.804 | 9.81 | 62 |
| 5% Ru/SiO₂ | 428 | 0.686 | 9.75 | 17 |
| 5% Pt/SiO₂ | 454 | 0.811 | 9.79 | 47 |

High surface areas were observed for both AC (590-971 m² g⁻¹) and silica (428-480 m²/g) supported catalysts, with higher metal loading materials generally showing lower surface areas. Support pore volumes (AC 0.514-0.708 cm³/g, silica 0.686-0.811 cm³/g) and pore diameters (AC 9.69-9.83 Å, silica 9.74-9.81 Å) were also comparable. Elemental analysis determined metal loadings were near their nominal values and XRD analysis confirmed the absence of sharp prominent peaks due to large metal crystallites. Chemisorption analysis measured dispersions were within the range of 10-62%, likely due to differing metal precursor and support material interactions during synthesis. Due to varying active metal crystallite surface areas, observed catalyst activities for muconic acid hydrogenation were normalized to dispersion values to allow for turn-over-frequency (TOF) comparisons between metals (e.g., moles of compound reacted per second, divided by the moles of surface metal atoms measured by dispersion).

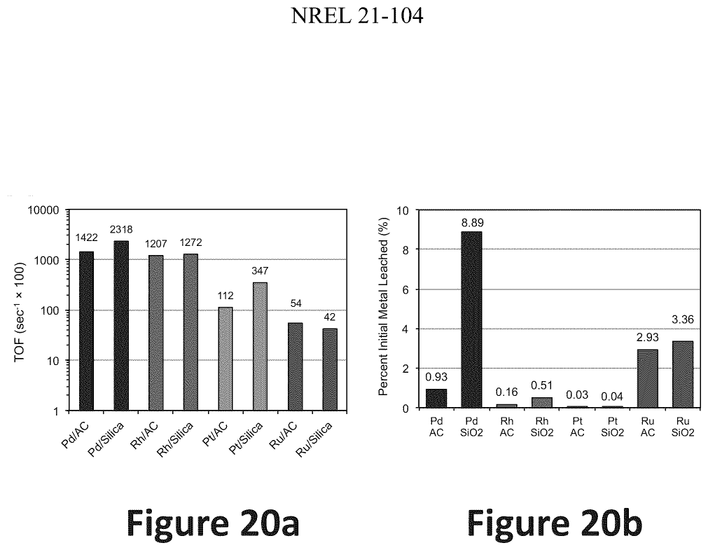

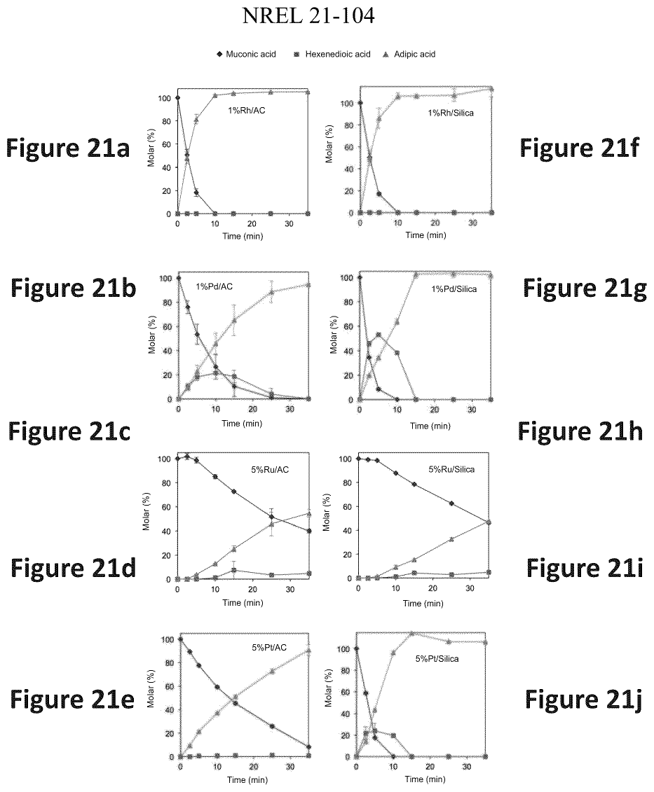

As shown in FIG. 20a, pronounced differences were observed in the muconic acid hydrogenation activity between the platinum group metals tested during batch reactor screening experiments (see FIG. 21a-h for conversion profiles). Pd and Rh displayed highest TOF for muconic acid hydrogenation on both AC and silica supports, with Pd ranging from 14-23 sec⁻¹ and Rh ranging from 7-8 sec⁻¹. In comparison, Pt displayed a TOF ranging from 1-3 sec⁻¹, while Ru ranged from 0.4-0.5 sec⁻¹. For batch reactions tested at room temperature, 2-hexenedioic acid was the primary intermediate observed, with trace levels of 3-hexenedioic acid identified on occasion (Scheme 1). For all catalysts screened in the batch system, mass balance closure was typically within +/−10%, with adipic acid as the only product observed upon completion of the reaction.

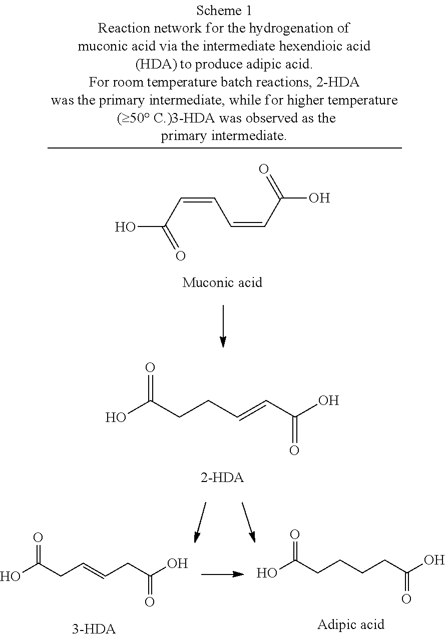 [SMILES: O=C(O)/C=C\C=C/C(=O)O ; O=C(O)/C=C/CCC(=O)O ; O=C(O)C/C=C/CC(=O)O ; O=C(O)CCCCC(=O)O]

In addition to differing hydrogenation activity, catalyst metal leaching also varied significantly based on both the metal and choice of support, as shown in FIG. 20b. Despite exposure to reaction conditions for only 35 min, 1% Pd/SiO₂ displayed the greatest amount of leaching on a percent of initial metal loading basis (8.89%), while 1% Pd/AC leached to a lesser extent (0.93%). Leaching was also observed for 5% Ru/AC (2.93%) and 5% Ru/SiO2 (3.36%) with muconic acid. In contrast, metal leaching for 1% Rh/AC and 1% Rh/SiO₂ was at trace levels, at 0.51% and 0.16%, respectively, while leaching was negligible for both 5% Pt/SiO2 (0.04%) and 5% Pt/AC (0.03%). Based on the high activity for muconic acid hydrogenation and stability against leaching, Rh/AC was chosen for further time-on-stream testing in a continuous trickle-bed reactor.

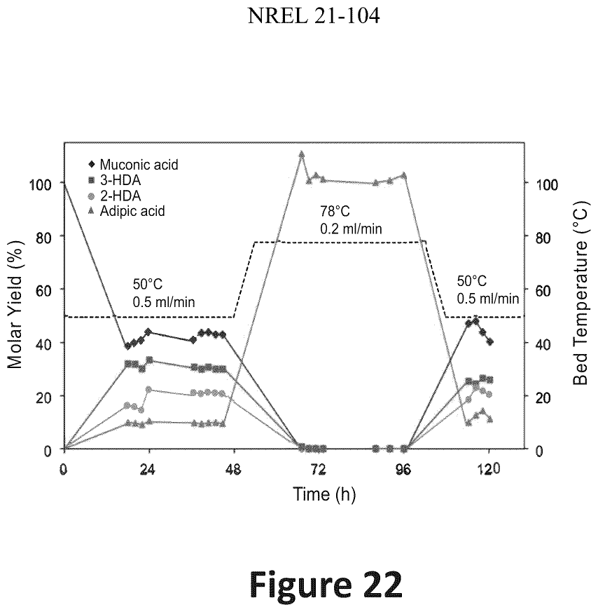

Based on the activity and stability of Rh during batch reactions, continuous trickle bed reactor studies were conducted to determine its 100-h time-on-stream stability, as shown in FIG. 22. Initially, a 1% Rh/AC catalyst was prepared on Norit AC granules crushed and sieved between 100-80 mesh (150-180 μm) to minimize the catalyst bed pressure drop. The catalyst was characterized to determine its virgin and post-reaction properties, as shown in Table 3. Characterization of the virgin 1% Rh/AC catalyst showed high surface area (1029 m² g⁻¹), with comparable pore volume (0.455 cm³ g⁻¹) and average pore diameter (9.69 Å) to the powder Rh/AC catalyst. XRD analysis confirmed the absence of sharp, prominent peaks due to large metal crystallites. Distinct differences were observed in XRD spectra of the powder and granule AC supports, likely due to different vendor carbon sources and/or activation techniques. The metal crystallite dispersion of the granule 1% Rh/AC catalyst was also lower (11.2%) compared to the powder catalyst.

TABLE 3
Properties of virgin and post-reaction 1% Rh/AC
granule catalyst used in the 100-h time-on-stream
stability test for muconic acid hydrogenation.

| Catalyst | ICP | S_{BET} | Pore vol. ^{a} | Pore dia. ^{a} | Dispersion ^{b} |
|---|---|---|---|---|---|
| (nominal) | (%) | (m² g⁻¹) | (cm³ g⁻¹) | (Å) | (%) |
| Virgin | 0.8 | 1029 | 0.46 | 9.69 | 14% |
| 1% Rh/AC | | | | | |
| Post Reaction | 0.9 | 1130 | 0.52 | 11.43 | 21% |
| 1% Rh/AC | | | | | |

^{a} Pore volume and pore diameter (average) determined by BJH desorption.
^{b} Dispersion calculated based on chemisorption and ICP measured metal loading.

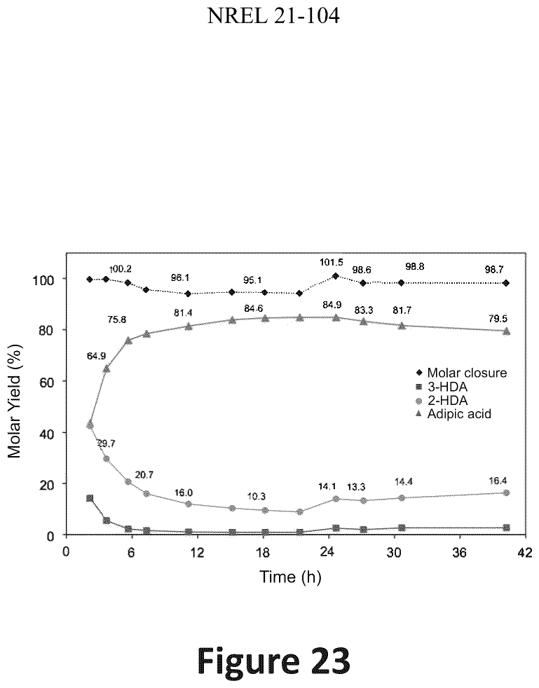

The 100-hour time-on-stream stability test of 1% Rh/AC was then evaluated in a sequential fashion, with partial conversion of muconic acid for the first two days to confirm steady state operation, demonstration of complete conversion to adipic acid for days three and four, and lastly a return to partial conversion conditions on day five to observe any changes compared to the initial reactor performance. Sampling of the reactor was not performed during the first 12 hour overnight, since preliminary experiments showed comparable time was required to reach steady conversion once the liquid feed was introduced (see FIG. 23).

During the first 48-hours of time-on-stream (50° C., 0.5 mL min⁻¹ liquid flow rate), muconic acid was partially converted (57.7±1.9% average molar conversion) to hexenedioic acid (HDA) and adipic acid as the only observed products. Product identities were confirmed by gas chromatography mass spectroscopy. The moderately higher reaction temperature (50° C.) resulted in isomerization to 3-HDA as the predominant species (30.9±1.2% molar yield), in comparison to 2-HDA for room temperature batch screening reactions. Moderate amounts of 2-HDA (19.7±2.9% molar yield) and adipic acid (9.7±1.2% molar yield) were also produced, with an average molar closure of 102.7±4.9%, supporting steady state conversion during the first 48 hours. Variability in molar closure was assumed to be primarily due to solvent evaporation and error introduced during the sampling of knockout pot, with concentrations of individual species throughout the 100-h run reported in Table 4 below.

TABLE 4
Time-on-stream results for the trickle bed hydrogenation of
muconic acid. ª Compounds were monitored by HPLC-RID.

| | | Liq. | | | | |
|---|---|---|---|---|---|---|
| Time | Temp | flow | Muconic | 2-HDA | 3-HDA | Adipic |
| (h) | (° C.) | (mL/min) | (g/L) | (g/L) | (g/L) | (g/L) |
| 18 | 50 | 0.5 | 2.98 | 1.30 | 2.50 | 0.79 |
| 20 | 50 | 0.5 | 3.12 | 1.26 | 2.48 | 0.77 |
| 22 | 50 | 0.5 | 3.20 | 1.16 | 2.35 | 0.72 |
| 24 | 50 | 0.5 | 3.44 | 1.78 | 2.60 | 0.83 |
| 37 | 50 | 0.5 | 3.21 | 1.68 | 2.40 | 0.78 |
| 39 | 50 | 0.5 | 3.36 | 1.64 | 2.34 | 0.74 |
| 41 | 50 | 0.5 | 3.38 | 1.69 | 2.40 | 0.77 |
| 43 | 50 | 0.5 | 3.31 | 1.67 | 2.34 | 0.79 |
| 45 | 50 | 0.5 | 3.31 | 1.65 | 2.34 | 0.75 |
| 67 | 78 | 0.2 | 0.00 | 0.00 | 0.00 | 8.78 |
| 69 | 78 | 0.2 | 0.00 | 0.00 | 0.00 | 7.97 |
| 71 | 78 | 0.2 | 0.00 | 0.00 | 0.00 | 8.14 |
| 73 | 78 | 0.2 | 0.00 | 0.00 | 0.00 | 8.02 |
| 88 | 78 | 0.2 | 0.00 | 0.00 | 0.00 | 7.92 |
| 92 | 78 | 0.2 | 0.00 | 0.00 | 0.00 | 7.97 |
| 96 | 78 | 0.2 | 0.00 | 0.00 | 0.00 | 8.14 |
| 114 | 50 | 0.5 | 3.63 | 1.45 | 1.99 | 0.78 |
| 116 | 50 | 0.5 | 3.70 | 1.80 | 1.91 | 1.00 |
| 118 | 50 | 0.5 | 3.38 | 1.71 | 2.07 | 1.14 |
| 120 | 50 | 0.5 | 3.10 | 1.63 | 2.03 | 0.89 |

ª Reaction conditions were as follows: Muconic acid 1 wt % in ethanol, liquid flow rate and temperature as indicated, H₂ flow 200 sccm, system pressure 24 bar, 1100 mg 1% Rh/AC granules.

Multiple factors can influence the observed reaction rates in trickle bed reactors, including the gas-liquid flow rate ratio, liquid film thickness due to shear, interparticle and intraparticle wetting, and catalyst particle size, shape, and packing geometry. Based on the liquid feed rate flow rate and conversion observed during the first 48 hours, the muconic acid hydrogenation TOF was calculated to be 0.022 sec⁻¹ at 50° C., which was ˜ 1/1000^{th} of the rate observed for powder Rh/AC in batch reactor screening experiments at 24° C. (TOF 7 sec⁻¹), indicating external and intraparticle diffusion likely influenced the observed rate due to larger particle sizes required for trickle-bed reactor experiments. Varying the catalyst bed temperature from 50-72° C. resulted in an apparent activation energy of 60.7 kJ mol⁻¹ for the 1% Rh/AC granule catalyst, well above typical barriers observed under solely mass transfer limiting conditions (<20 kJ mol⁻¹) and comparable to batch reactor results for powder 1% Pd/AC (70 kJ/mol). However, the focus was on examining alterations to the catalyst material properties after time-on-stream rather than a detailed kinetic analysis.

To demonstrate complete conversion of muconic acid to adipic acid, the temperature was increased and liquid flow rate reduced (78° C., 0.2 mL min⁻¹) for day three and four of operation. No peaks from HDA were observed by HPLC-DAD, which was highly sensitive to the presence of olefin bonds, supporting near complete conversion of muconic acid to adipic acid. Lastly, reaction conditions were returned to partial conversion conditions for day five to compare the catalyst conversion and selectivity to the first 48 hour of time-on-stream. Mass balance and product distribution perturbations were observed when altering the liquid flow rate, with a trend toward increasing conversion as time continued. For day five, the average muconic acid molar conversion was 55.2±3.6%, comparable to values observed during the first 48 h of time-on-stream (57.7±1.9%). Product distribution molar yields were also comparable, with average molar closure of 103.5±4.6%.

The following provides further disclosure regarding the solid catalysts describe above for the hydrogenation reaction to convert muconic acid to adipic acid. As used herein, “solid” refers to a solid material that is used as a catalyst and/or as a physical support for one or more catalytic elements. Thus, a solid may provide catalytic activity itself, may provide a structure upon which to build and physically support catalytic elements, or both. Examples of solids used in some embodiments of the present invention include, but are not limited to, carbonaceous materials, oxides, polymers, carbonates, sulfates, and clays. A non-limiting example of a carbonaceous solid is activated carbon. Examples of oxide solids include, but are not limited to, alumina, silica, titanium dioxide, and aluminosilicates. In some embodiments, a carbonaceous material or a silica-containing material may be used as a solid support and/or a solid catalyst for the catalytic conversion of muconic acid to adipic acid.

As used herein, “active site” or “active material” refers to a physical and/or chemical feature that catalyzes a reaction. A catalyst is a substance, structure, element, composition, compound, molecule, or combination thereof that accelerates a chemical reaction without itself being consumed. Examples of active sites include, but are not limited to, one or more elements in their pure form, or in mixtures to form covalently bond molecules, salts, ions, and mixtures thereof. Thus, catalytic active sites may be placed on a solid material. Such active sites may be incorporated into the solid structure itself, for example, by reaction to form covalent bonds that chemically attach at least some of the active sites to the solid. In some embodiments, at least one metal may be combined with a solid to provide a catalyst for the conversion of muconic acid to adipic acid.

In some embodiments, a metallic catalyst comprising a solid may include at least one active site either incorporated into the solid or deposited on the solid, wherein the at least one active site is at least one noble metal, or mixtures thereof. In some cases, a metallic catalyst including a solid may have at least two active sites either incorporated into the solid or deposited on the solid, wherein the at least two active sites are at least two noble metals, or mixtures thereof. As used herein, a “noble metal” refers to at least one of ruthenium, rhodium, palladium, silver, osmium, iridium, platinum, and/or gold. In some further embodiments, a metallic catalyst that includes a solid may have at least one active site either incorporated into the solid or deposited on the solid, wherein the at least one active site is at least one of a noble metal, mercury, rhenium, and/or copper. In some examples, a metallic catalyst including a solid may have at least two active sites either incorporated into the solid or deposited on the solid, wherein the at least two active sites are at least two of a noble metal, mercury, rhenium, and/or copper.

A metallic catalyst that includes a solid may be constructed from solid carbon and at least one of palladium, platinum, and/or ruthenium. At least one metal of a bimetallic catalyst may be at least one of palladium, platinum, and/or ruthenium, may be present in metallic form and/or as a salt. Palladium may be in the 0, +1, +2, +3, +4 oxidation state, or mixtures thereof. Platinum may be in the 0, +1, +2, +3, +4 oxidation state, or mixtures thereof. Ruthenium may be in the −2, 0, +1, +2, +3, +4, +5, +6, +7, +8 oxidation state, or mixtures thereof. Furthermore, a solid used as a support for a metallic catalyst may be at least one of carbon nanotubes, graphene, and/or activated carbon. A bimetallic catalyst may be constructed from intermetallic/core shell nanoparticles. Thus, a bimetallic catalyst comprising two metals and a solid may be utilized to catalyze the hydrogenation reaction of cis,cis-muconic acid with diatomic hydrogen to form at least one of adipic acid, 1,6-hexanediol, or mixtures thereof.

### Example 8: Catalyst Synthesis

Commercial monometallic noble metal catalysts were screened for their hydrogenation activity with muconic acid. Catalysts at 5 wt % loading on activated carbon were obtained from Sigma Aldrich (Pt, Pd, and Ru) and 1 wt % Pd/C was obtained from Alfa Aesar. Virgin catalyst materials were initially characterized to determine their average crystallite size and long-range order by X-ray diffraction, support surface area and pore volume by nitrogen physisorption, and active metal surface area by hydrogen chemisorption, with details described elsewhere. Due to the high sensitivity of Pd dispersion with temperature, Pd samples were reduced under flowing hydrogen (50 mL/min, 10% H₂ in Ar) at moderate temperature (125° C., 3° C./min) and held for 1 hour. Following reduction, Pd samples were purged for 1 hour under Ar and cooled to 45° C. prior to H₂/O₂ titration. For calculations of Pd dispersion, the amount of hydrogen uptake that followed the second oxygen titration was used. A stoichiometry of 0.667 Pd sites per H₂ molecule was assumed to remove oxidized Pd—O species in the form of water and form the reduced Pd—H species.

### Example 9: Catalyst Synthesis

Platinum group metal catalysts (Pt, Rh, Ru, Pd) were synthesized on powder carbon and silica supports to evaluate their activity and stability for muconic acid hydrogenation. For batch reaction studies, Darco activated carbon (Sigma Aldrich) and Davisil Grade 633 high surface area silica (Sigma Aldrich) were used. Supports were initially sieved >270 mesh (<53 m) to minimize the impact of mass transfer on observed kinetics. The silica support was calcined at 500° C. in air prior to loading metals, while the activated carbon support was used as received. Catalysts were prepared with the following metal salt precursors: palladium acetate (Sigma Aldrich), rhodium nitrate hydrate (Sigma Aldrich), ruthenium chloride hydrate (Sigma Aldrich), chloroplatinic acid (CPA) (Sigma Aldrich), and ammonium tetraammineplatinum nitrate (PTA) (Sigma Aldrich). Pd, Ru, and Rh catalysts were prepared by incipient wetness, while Pt catalysts were prepared by strong electrostatic adsorption (SEA) to improve dispersion due to the low activity. For SEA catalyst synthesis, 1.9 g of support was added to 50 mL of DI water, and the pH was adjusted to facilitate protonation/deprotonation of the support (pH 12 with NaOH for silica, pH 2.9 with HCl for AC). In another bottle, the appropriate catalyst precursor was dissolved in 50 mL of DI water (PTA for silica, CPA for activated carbon). The two bottles were mixed together with stirring for 1 hour, followed by vacuum filtration to recover the catalyst. The catalyst was washed twice with 50 mL of DI water and left to dry overnight in air at room temperature. After loading, catalysts were dried at 110° C. and reduced in hydrogen flowing at 200 seem for 2 hours at temperature. Due to the sensitivity of Pd dispersion with temperature, Pd catalysts were reduced at 125° C. while Pt, Rh, and Ru catalysts were reduced at 250° C.

For flow reactor studies, extruded activated carbon pellets (Norit Rx 3 Extra, Cabot Norit) were initially crushed and sieved between 80-100 mesh (150-180 m) to allow for a moderate catalyst bed pressure drop (<5 psig) while still facilitating mass transfer. Rh was loaded onto the support by incipient wetness using ruthenium chloride hydrate (Sigma Aldrich), dried at 110° C., and reduced ex situ prior to use at 250° C. in flowing hydrogen.

Catalysts were characterized after synthesis and reduction to determine their virgin properties, as well as post-reaction for flow reactor experiments. X-ray diffraction (XRD) was used to assess catalyst metal crystallite size and bulk long-range order. Catalyst support surface area, pore volume, and average pore diameter were measured by BET nitrogen physisorption. Scanning electron microscopy, coupled to energy dispersive electron spectroscopy, was used to evaluate of metal crystallite distribution on the support. Chemisorption was used with to evaluate crystallite metal dispersion, defined as the percentage of metal surface sites compared to the total metal loaded.

### Example 10: Catalyst Testing

For batch reactor activity studies, reactions were performed in using a Parrr 5000 Multi-reactor system (Parr Instruments). Commercial cis,cis-muconic acid in the amount of 200 mg (Sigma Aldrich) was dissolved in 19.8 g of 200 proof ethanol. The muconic acid solution and 15 mg of catalyst were then loaded into 75-mL vessels equipped with magnetic stirring. Hydrogenation reactions were performed at 24° C. with hydrogen supplied at a constant 24 bar and stirring at 1600 rpm. Duplication reactions were performed at minimum, with error bars indicating sample standard deviations. Samples were collected via an in situ sample port, syringe filtered, and analyzed by HPLC, as described below. After the reaction, the reactor contents were vacuum filtered (0.2-μm PES filter assembly, Nalgene) to remove catalyst particles, and subsequently the liquid filtrate was analyzed by ICP-OES to examine active metal leaching.

For flow reactor stability studies, reactions were performed using a Parr Tubular reactor system (Parr Instruments) operated in a down-flow trickle-bed configuration. The system was outfitted with a HPLC pump to deliver liquid phase reactants (Series III Scientific Instrument), pair of mass flow controllers to control inert and hydrogen gas delivery (Brooks), tube-in-tube heat exchanger for cooling the reactor effluent, high-pressure 1-L stainless steel knockout pot with bottom sampling valve, and a solenoid-controlled backpressure regulator (Tescom) to maintain system pressure. Reactions were performed in trickle down flow configuration, with gas and liquid reagents fed to through the top of a 32″ long, ¼″ inner diameter stainless steel reaction tube. The tube temperature was monitored and controlled using an internal thermocouple centered in the catalyst bed and three furnace wall thermocouples. The tube was initially packed halfway with inert 1-mm glass beads (Sigma Aldrich) held in place with quartz wool (Quartz Scientific Inc.). The catalyst bed was then loaded at the tube mid-height. Inert quartz sand (Quartz Scientific Inc.) sieved to <60 mesh (>250 μm) was placed at the base and top of the carbon catalyst packing to serve as a support. The remaining reactor tube void was then filled with inert glass beads and sealed with quartz wool.

Continuous hydrogenation reactions were performed with hydrogen supplied at 200 seem and a system pressure maintained at 24 bar. Temperature was varied from 50-78° C., as indicated. The mobile phase consisted of biologically derived muconic acid purified with activated carbon, precipitated by temperature-pH shift crystallization, dissolved in 200-proof ethanol (8 g/L), and filtered (0.2-μm PES) to remove insoluble salts. Commercial succinic acid (Sigma Aldrich, >99.0% reagent purity) was added as an internal standard (0.8 g/L). The liquid flow rate was varied from 0.2-0.5 mL/min, as indicated. Liquid reactor effluent samples collected from the knockout pot were syringe-filtered, and analyzed by HPLC and GC-MS, as described below. Periodically, the liquid filtrate was analyzed by ICP-MS to detect catalyst metal leaching. After testing, the reactor was cooled to room temperature, depressurized, and 500 mL of ethanol was flushed through the catalyst bed, followed by drying under 200 seem nitrogen. The catalyst bed packing solids were then sieved between 80-100 mesh (150-180 m) to recover the catalyst granules for further analysis.

Nylon-6,6 Polymerization with Bio-Adipic Acid

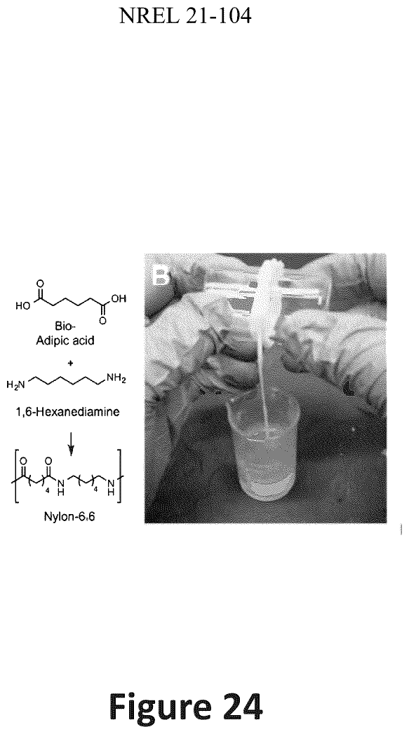

Bio-adipic acid produced from muconic acid was then polymerized with 1,6-hexanediamine to form nylon-6,6 for comparative material testing to petrochemical adipic acid. Bench-scale condensation polymerizations were conducted using the nylon rope reaction shown in FIG. 24. After producing nylon fibers from both bio-adipic acid and commercial adipic acid, polymer materials were dried and characterized to determine their thermal and physicochemical properties.

Thermal analysis of both nylon materials by DSC showed comparable melting and glass transition temperatures, similar to values reported in literature for nylon-6,6, as shown in Table 5 below. Clean thermal traces were observed for nylon produced from biologically derived adipic acid, with a heat of fusion comparable (50.2 J/g) to literature values (51.3 J/g).

Measurement of the intrinsic viscosity by dilute solution viscometry showed similar values for the two nylon materials, and calculations of the viscosity average MW showed that polymerization had taken place to a comparable extent for bio-adipic acid (1,920±20 g/mol) and chemical adipic acid (2,230±40 g/mol). However, the limitation of the nylon rope trick was apparent for achieving industrially relevant nylon MW values (40,000-60,000 g/mol).

TABLE 5
Properties of nylon-6,6 produced using commercial adipic acid
of chemical origin and bio-adipic acid generated in this
work from the catalytic hydrogenation of muconic acid.

| Nylon-6,6 | Adipic Acid | Adipic Acid | Literature for |
|---|---|---|---|
| Properties | Chemical | Biological | Nylon-6,6 |
| Melting Temp | 258 | 264 | 262 |
| (° C.) | | | |
| Glass Transition | 55 | 46 | 50 |
| (° C.)^{a} | | | |
| Heat of fusion | 37.8 | 50.2 | 51.3 |
| (J/g) | | | |
| Crystallinity | 19.9 | 26.4 | 27.0 |
| (%) | | | |
| Intrinsic Viscosity | 26.5 ± 0.9 | 24.1 ± 0.6 | 79-174 |
| (mL/g)^{b} | | | |
| Viscosity Avg. | 2,230 ± 40 | 1,920 ± 20 | 40,000-60,000 |
| MW (g/mol)^{b} | | | |

^{a}T_{g} determined from literature for 27% crystallinity and is known to vary.
^{b}Standard deviation values reported for four solutions tested in triplicate.

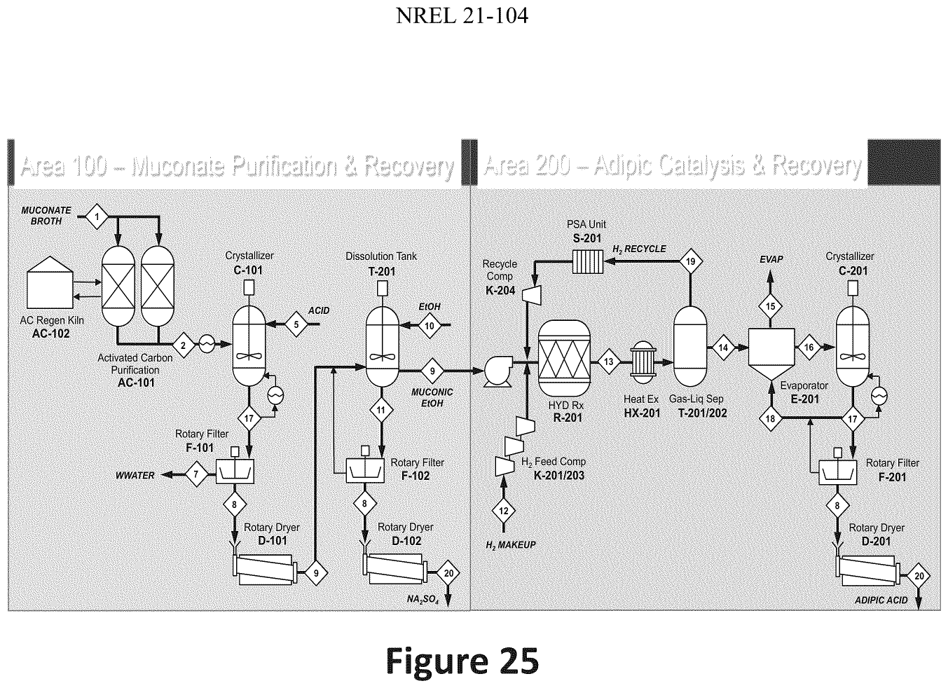

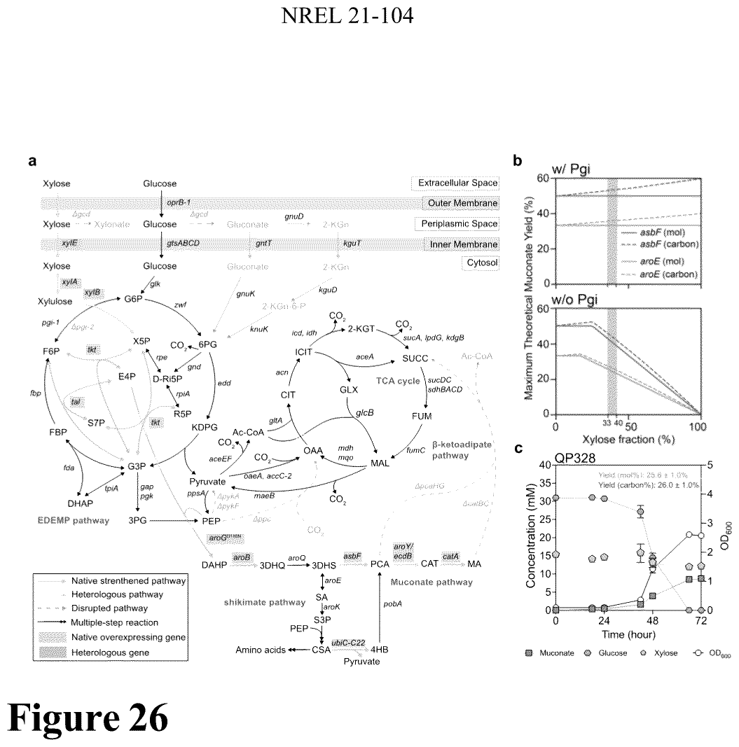

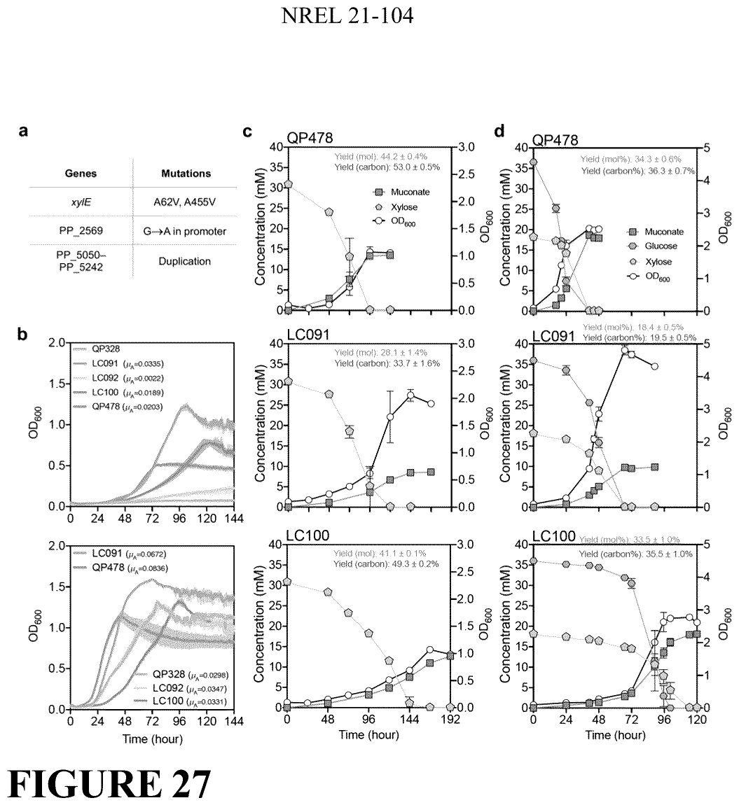

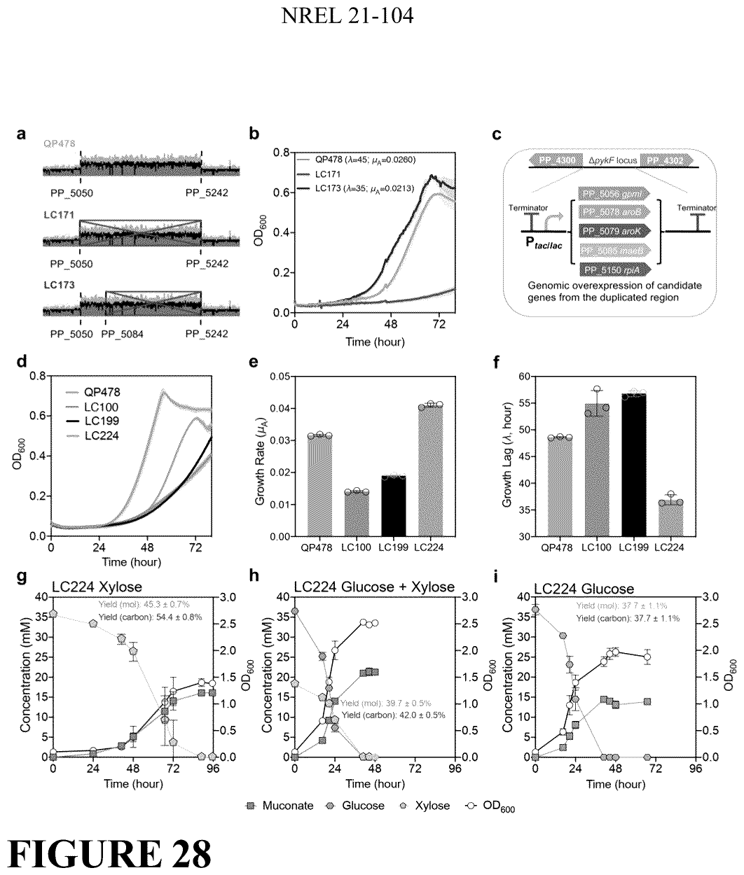

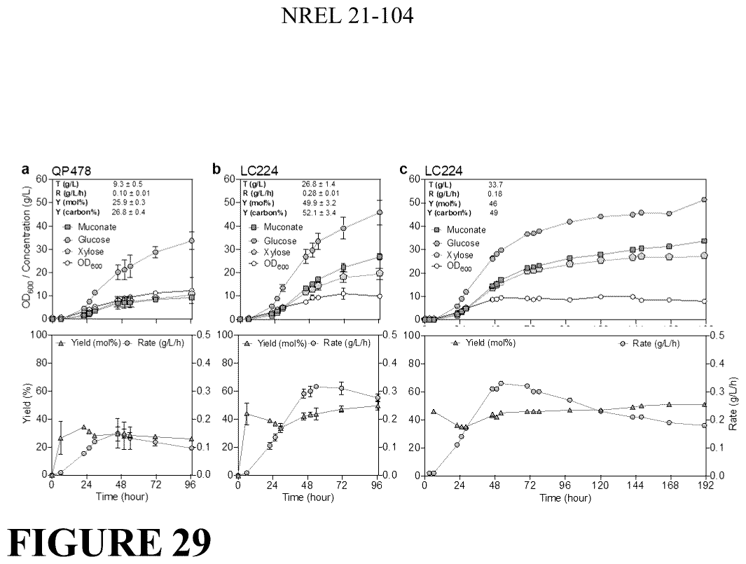

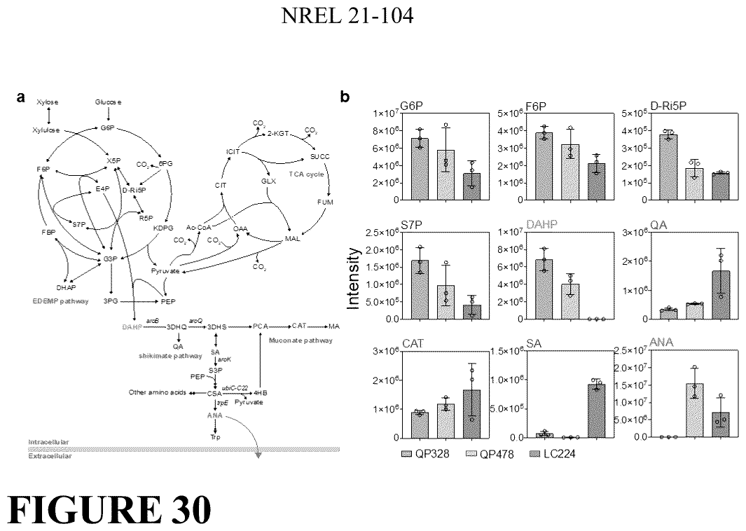

FIG. 25 illustrates a process flow diagram of one example of a portion of a biorefinery, downstream of the biocatalytic bioreactor corresponding to the muconic acid separation/purification and upgrading portions. Thus, FIG. 25 summarizes one hypothetical process and these separation/purification and upgrading steps to convert culture broth from a bioreactor to adipic acid.

Referring to FIG. 25, Area 100 of the process model focuses on purification and recovery of muconic acid from the biological culture broth. Muconic acid broth may be purified over parallel activated carbon treatment beds to remove non-target aromatic impurities. Spent activated carbon from purification may be thermally regenerated onsite by kiln combustion due to the high boiling point of adsorbed organics. After purification, low pH and low temperature crystallization may be employed to recover muconic acid based on a solubility of 3.5 g/L at pH 2 and 10° C. Concentrated sulfuric acid may be used for pH adjustment, producing Na₂SO₄ (2 g of Na₂SO₄ per 1 g of muconic acid solid) that co-crystallizes at a solubility limit of 82.8 g/L at 10° C. Rotary filtration and rotary drying may then be employed to recover mixed solid crystals, with the filter broth effluent treated as wastewater. Mixed solid crystals may be added to ethanol in a heated stirred tank at 70° C. to dissolve muconic acid, while insoluble Na₂SO₄ may be separated by rotary filtration and treated as solid waste.

Area 200 shown in FIG. 25 focuses on the purification and catalytic conversion of muconic acid to adipic acid, with subsequent product recovery. Muconic acid in ethanol may be initially pressurized to 350 psig using a positive-displacement pump for feeding to the reactor. On site hydrogen may be supplied at ambient temperature and pressure, utilizing a 3-stage compression train with inter-stage cooling to deliver hydrogen at 350 psig. Muconic acid, ethanol, and hydrogen may then be mixed and introduced to a trickle bed reactor operating at 70° C. with a weight hour space velocity (WHSV), defined as the weight of liquid solution processed per hour divided by the weight of catalyst material, of 5 h⁻¹. Hydrogenation of muconic acid may be achieved utilizing a 2% Rh/C catalyst, to produce substantially pure adipic acid.

Following hydrogenation, adipic acid may be recovered from solution by ethanol evaporation and crystallization. The stream of adipic acid in ethanol exiting the reactor may be mixed with the crystallizer recycle stream and concentrated to 360 g/L at 82° C., below the adipic acid/ethanol solubility limit of 363 g/L at 60° C. The solution may then be cooled to 10° C. to partially crystallize adipic acid based on a solubility limit of ˜67 g/L at 10° C., with the remaining solution recycled to the inlet of the evaporator. Rotary filtration and drying may then be employed to dry crystals, with an assumed net adipic acid recovery of about 98% post-hydrogenation.

TABLE 6
depicts construction details for plasmids disclosed herein.

| Plasmid | Utility | Construction details |
|---|---|---|
| pK18- | Backbone for gene | ATCC 87097. Previously described in Schäfer, et al., 1994. Genbank: |
| mobsacB | replacement via | FJ437239. |
| | conjugation/electroporation | |
| pK18sB | Backbone for gene | Previously described in Jayakody, et al., 2018. Genbank: MH166772. |
| | replacement via | |
| | electroporation | |
| pK18msB | Backbone for gene | A 244 bp origin of transfer (oriT) was amplified from pK18mobSacB, |
| | replacement via | with primers oCHC-109 and oCHC-110, designed to include overlaps |
| | conjugation/electroporation | for insertion into pK18sB. pK18sB was linearly amplified with oCHC- |
| | | 107 and oCHC-108, generating a 3038 bp backbone. The oriT |
| | | fragment was inserted to the backbone using Gibson assembly. |
| pCJ075 | Backbone for constructing | Previously described in Johnson, et al., 2019. |
| | plasmid pLC028 | |
| pCJ097 | Deletion of pgi-1 | Previously described in Johnson, et al., 2019. |
| pCJ098 | Deletion of pgi-2 | Previously described in Johnson, et al., 2019. |
| pJE443 | Insertion of the xylose | Previously described in Elmore, et al., 2020. |
| | isomerase pathway | |
| | (P_{xylE}:xylE:P_{tac}:xylAB:talB:tktA) | |
| | at the ΔampC locus | |
| pJE1369 | Deletion of hexR in strain CJ522 | Previously described in Elmore, et al., 2020. |
| pQP154 | Insertion of ubiC-C22 into the | A fragment of 2952 bp for introducing ubiC-C22 was amplified from |
| | genome of P. putida, between | the plasmid pPobR_ubiC-C22⁶ using primers oQP-430 and oQP-433. |
| | PP_1737 and PP_1736 | The vector backbone was amplified from plasmid pK18mobsacB |
| | | using primers oQP-519 and oQP-520. The ubiC fragment was then |
| | | inserted to the vector backbone using Gibson assembly. |
| pQP150 | Restoration of pgi-1 | A fragment of 3317 bp containing pgi-1 and flanking targeting |
| | | regions was amplified from genomic DNA of P. putida KT2440 using |
| | | primers oQP-521 and oQP-522. The fragment was then inserted into |
| | | the EcoRI HindIII digest of pK18mobsacB plasmid by ligation. |
| pLC009 | Restoration of A62V and A455V | A fragment of 3112 bp for restoring xylE was amplified from |
| | in XylE in strain QP478 | genomic DNA of QP328 using primers oLC-0041 and oLC-0044. The |
| | | backbone was amplified from pK18sb using primers oLC-0005 and |
| | | oLC-0006. The fragment was inserted to the backbone using Gibson |
| | | assembly. |
| pLC010 | Introducing A62V and A455V in | A fragment of 3112 bp for introducing mutations to xylE was |
| | XylE in strain QP328 | amplified from genomic DNA of QP328 using primers oLC-0041 and |
| | | oLC-0044. The backbone was amplified from pK18sb using primers |
| | | oLC-0005 and oLC-0006. The fragment was inserted to the |
| | | backbone using Gibson assembly. |
| pLC011 | Restoration of the G→A | A fragment of 3397 bp for restoring the G→A mutation in the |
| | mutation in the promoter | promoter region of PP_2569 was amplified from genomic DNA of |
| | region of PP_2569 in strain | QP328 using primers oLC-0037 and oLC-0040. The backbone was |
| | QP478 | amplified from pK18sb using primers oLC-0005 and oLC-0006. The |
| | | fragment was inserted to the backbone using Gibson assembly. |
| pLC016 | Restoration of A62V in XylE | The plasmid was synthesized by Twist Bioscience. The sequence is |
| | from strain QP478; also used to | attached below in Supplementary Table 8 as tLC-002. |
| | introduce the A455V mutation | |
| | in XylE to strain QP328 | |
| pLC017 | Restoration of A455V in XylE | The plasmid was synthesized by Twist Bioscience. The sequence is |
| | from strain QP478; also used to | attached below in Supplementary Table 8 as tLC-003. |
| | introduce the A62V mutation in | |
| | XylE to strain QP328 | |
| pLC025 | Introducing the G→A mutation | A fragment of 1418 bp for introducing G→A mutation to promoter |
| | in the promoter region of | region of PP_2569 was amplified from genomic DNA of QP478 using |
| | PP_2569 to strain QP328 and | primers oLC-0181 and oLC-0182. The backbone was amplified from |
| | LC091 | pK18sb using primers oLC-0005 and oLC-0006. |
| pLC028 | Plasmid for gene insertion at | The backbone including the upstream or downstream targeting |
| | ΔpykF locus driven by P_{lac} | region was amplified from plasmid pCJ075 using primers oLC-0196 |
| | promoter | and oLC-0197 (pCJ075 was used for deleting pykF in P. putida). The |
| | | fragment containing terminators and the P_{lac} promoter was |
| | | amplified from plasmid pQP154 using oLC-0195 and oLC-0198. The |
| | | fragment was inserted to the backbone using Gibson assembly. |
| | | After Sanger sequencing, a 104 bp deletion in the homologous arm |
| | | downstream ΔpykF (PP_4301) locus was identified in the plasmid. |
| | | The deletion also exits in pLC028 derived plasmids pLC030, pLC038, |
| | | pLC042, pLC043, pLC056, and pLC067, according to Sanger |
| | | sequencing. The deletion is 107 bp upstream of coding sequence of |
| | | PP_4302. To evaluate the potential effect of the deletion on |
| | | expression of PP_4302, we performed RT-qPCR of the gene |
| | | PP_4302 in strains LC100 and LC224 (LC100 ΔpykF::P_{tac}:aroB). The |
| | | results demonstrated that the deletion had no significant effect on |
| | | the expression level of PP_4302 (Supplementary FIG. 9). Details of |
| | | the RT-qPCR can be found in Supplementary Table 4. The sequence |
| | | of pLC028 was listed in Supplementary Table 8. |
| pLC030 | Genomic overexpression of | A fragment of 1601 bp for introducing PP_5056 was amplified from |
| | PP_5056 (gpmI) at the ΔpykF | the genomic DNA of QP328, using primers oLC-0203 and oLC-0204. |
| | locus in strain LC100 | The backbone was amplified from pLC028 using oLC-0199 and oLC- |
| | | 0200. The fragment was inserted to the backbone using Gibson |
| | | assembly. |
| pLC038 | Plasmid for gene insertion at | P_{lac} promoter in pLC028 was replaced with P_{tac} promoter. The |
| | ΔpykF locus driven by P_{tac} | backbone was amplified from plasmid pLC028 using primers oLC- |
| | promoter | 0199 and oLC-0218. The fragment containing P_{tac} promoter was |
| | | amplified from pQP154 using primers oLC-0217 (containing |
| | | sequence to introduce the P_{tac} promoter) and oLC-0220. The |
| | | fragment was inserted to the backbone using Gibson assembly. |
| pLC042 | Genomic overexpression of | A fragment of 1402 bp for introducing PP_5085 was amplified from |
| | PP_5085 (maeB) at the ΔpykF | the genomic DNA of QP328, using primers oLC-0205 and oLC-0206. |
| | locus in strain LC100 | The backbone was amplified from pLC038 using oLC-0199 and oLC- |
| | | 0219. The fragment was inserted to the backbone using Gibson |
| | | assembly. |
| pLC043 | Genomic overexpression of | A fragment of 741 bp for introducing PP_5150 was amplified from |
| | PP_5150 (rpiA) at the ΔpykF | the genomic DNA of QP328, using primers oLC-0203 and oLC-0204. |
| | site in strain LC100 | The backbone was amplified from pLC038 using oLC-0199 and oLC- |
| | | 0219. The fragment was inserted to the backbone using Gibson |
| | | assembly. |
| pLC052 | Deletion of PP_5050- | The 747 bp homology arm upstream PP_5050 was amplified from |
| | PP_5242 from strain QP478 | QP478 gDNA with primers oLC-0235 and oLC-0236, which contain |
| | | overlaps to the pK18msB backbone and the homology arm |
| | | downstream of PP_5242, respectively. The 747 bp downstream |
| | | homology arm was amplified from QP478 gDNA with primers oLC- |
| | | 0237 and oLC-0238, which include overlaps to the upstream |
| | | homology arm and the pK18msB backbone, respectively. The |
| | | backbone was amplified from pK18msB using primers oLC-0005 and |
| | | oLC-0006. The two homologous arms were inserted to the |
| | | backbone using Gibson assembly. |
| pLC053 | Deletion of PP_5084- | The 761 bp homology arm upstream PP_5084 was amplified from |
| | PP_5242 from strain QP478 | QP478 gDNA with primers oLC-0239 and oLC-0240 which contain |
| | | overlaps to the pK18msB backbone and the homology arm |
| | | downstream PP_5242, respectively. The 747 bp downstream |
| | | homology arm was amplified from QP478 gDNA with primers oLC- |
| | | 0237 and oLC-0238 which include overlaps to the upstream |
| | | homology arm and the pK18msB backbone, respectively. The |
| | | backbone was amplified from pK18msB using primers oLC-0005 and |
| | | oLC-0006. The two homologous arms were inserted to the |
| | | backbone using Gibson assembly. |
| pLC056 | Genomic overexpression of | A fragment of 1788 bp containing PP_5079 and PP_5078 was |
| | PP_5079 (aroK) and PP_5078 | amplified from the genomic DNA of QP328, using primers oLC-0244 |
| | (aroB) in an operon at the | and oLC-0246. The backbone was amplified from pLC038 using oLC- |
| | ΔpykF locus | 0199 and oLC-0219. The fragment was inserted to the backbone |
| | | using Gibson assembly. |
| pLC067 | Genomic overexpression of | A fragment of 594 bp containing PP_5079 was amplified from the |
| | PP_5079 (aroK) at the ΔpykF | genomic DNA of QP328, using primers oLC-0244 and oLC-0280. The |
| | locus | backbone was amplified from pLC038 using oLC-0199 and oLC- |
| | | 0219. The fragment was inserted to the backbone using Gibson |
| | | assembly. |
| pLC068 | Genomic overexpression of | A fragment of 1234 bp containing PP_5079 was amplified from the |
| | PP_5078 (aroB) at the ΔpykF | genomic DNA of QP328, using primers oLC-0246 and oLC-0281. The |
| | locus | backbone was amplified from pLC038 using oLC-0199 and oLC- |
| | | 0219. The fragment was inserted to the backbone using Gibson |
| | | assembly. |
| pLC088 | Deletion of PP_5079 (aroK) at | The plasmid was synthesized by Twist Bioscience. The sequence is |
| | the ΔpykF locus from strain | attached below in the Supplementary Table 8 as tLC-008. |
| | LC168 | |

TABLE 8
depicts construction details of strains disclosed herein.

| Strain | Genotype | Construction details |
|---|---|---|
| CJ522 | P. putida KT2440 | Previously described in Johnson, et al., 2019.⁵ |
| | ΔcatRBC::Ptac:catA | |
| | ΔpcaHG::Ptac:aroY:ecdB:asbF | |
| | ΔpykA::aroG-D146N:aroY:ecdB:asbF | |
| | ΔpykF Δppc Δpgi-1 Δpgi-2 Δgcd | |
| JE3226 | P. putida KT2440 | Previously described in Elmore, et al., 2020.¹ |
| | ΔhsdR::Ptac:BxB1int-attB Δgcd | |
| | ΔampC::xylE A insertion point | |
| | mutation of PxylE:xylAB:tktA:talB | |
| LC041 | JE3226 Δpgi-1 | Deletion of pgi-1 in JE3226 was performed using plasmid pCJ097. |
| | | Deletion was determined by colony PCR using primers oLC-0071 and |
| | | oLC-0072 generating a band of 3779 bp for WT and a band of 2099 |
| | | bp for the deletion. |
| LC345 | JE3226 Δpgi-2 | Deletion of pgi-2 in JE3226 was performed using plasmid pCJ098. |
| | | Deletion was determined by colony PCR using primers oLC-0469 and |
| | | oLC-0470 generating a band of 3765 bp for WT and a band of 2092 |
| | | bp for the deletion. |
| LC347 | LC041 Δpgi-2 | Deletion of pgi-2 in LC041 was performed using plasmid pCJ098. |
| | | Deletion was determined by colony PCR using primers oLC-0469 and |
| | | oLC-0470 generating a band of 3765 bp for WT and a band of 2092 |
| | | bp for the deletion. |
| QP328 | CJ522 ΔhexR | hexR was deleted in strain CJ522 using plasmid pJE1369, and the |
| | ΔampC::P_{xylE}:xylE:P_{tac}:xylAB:talB:tktA | deletion was confirmed using flanking primers oJE-1365 and oJE- |
| | Δpgi-1::pgi-1 PP_1736- | 1366, and internal primers oJE-1367 and oJE-1368. The xylose |
| | 1737(intergenic)::Plac:ubiC-C22 | isomerase pathway including genes xylE-xylAB:talB:tktA was |
| | | inserted to the genome at the ΔampC locus using plasmid pJE443 |
| | | and the insertion was confirmed by colony PCR with primers oQP- |
| | | 2172 and oQP-036. ubiC-C22 was inserted between PP_1737 and |
| | | PP_1736 using plasmid pQP154 and the insertion was confirmed by |
| | | colony PCR using primers oQP-350 and oQP-351. pgi-1 was then |
| | | restored using plasmid pQP150 and confirmed by colony PCR with |
| | | oQP-525 and oQP-526. |
| QP478 | ALE isolate #2: | QP328 was evolved on M9 medium supplemented with 10 mM |
| | QP328 xylE-A62V, A455V P_{PP}_{_{—}}₂₅₆₉ | xylose. Isolate 2 performed the best and was named QP478. |
| | G→A duplication of PP_5050- | |
| | PP_5242 | |
| LC061 | QP478 with WT restoration of | The G→A mutation in P_{PP}_{_{—}}₂₅₆₉ was restored to the WT sequence in |
| | G→A at P_{PP}_{_{—}}₂₅₆₉ | strain QP478 using plasmid pLC011. Correct restorations were |
| | | confirmed by Sanger sequencing of the PCR product amplified by |
| | | primers oLC-0025 and oLC-0026, using oLC0025 as the sequencing |
| | | primer. |
| LC075 | QP478 with WT restoration of | The A62V and A455V mutations in xlyE were restored to the WT |
| | xylE-A62V, A455V | sequence in strain QP478 using plasmid pLC009. Single colonies |
| | | were first pre-screened by colony PCR (58° C., 2 min, 28 cycles) using |
| | | primers oLC-0127 and oLC-0102. Those with lighter bands tended |
| | | to be correct mutants and were further confirmed by Sanger |
| | | sequencing of the PCR products amplified by primers oLC-0027 and |
| | | oLC-0028, using oLC0027 and oLC-0028 as the sequencing primer. |
| LC078 | QP478 with WT restoration of | The A455V mutation in xlyE were restored using plasmid pLC017. |
| | xylE-A455V | Correct restorations were confirmed by Sanger sequencing of the |
| | | PCR products amplified by primers oLC-0027 and oLC-0028, using |
| | | oLC0027 as the sequencing primer. |
| LC091 | QP328 xylE-A62V, A455V | The A62V and A455V mutations in xlyE were introduced into QP328 |
| | | using plasmid pLC010. Correct restorations were confirmed by |
| | | Sanger sequencing of the PCR products amplified by primers oLC- |
| | | 0027 and oLC-0028, using oLC-0027 and oLC-0028 as the |
| | | sequencing primers. |
| LC092 | QP328 P_{PP}_{_{—}}₂₅₆₉ G→A | The G→A mutation in P_{PP}_{_{—}}₂₅₆₉ was introduced into QP328 using |
| | | plasmid pLC025. Correct restorations were confirmed by Sanger |
| | | sequencing of the PCR product amplified by primers oLC-0025 and |
| | | oLC-0026, using oLC-0025 as the sequencing primer. |
| LC093 | QP478 with WT restoration of | The A455V mutation in xlyE was restored using plasmid pLC016. |
| | xylE-A62V | Correct restorations were confirmed by Sanger sequencing of the |
| | | PCR products amplified by primers oLC-0027 and oLC-0028, using |
| | | oLC-0028 as the sequencing primer. |
| LC100 | LC092 xylE-A62V, A455V | The A62V and A455Vmutations in xlyE were introduced into LC092 |
| | | using plasmid pLC010. Correct restorations were confirmed by |
| | | Sanger sequencing of the PCR products amplified by primers oLC- |
| | | 0027 and oLC-0028, using oLC-0027 and oLC-0028 as the |
| | | sequencing primers. |
| LC147 | LC100 ΔpykF::P_{lac}:gpmI | A fragment overexpressing PP_5056 (gpmI) driven by P_{lac} promoter |
| | | was inserted to the genome of LC100 using the plasmid pLC030. The |
| | | insertion was confirmed by colony PCR with primers oLC-0231 and |
| | | oLC-0232 generating bands of 1585 bp for WT and 3331 bp for the |
| | | replacement. |
| LC150 | LC100 ΔpykF::P_{tac}:maeB | A fragment overexpressing PP_5085 (maeB) driven by P_{tac} promoter |
| | | was inserted to the genome of LC100 using the plasmid pLC042. The |
| | | insertion was confirmed by colony PCR with primers oLC-0231 and |
| | | oLC-0232 generating bands of 1585 bp for WT and 3132 bp for the |
| | | replacement. |
| LC151 | LC100 ΔpykF:P_{tac}:rpiA | A fragment overexpressing PP_5150 (rpiA) driven by P_{tac} promoter |
| | | was inserted to the genome of LC100 using the plasmid pLC043. The |
| | | insertion was confirmed by colony PCR with primers oLC-0231 and |
| | | oLC-0232 generating bands of 1585 bp for WT and 2471 bp for the |
| | | replacement. |
| LC168 | LC100 ΔpykF:P_{tac}:aroK:aroB | A fragment overexpressing an operon containing genes PP_5079 |
| | | (aroK) and PP_5078 (aroB) driven by Ptac promoter was inserted to |
| | | the genome of LC100 using the plasmid pLC056. The insertion was |
| | | confirmed by colony PCR with primers oLC-0231 and oLC-0232 |
| | | generating bands of 1585 bp for WT and 3501 bp for the |
| | | replacement. |
| LC171 | QP478 ΔPP_5050-PP_5242 | Deletion of PP_5050-PP_5242 was performed using plasmid |
| | | pLC052. Deletion was determined by colony PCR using primers oLC- |
| | | 0242 and oLC-0243 generating no band for WT (too large) and a |
| | | band of 1562 bp for the deletion. |
| LC173 | QP478 ΔPP_5084-PP 5242 | Deletion of PP_5050-PP_5242 was performed using plasmid |
| | | pLC053. Deletion was determined by colony PCR using primers oLC- |
| | | 0242 and oLC-0241 generating no band for WT (too large) and a |
| | | band of 1595 bp for the deletion. |
| LC199 | LC100 ΔpykF::P_{tac}:aroK | A fragment overexpressing PP_5079 (aroK) driven by P_{tac} promoter |
| | | was inserted to the genome of LC100 using the plasmid pLC067. The |
| | | insertion was confirmed by colony PCR with primers oLC-0231 and |
| | | oLC-0232 generating bands of 1585 bp for WT and 2307 bp for the |
| | | replacement. |
| LC224 | LC100 ΔpykF::P_{tac}:aroB | aroK was deleted from the genome of strain LC168 using plasmid |
| | | pLC088. Deletion was determined by colony PCR using primers oLC- |
| | | 0231 and oLC-0232 generating bands of 3501 bp for WT and 2947 |
| | | bp for the replacement. |
| LC349 | QP328 ΔpykF:P_{tac}:aroB | A fragment overexpressing PP_5078 (aroB) driven by P_{tac} promoter |
| | | was inserted to the genome of QP328 using the plasmid pLC068. |
| | | The insertion was confirmed by colony PCR with primers oLC-0231 |
| | | and oLC-0232 generating bands of 1585 bp for WT and 2947 bp for |
| | | the replacement. |

TABLE 9
depicts oligonucleotide sequences disclosed herein.

| SEQ ID NO: | Oligo | Sequence (5′-3′) |
|---|---|---|
| SEQ ID NO: 19 | oCHC-107 | AGATCAAAGGATCTTCTTGAGATCCTTTTTTTCTGCGC |
| SEQ ID NO: 20 | oCHC-108 | TTTCTACGGGGTCTGATTATTTGTTAACTGTTAATTGTCCTTG |
| SEQ ID NO: 21 | oCHC-109 | AACAAATAATCAGACCCCGTAGAAACGGCCAGCCTCGCAGAGCAG |
| SEQ ID NO: 22 | oCHC-110 | GGATCTCAAGAAGATCCTTTGATCTCTTTTCCGCTGCATAACCCTGCTTCGG |
| SEQ ID NO: 23 | oJE-1365 | CATTCGAGTGGGAGCGTTAT |
| SEQ ID NO: 24 | oJE-1366 | TTCTGGTTCCACTGGGTTTC |
| SEQ ID NO: 25 | oJE-1367 | CGAGGTCATTGGCATGTAGA |
| SEQ ID NO: 26 | OJE-1368 | CGAAGTCATCCTGCTCAACC |
| SEQ ID NO: 27 | oQP-036 | CGATACGGTGTGCCACAAACC |
| SEQ ID NO: 28 | oQP-350 | GCGTTCATCCGTCATCCACTC |
| SEQ ID NO: 29 | oQP-351 | TTTGCAAGCAGCAGATTACG |
| SEQ ID NO: 30 | oQP-430 | CGTTGTAAAACGACGGCCAGTGCCAAGCTTGTGATGCACAACGCCGAC |
| SEQ ID NO: 31 | oQP-433 | GCTATGACATGATTACGAATTCCATCAACGCCGTCACCC |
| SEQ ID NO: 32 | oQP-519 | TACACTTTATGCTTCCGGCTCGTATGTTGTGGTGATCAGACCTGGAATTGTGAG |
| SEQ ID NO: 33 | oQP-520 | CACAACATACGAGCCGGAAGCATAAAGTGTAAAGCGTTTATTTTGAAGTTTTTTGCGAG |
| SEQ ID NO: 34 | OQP-521 | AGGAAACAGCTATGACATGATTACGAATTCCTGGTCATTACCCGCTTCCTTG |
| SEQ ID NO: 35 | oQP-522 | CGTTGTAAAACGACGGCCAGTGCCAAGCTTCTGTCTATAGGGTGATGCCGTAACA |
| SEQ ID NO: 36 | oQP-525 | GGGTTCCAGCTTGTCCAATGG |
| SEQ ID NO: 37 | oQP-526 | CCAACGGTAGAAGGTAACTGTG |
| SEQ ID NO: 38 | oQP-2172 | GAAGATGTAGCTGCTGTTATACTG |
| SEQ ID NO: 39 | oLC-0005 | ACTGGCCGTCGTTTTACAACGT |
| SEQ ID NO: 40 | oLC-0006 | GTCATAGCTGTTTCCTGTGTGAGGCGT |
| SEQ ID NO: 41 | oLC-0025 | CAACCTGCACCATTTCTGCCT |
| SEQ ID NO: 42 | oLC-0026 | GTGTACTTGCGCCCGATCATAT |
| SEQ ID NO: 43 | oLC-0027 | CCGACAATCAACGCGAGCGT |
| SEQ ID NO: 44 | oLC-0028 | TGCATATGATATCTCCTGTGTGACTT |
| SEQ ID NO: 45 | oLC-0037 | ACACAGGAAACAGCTATGACTGCCCGTTGTACCTGCTGTT |
| SEQ ID NO: 46 | oLC-0040 | GTTGTAAAACGACGGCCAGTTGGAGCAGCAAATTGCAGATTGTGACG |
| SEQ ID NO: 47 | oLC-0041 | ACACAGGAAACAGCTATGACCTTGCCTCTGCCGGAAACCC |
| SEQ ID NO: 48 | oLC-0044 | GTTGTAAAACGACGGCCAGTCCTGCCGCAAATCGGTATTC |
| SEQ ID NO: 49 | oLC-0107 | TTTACCGCAACCCTGAGTG |
| SEQ ID NO: 50 | oLC-0108 | GGTCACGTAGATCACCAGAATG |
| SEQ ID NO: 51 | oLC-0109 | AGGCATTCGTGAAGTCATGG |
| SEQ ID NO: 52 | oLC-0110 | ATGTAACCGCTGAGAACGTC |
| SEQ ID NO: 53 | oLC-0181 | ACACAGGAAACAGCTATGACGGCACCACTTCCACCTGGAT |
| SEQ ID NO: 54 | oLC-0182 | GTTGTAAAACGACGGCCAGTAGGGTGGCAGTCGTCTGGAT |
| SEQ ID NO: 55 | oLC-0195 | GCACACCGTCAACCATTGAGCCT |
| SEQ ID NO: 56 | oLC-0196 | CTCAATGGTTGACGGTGTGCTCATGATTGGGCAGTCTCAA |
| SEQ ID NO: 57 | oLC-0197 | CTTGTGTTGCTCCCATTCGCGATTGCCGGGGGCGCGTT |
| SEQ ID NO: 58 | oLC-0198 | GCGAATGGGAGCAACACAAG |
| SEQ ID NO: 59 | oLC-0199 | GATATGGATCCATCAATAAAACGAAAGGCT |
| SEQ ID NO: 60 | oLC-0200 | AATTGTTATCCGCTCACAATTCCAGGT |
| SEQ ID NO: 61 | oLC-0203 | ATTGTGAGCGGATAACAATTCTTTGAACAACCGTGAGTTTGATTG |
| SEQ ID NO: 62 | oLC-0204 | TTTATTGATGGATCCATATCTCAGGCGTCGACCAGAATCG |
| SEQ ID NO: 63 | oLC-0205 | ATTGTGAGCGGATAACAATTCGTAGGCTAAAGGCCTTGACAC |
| SEQ ID NO: 64 | oLC-0206 | TTTATTGATGGATCCATATCTCAGCCGTTGAACACTTCAT |
| SEQ ID NO: 65 | oLC-0217 | AATCATCGGCTCGTATAATGTGTGGAATTGTGAGCGGATAACAATT |
| SEQ ID NO: 66 | oLC-0218 | CATTATACGAGCCGATGATTAATTGTCAAGCGTTTATTTTGAAGTTTTTTGCG |
| SEQ ID NO: 67 | oLC-0219 | AATTGTTATCCGCTCACAATTCCAC |
| SEQ ID NO: 68 | oLC-0220 | TTTATTGATGGATCCATATCCCCAATTTTTGTAACGCCAGCT |
| SEQ ID NO: 69 | oLC-0231 | CGCAAGCACCTCTCGGCGAT |
| SEQ ID NO: 70 | oLC-0232 | GCCGCATAGGGATTGACGAT |
| SEQ ID NO: 71 | oLC-0235 | ACACAGGAAACAGCTATGACTGCTGAACCTGCCACACGAT |
| SEQ ID NO: 72 | oLC-0236 | TACGGTGGTGGCGGCAACGCT |
| SEQ ID NO: 73 | oLC-0237 | GCGTTGCCGCCACCACCGTAGAGGCCAGTACAGGTTTACAT |
| SEQ ID NO: 74 | oLC-0238 | GTTGTAAAACGACGGCCAGTCCTGGCGGCAGGCGCAGAC |
| SEQ ID NO: 75 | oLC-0239 | ACACAGGAAACAGCTATGACTTGATACGTCTGCTGAAGTTC |
| SEQ ID NO: 76 | oLC-0240 | GATAGCTGGCGTTGAGCGGCT |
| SEQ ID NO: 77 | oLC-0241 | GGTCCACCGGACCTGCGTG |
| SEQ ID NO: 78 | oLC-0242 | CGTGCTTTCAGCCGCCTGGT |
| SEQ ID NO: 79 | oLC-0243 | GAAGTGCTGATCGGCGACCT |
| SEQ ID NO: 80 | oLC-0244 | ATTGTGAGCGGATAACAATTGAGTGACCAGGCGATTGCTG |
| SEQ ID NO: 81 | oLC-0246 | TTTATTGATGGATCCATATCTCAAAGCTGGGCCACGATC |
| SEQ ID NO: 82 | oLC-0263 | ACACAGGAAACAGCTATGACCTCTTGTAATGCATCGCGT |
| SEQ ID NO: 83 | oLC-0264 | TGGAGGGTGACGGCATCCCT |
| SEQ ID NO: 84 | oLC-0280 | TTTATTGATGGATCCATATCTTAACGGGGCGGCAACTGCT |
| SEQ ID NO: 85 | OKC-0281 | ATTGTGCGGATACTCGGCCT |
| SEQ ID NO: 86 | oLC-0353 | CTACACCTCCCCTTTCGTATTG |
| SEQ ID NO: 87 | oLC-0354 | TGGTCGAGCAGGAAAATCTG |

TABLE 10
depicts sequences of plasmids disclosed herein.

tLC-002 (pLC016) Upstream homologous arm, genome edit, downstream homologous arm (SEQ ID NO: 88)
TTGAGATCCTTTTTTTCTGCGCGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCTCACACAGGAAACAGCTATGACATGATTACGAATTCTAATACAACACCACG
TTGATCCCGACGAACTGCTGGAAAATGGACAACATCACGCCAATCACGATCACCCCCACGCCGAACATCAGCAAGCGCCCG
CCGGTCTTGCGGCCGTGGTCCAAGGAGTGTTTGATCTCTTGCACGGCCTGCGTGGCCAGGGTATTGCCCATGATCTTCCGC
AAGATGCCTTCGGCTTGTTCCTGCTTGCCACGGCTCATCAGCCAGCGCGGGCTCTCGGGGACGGTGTACAGCAGCATCAGG
AACAGCAGCGCTGGAATACATTCGGACGCGAACATGTAGCGCCAACCATCGGTATTCAGCCAGCTGGCATCGCCCGAACG
CGCAATGAAGTAATTCACGCAGTACACCAACAGCTGCCCAAAGATGATCGCGAACTGGTTGAAGCTGACCAGCTTCCCCCG
GATATGGGCGGGCGCCAATTCGGCAATGTACATCGGCGACAACATGGAGGCCAGGCCGACCCCGATCCCACCGATGATGC
GGTAGATCACGAACTCGGGCACGTAACCGGCCAGATAGACTGGGACGGTGTTGTCCGGGTTAATCGAGGTGAAACCCAGT
TCCGGCCACGCGGACCCCACACCGCTGATAAAAAACAAGACGGCCGCGATCTTCAAGCTGTCGCGGCGCCCGAAGCGGTT
GCTGCAATAGCCGCCCAGGGCCCCGCCGATGATGCAGCCGATCAGGGCGCTCGCCACGCAGAAGCCCAGCAGGCTGTTGG
CCGCCGACTCCGACAGGTTCTGCGGGGCCACGAACACGGTGTTCAGGCTTTCCACGGTGCCGCTGATCACGGCGGTATCAT
AACCGAACAGCAGGCCGCCCAGGGTGGCCACCAAGGTGATGCTGAAGATGTAGCTGCTGTTATACTGGGTGTTCATAAGA
AATCTCCTTATGCTTAAGTTGTCTTCATGCTTCCACACAACATACGAGCCGGAAGCATAAAGTCTAAGGCCTGGAAGACGCA
GACATTCCATACTAGTTTATGCCATGTCTTGAGACCTTGACAATTAATCATCGGCTCGTATAATGTGTGGAATTGTGAGCGG
ATAACAATTGGTCTCACTTAAGTCACACAGGAGATATCATATGCAGGCCTACTTCGACCAGCTGGACCGCGTGCGCTACGA
AGGCAGCAAATCCTCGAACCCGCTGGCCTTCCGTCACTACAACCCAGACGAGTTGGTGCTGGGTAAGCGCATGGAGGAGC
ATCTGCGCTTCGCCGCCTGCTATTGGCACACGTTCTGCTGGAACGGCGCGGACATGTTCGGCGTCGGCGCGTTTAACCGCC
CCTGGCAGCAGCCGGGCGAGGCCCTGGCGCTGGCGAAGCGCAAGGCGGACGTGGCCTTCGAGTTCTTCCATAAACTGCAC
GTCCCATTCTATTGCTTCCATGACGTGGACGTGAGCCAAGCTTGGCACTGGCCGTCGTTTTACAACGTCGTGACCGGAATTG
CCAGCTGGGGCGCCCTCTGGTAAGGTTGGGAAGCCCTGCAAACAGGATGAGGATCGTTTCGCATGATTGAACAAGATGGA
TTGCACGCAGGTTCTCCGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGTGCCCTGAATGAACTC
CAAGACGAGGCAGCGCGGCTATCGTGGCTGGCCACGACGGGCGTTCCTTGCGCAGCTGTGCTCGACGTTGTCACTGAAGC
GGGAAGGGACTGGCTGCTATTGGGCGAAGTGCCGGGGCAGGATCTCCTGTCATCTCACCTTGCTCCTGCCGAGAAAGTAT
CCATCATGGCTGATGCAATGCGGCGGCTGCATACGCTTGATCCGGCTACCTGCCCATTCGACCACCAAGCGAAACATCGCA
TCGAGCGAGCACGTACTCGGATGGAAGCCGGTCTTGTCGATCAGGATGATCTGGACGAAGAGCATCAGGGGCTCGCGCCA
GCCGAACTGTTCGCCAGGCTCAAGGCGCGGATGCCCGACGGCGAGGATCTCGTCGTGACCCATGGCGATGCCTGCTTGCC
GAATATCATGGTGGAAAATGGCCGCTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTTCCTCGTGCTTTACGGTATCGCCGC
TCCCGATTCGCAGCGCATCGCCTTCTATCGCCTTCTTGACGAGTTCTTCTGAGCGACGATGAACATCAAAAAGTTTGCAAAA
CAAGCAACAGTATTAACCTTTACTACCGCACTGCTGGCAGGAGGCGCAACTCAAGCGTTTGCGAAAGAAACGAACCAAAAG
CCATATAAGGAAACATACGGCATTTCCCATATTACACGCCATGATATGCTGCAAATCCCTGAACAGCAAAAAAATGAAAAAT
ATCAAGTTTCTGAATTTGATTCGTCCACAATTAAAAATATCTCTTCTGCAAAAGGCCTGGACGTTTGGGACAGCTGGCCATT
ACAAAACGCTGACGGCACTGTCGCAAACTATCACGGCTACCACATCGTCTTTGCATTAGCCGGAGATCCTAAAAATGCGGA
TGACACATCGATTTACATGTTCTATCAAAAAGTCGGCGAAACTTCTATTGACAGCTGGAAAAACGCTGGCCGCGTCTTTAAA
GACAGCGACAAATTCGATGCAAATGATTCTATCCTAAAAGACCAAACACAAGAATGGTCAGGTTCAGCCACATTTACATCT
GACGGAAAAATCCGTTTATTCTACACTGATTTCTCCGGTAAACATTACGGCAAACAAACACTGACAACTGCACAAGTTAACG
TATCAGCATCAGACAGCTCTTTGAACATCAACGGTGTAGAGGATTATAAATCAATCTTTGACGGTGACGGAAAAACGTATC
AAAATGTACAGCAGTTCATCGATGAAGGCAACTACAGCTCAGGCGACAACCATACGCTGAGAGATCCTCACTACGTAGAAG
ATAAAGGCCACAAATACTTAGTATTTGAAGCAAACACTGGAACTGAAGATGGCTACCAAGGCGAAGAATCTTTATTTAACA
AAGCATACTATGGCAAAAGCACATCATTCTTCCGTCAAGAAAGTCAAAAACTTCTGCAAAGCGATAAAAAACGCACGGCTG
AGTTAGCAAACGGCGCTCTCGGTATGATTGAGCTAAACGATGATTACACACTGAAAAAAGTGATGAAACCGCTGATTGCAT
CTAACACAGTAACAGATGAAATTGAACGCGCGAACGTCTTTAAAATGAACGGCAAATGGTACCTGTTCACTGACTCCCGCG
GATCAAAAATGACGATTGACGGCATTACGTCTAACGATATTTACATGCTTGGTTATGTTTCTAATTCTTTAACTGGCCCATAC
AAGCCGCTGAACAAAACTGGCCTTGTGTTAAAAATGGATCTTGATCCTAACGATGTAACCTTTACTTACTCACACTTCGCTGT
ACCTCAAGCGAAAGGAAACAATGTCGTGATTACAAGCTATATGACAAACAGAGGATTCTACGCAGACAAACAATCAACGTT
TGCGCCGAGCTTCCTGCTGAACATCAAAGGCAAGAAAACATCTGTTGTCAAAGACAGCATCCTTGAACAAGGACAATTAAC
AGTTAACAAATAATCAGACCCCGTAGAAAAGATCAAAGGATCTTC
tLC-003 (pLC017) Upstream homologous arm, genome edit, downstream homologous arm (SEQ ID NO: 89)
TTGAGATCCTTTTTTTCTGCGCGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCTCACACAGGAAACAGCTATGACATGATTACGAATTCAGTGTCGGCGGCGG
CTTTCTTGCCGACGTAACCTTGGCCACGGCGGTAGACCTTGAAGCGGGCACGCATGCTGGGCAGGTGCTCTTCAAATTCAT
CTTCGACGGTGTTCGGTGGCAGGCGGTCGAGGCGCACTTCACCGGTTTGGCTAACCCACAGCAAATGGTCGTCAAGGCTGT
CCTTGCGCACGGCGAACAGCTGGGCCAATTGGTCGATAGTCGGATTGTTGTTCAAGTTCATCATAAAGCCCCCATTACCCAT
TCATAGGTGATTTTCACAGTTGGCGCTATCACGGTGTAGCGCACTACCAAGGCTGCTACAGCAGCATCGCAACAGGGGCTT
TCTCACGCTGCCTTTTGGACAGTTTCCCTCTCCACGGACGGCCTGCGTTACCGCGCAGGCCGTCTGGAACACCCTTGCCACC
GCTCCCGATCGGGGAGACGGCTTCATCATGCCCAAGCGCGATGAACGGCGTCAACCATTITGTAGTGAATATTTTTGCTCAC
TACATTCTGTCGTTTTGTCCGACAATCAACGCGAGCGTTAGGATCCGACGTCCATAAAAAAATGGCGCCGATGGGCGCCAT
TTTTCACTGTTACAGGGTGGCGGTCTGCTGGGTCTTTTTGGTTTCCGGTTCCCACAGCGCTTCCAGTTCTTCCAAGGTCTTAC
CTTTGGTTTCGGGCACGAACTTCCACATGAACAGGGCGGCCAGCACGCCCATGCAGCCGTAGATCCAGTAGCTGAAGCCGT
TGTGGAAGTGCGCCACCAACCAGCTGTTCTTGTCCATCATTGGGAAGGTCCAGGACACGAAGTAGTTGGCCAGCCACTGGG
CGGCCACGGCAATGGCCAGGGCTTTGCCACGGATGGCATTGGGGAAGATTTCGGACAGCAGGACCCAGCACACGGGACC
CCACGACATGGCGAAGGCGGCCACGTAGAACAACATCGACAGCAGGGCGACAATGCCCGGGGCCTGGGTGTAGAACGCC
GTGCCCAAGCTGAACATGCCGATGGCCATGCCCAGGGCGCCGATGATTTGCAGCGGCTTACGGCCGAATTTATCCACGGTC
ATGATCGCCAGGACCGTAAACGTCAAGTTAATCACACCCACGATAATGGTCTGCAGCAGGGCGATGTCGGTCGAGGCGCC
CAGGGTCTTAAACACTTCTGGCGCGTAATACAACACCACGTTGATCCCGACGAACTGCTGGAAAATGGACAACATCACGCC
AATCACGATCACCCCCACGCCGAACATCAGCAAGCGCCCGCCGGTCTTGCGGCCGTGGTCCAAGGAGTGTTTGATCTCTTG
CACGGCCTGCGTGGCCAGGGTATTGCCCATGATCTTCCGCAAGATGCCTTCGGCTTGTTCCTGCTTGCCACGGCTCATCAGC
CAGCGCGGGCTCTCGGGGACGGTGTAAGCTTGGCACTGGCCGTCGTTTTACAACGTCGTGACCGGAATTGCCAGCTGGGG
CGCCCTCTGGTAAGGTTGGGAAGCCCTGCAAACAGGATGAGGATCGTTTCGCATGATTGAACAAGATGGATTGCACGCAG
GTTCTCCGGCCGCTTGGGTGGAGAGGCTATTCGGCTATGACTGGGCACAACAGACAATCGGCTGCTCTGATGCCGCCGTGT
TCCGGCTGTCAGCGCAGGGGCGCCCGGTTCTTTTTGTCAAGACCGACCTGTCCGGTGCCCTGAATGAACTCCAAGACGAGG
CAGCGCGGCTATCGTGGCTGGCCACGACGGGCGTTCCTTGCGCAGCTGTGCTCGACGTTGTCACTGAAGCGGGAAGGGAC
TGGCTGCTATTGGGCGAAGTGCCGGGGCAGGATCTCCTGTCATCTCACCTTGCTCCTGCCGAGAAAGTATCCATCATGGCT
GATGCAATGCGGCGGCTGCATACGCTTGATCCGGCTACCTGCCCATTCGACCACCAAGCGAAACATCGCATCGAGCGAGCA
CGTACTCGGATGGAAGCCGGTCTTGTCGATCAGGATGATCTGGACGAAGAGCATCAGGGGCTCGCGCCAGCCGAACTGTT
CGCCAGGCTCAAGGCGCGGATGCCCGACGGCGAGGATCTCGTCGTGACCCATGGCGATGCCTGCTTGCCGAATATCATGG
TGGAAAATGGCCGCTTTTCTGGATTCATCGACTGTGGCCGGCTGGGTGTGGCGGACCGCTATCAGGACATAGCGTTGGCTA
CCCGTGATATTGCTGAAGAGCTTGGCGGCGAATGGGCTGACCGCTTCCTCGTGCTTTACGGTATCGCCGCTCCCGATTCGCA
GCGCATCGCCTTCTATCGCCTTCTTGACGAGTTCTTCTGAGCGACGATGAACATCAAAAAGTTTGCAAAACAAGCAACAGTA
TTAACCTTTACTACCGCACTGCTGGCAGGAGGCGCAACTCAAGCGTTTGCGAAAGAAACGAACCAAAAGCCATATAAGGAA
ACATACGGCATTTCCCATATTACACGCCATGATATGCTGCAAATCCCTGAACAGCAAAAAAATGAAAAATATCAAGTTTCTG
AATTTGATTCGTCCACAATTAAAAATATCTCTTCTGCAAAAGGCCTGGACGTTTGGGACAGCTGGCCATTACAAAACGCTGA
CGGCACTGTCGCAAACTATCACGGCTACCACATCGTCTTTGCATTAGCCGGAGATCCTAAAAATGCGGATGACACATCGATT
TACATGTTCTATCAAAAAGTCGGCGAAACTTCTATTGACAGCTGGAAAAACGCTGGCCGCGTCTTTAAAGACAGCGACAAA
TTCGATGCAAATGATTCTATCCTAAAAGACCAAACACAAGAATGGTCAGGTTCAGCCACATTTACATCTGACGGAAAAATCC
GTTTATTCTACACTGATTTCTCCGGTAAACATTACGGCAAACAAACACTGACAACTGCACAAGTTAACGTATCAGCATCAGA
CAGCTCTTTGAACATCAACGGTGTAGAGGATTATAAATCAATCTTTGACGGTGACGGAAAAACGTATCAAAATGTACAGCA
GTTCATCGATGAAGGCAACTACAGCTCAGGCGACAACCATACGCTGAGAGATCCTCACTACGTAGAAGATAAAGGCCACAA
ATACTTAGTATTTGAAGCAAACACTGGAACTGAAGATGGCTACCAAGGCGAAGAATCTTTATTTAACAAAGCATACTATGG
CAAAAGCACATCATTCTTCCGTCAAGAAAGTCAAAAACTTCTGCAAAGCGATAAAAAACGCACGGCTGAGTTAGCAAACGG
CGCTCTCGGTATGATTGAGCTAAACGATGATTACACACTGAAAAAAGTGATGAAACCGCTGATTGCATCTAACACAGTAAC
AGATGAAATTGAACGCGCGAACGTCTTTAAAATGAACGGCAAATGGTACCTGTTCACTGACTCCCGCGGATCAAAAATGAC
GATTGACGGCATTACGTCTAACGATATTTACATGCTTGGTTATGTTTCTAATTCTTTAACTGGCCCATACAAGCCGCTGAACA
AAACTGGCCTTGTGTTAAAAATGGATCTTGATCCTAACGATGTAACCTTTACTTACTCACACTTCGCTGTACCTCAAGCGAAA
GGAAACAATGTCGTGATTACAAGCTATATGACAAACAGAGGATTCTACGCAGACAAACAATCAACGTTTGCGCCGAGCTTC
CTGCTGAACATCAAAGGCAAGAAAACATCTGTTGTCAAAGACAGCATCCTTGAACAAGGACAATTAACAGTTAACAAATAA
TCAGACCCCGTAGAAAAGATCAAAGGATCTTC
tLC008 (pLC088) Upstream homologous arm, downstream homologous arm (SEQ ID NO: 90)
TTGAGATCCTTTTTTTCTGCGCGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCTCACACAGGAAACAGCTATGACATGATTACGAATTCAACCCCAGCAGTCGC
TGGAGGCTGCCGCGGTCAAAGCCCGTCAGGCCGGTTTCAGCCCGCTGATTCTCGGCGACCTGGAAGGTGAATCGCGCGAA
GTGGCCAAGGTGCATGCCGGTATCGCCCGGCAAATCGTTCAACACGGCCAGCCGCTCAAAGCGCCCTGCGTGATCCTGTCG
GGTGGCGAAACCACCGTGACCGTGCGCGGCAATGGCCGTGGCGGGCGTAACGCCGAGTTCCTGCTCAGCCTCACCGAAAG
CCTGAAAGGCCTGCCGGGCGTGTACGCCCTGGCCGGTGACACCGACGGCATCGATGGCTCGGAAGAAAACGCCGGTGCCT
TCATGACCCCGGCCAGCTACGCCAGCGCCGAAGCCTTGGGCCTGTCGGCCAGCGACGAGCTGGACAACAACAACGGCTAC
GGCTACTTCGCCGCGCTGGATGCGCTGATCGTCACCGAACCGACCCGCACCAACGTCAACGACTTCCGCGCCATCCTGATCC
TTGAGACTGCCCAATCATGAGCACACCGTCAACCATTGAGCCTTGTGGGGCGGATCCCCCTCAAGTCAAAAGCCTCCGGTC
GGAGGCTTTTGACTTTCTGCTATGGAGGTCAGGCCTAGGTGATTATTGTTTCTCGCAAAAAACTTCAAAATAAACGCTTGAC
AATTAATCATCGGCTCGTATAATGTGTGGAATTGTGAGCGGATAACAATTCCCGGGACTATTCTCGGCCTCCAGCGCCGAG
GCGAGGCTCAACGTTACCGCGCAGGCCGCCGGATGGCGCCGCGCCCAAGTACATTGTGGGGATACATGCAGACACTAAAG
GTCGACCTGGGCGAGCGTAGCTACCCGATCTACATTGGCGAAGGCCTGCTGGACCAGCCTGAGCTGCTGGCGCCGCACAT
TGCGGGTCGGCAGGTTGCCATCGTCTCCAACGAGACCGTCGCGCCGCTGTATCTCGAACGCCTGAGCAAGGCCCTTGGTGC
CTACTCGGTACTGCCCGTTGTGTTGCCCGATGGTGAAGCCCACAAGAACTGGGAAACATTGCAGCTGATCTTCGATGGCCT
GCTGACAGCTCGGCATGATCGTCGTACTACTGTGGTCGCTCTGGGTGGTGGCGTGATTGGTGACATGGCTGGTTTCGCAGC
CGCCTGCTACCAGCGCGGTGTCGATTTCATCCAGGTGCCGACTACCCTGTTGTCCCAGGTCGACTCTTCCGTGGGTGGCAAG
ACCGGCATCAATCATCCGCTGGGCAAGAACATGGTCGGTGCGTTCTATCAGCCCAAGGCGGTGCTGATCGATACCACCAGC
CTCAAGACCCTGCCAGCGCGCGAGCTGTCTGCAGGTCTGGCCGAAGTGATCAAGTACGGCCTGATTTGCGACAAGCCGTTC
CTCGCCTGGCTCGAAGACAATATGCAGGCACTAAAGCTTGGCACTGGCCGTCGTTTTACAACGTCGTGACCGGAATTGCCA
GCTGGGGCGCCCTCTGGTAAGGTTGGGAAGCCCTGCAAACAGGATGAGGATCGTTTCGCATGATTGAACAAGATGGATTG
CACGCAGGTTCTCCGGCCGCTTGGGTGGAGAGGCTATTCGGCTATGACTGGGCACAACAGACAATCGGCTGCTCTGATGCC
GCCGTGTTCCGGCTGTCAGCGCAGGGGCGCCCGGTTCTTTTTGTCAAGACCGACCTGTCCGGTGCCCTGAATGAACTCCAA
GACGAGGCAGCGCGGCTATCGTGGCTGGCCACGACGGGCGTTCCTTGCGCAGCTGTGCTCGACGTTGTCACTGAAGCGGG
AAGGGACTGGCTGCTATTGGGCGAAGTGCCGGGGCAGGATCTCCTGTCATCTCACCTTGCTCCTGCCGAGAAAGTATCCAT
CATGGCTGATGCAATGCGGCGGCTGCATACGCTTGATCCGGCTACCTGCCCATTCGACCACCAAGCGAAACATCGCATCGA
GCGAGCACGTACTCGGATGGAAGCCGGTCTTGTCGATCAGGATGATCTGGACGAAGAGCATCAGGGGCTCGCGCCAGCCG
AACTGTTCGCCAGGCTCAAGGCGCGGATGCCCGACGGCGAGGATCTCGTCGTGACCCATGGCGATGCCTGCTTGCCGAAT
ATCATGGTGGAAAATGGCCGCTTTTCTGGATTCATCGACTGTGGCCGGCTGGGTGTGGCGGACCGCTATCAGGACATAGC
GTTGGCTACCCGTGATATTGCTGAAGAGCTTGGCGGCGAATGGGCTGACCGCTTCCTCGTGCTTTACGGTATCGCCGCTCCC
GATTCGCAGCGCATCGCCTTCTATCGCCTTCTTGACGAGTTCTTCTGAGCGACGATGAACATCAAAAAGTTTGCAAAACAAG
CAACAGTATTAACCTTTACTACCGCACTGCTGGCAGGAGGCGCAACTCAAGCGTTTGCGAAAGAAACGAACCAAAAGCCAT
ATAAGGAAACATACGGCATTTCCCATATTACACGCCATGATATGCTGCAAATCCCTGAACAGCAAAAAAATGAAAAATATCA
AGTTTCTGAATTTGATTCGTCCACAATTAAAAATATCTCTTCTGCAAAAGGCCTGGACGTTTGGGACAGCTGGCCATTACAA
AACGCTGACGGCACTGTCGCAAACTATCACGGCTACCACATCGTCTTTGCATTAGCCGGAGATCCTAAAAATGCGGATGAC
ACATCGATTTACATGTTCTATCAAAAAGTCGGCGAAACTTCTATTGACAGCTGGAAAAACGCTGGCCGCGTCTTTAAAGACA
GCGACAAATTCGATGCAAATGATTCTATCCTAAAAGACCAAACACAAGAATGGTCAGGTTCAGCCACATTTACATCTGACG
GAAAAATCCGTTTATTCTACACTGATTTCTCCGGTAAACATTACGGCAAACAAACACTGACAACTGCACAAGTTAACGTATC
AGCATCAGACAGCTCTTTGAACATCAACGGTGTAGAGGATTATAAATCAATCTTTGACGGTGACGGAAAAACGTATCAAAA
TGTACAGCAGTTCATCGATGAAGGCAACTACAGCTCAGGCGACAACCATACGCTGAGAGATCCTCACTACGTAGAAGATAA
AGGCCACAAATACTTAGTATTTGAAGCAAACACTGGAACTGAAGATGGCTACCAAGGCGAAGAATCTTTATTTAACAAAGC
ATACTATGGCAAAAGCACATCATTCTTCCGTCAAGAAAGTCAAAAACTTCTGCAAAGCGATAAAAAACGCACGGCTGAGTT
AGCAAACGGCGCTCTCGGTATGATTGAGCTAAACGATGATTACACACTGAAAAAAGTGATGAAACCGCTGATTGCATCTAA
CACAGTAACAGATGAAATTGAACGCGCGAACGTCTTTAAAATGAACGGCAAATGGTACCTGTTCACTGACTCCCGCGGATC
AAAAATGACGATTGACGGCATTACGTCTAACGATATTTACATGCTTGGTTATGTTTCTAATTCTTTAACTGGCCCATACAAGC
CGCTGAACAAAACTGGCCTTGTGTTAAAAATGGATCTTGATCCTAACGATGTAACCTTTACTTACTCACACTTCGCTGTACCT
CAAGCGAAAGGAAACAATGTCGTGATTACAAGCTATATGACAAACAGAGGATTCTACGCAGACAAACAATCAACGTTTGCG
CCGAGCTTCCTGCTGAACATCAAAGGCAAGAAAACATCTGTTGTCAAAGACAGCATCCTTGAACAAGGACAATTAACAGTT
AACAAATAATCAGACCCCGTAGAAACGGCCAGCCTCGCAGAGCAGGATTCCCGTTGAGCACCGCCAGGTGCGAATAAGGG
ACAGTGAAGAAGGAACACCCGCTCGCGGGTGGGCCTACTTCACCTATCCTGCCCGGCTGACGCCGTTGGATACACCAAGG
AAAGTCTACACGAACCCTTTGGCAAAATCCTGTATATCGTGCGAAAAAGGATGGATATACCGAAAAAATCGCTATAATGAC
CCCGAAGCAGGGTTATGCAGCGGAAAAGAGATCAAAGGATCTTC
pLC028 Upstream homologous arm, [symbol] , downstream homologous arm, start codon
of PP 4302
(SEQ ID NO: 91)
TTGAGATCCTTTTTTTCTGCGCGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCTGGCCTTTTGCTCACA
TGTTCTTTCCTGCGTTATCCCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCCAATACGCAAACCGCCTCTCCCCGCGCGTTGGC
CGATTCATTAATGCAGCTGGCACGACAGGTTTCCCGACTGGAAAGCGGGCAGTGAGCGCAACGCAATTAATGTGAGTTAG
GAATTCGCCACGGTCTACACCTATGCCATTTCCGATGTACCGGGCGACCTCGCCACGGTAATCGCCTCCGGCCCCACCGTGG
CCGACCCGAGCACCTCGGCCGACGCCCTGGCCATCCTCAAACGCTACAACATCGAAGCGCCCAAAGCGGTCATCGACTGGC
TGAACAACCCGGCCTCGGAAACCGTCAAGGCCGATGACCCGGCCCTGGCCCGCAGCCACTTCCAGTTGATCGCCAAACCCC
AGCAGTCGCTGGAGGCTGCCGCGGTCAAAGCCCGTCAGGCCGGTTTCAGCCCGCTGATTCTCGGCGACCTGGAAGGTGAA
TCGCGCGAAGTGGCCAAGGTGCATGCCGGTATCGCCCGGCAAATCGTTCAACACGGCCAGCCGCTCAAAGCGCCCTGCGT
GATCCTGTCGGGTGGCGAAACCACCGTGACCGTGCGCGGCAATGGCCGTGGCGGGCGTAACGCCGAGTTCCTGCTCAGCC
TCACCGAAAGCCTGAAAGGCCTGCCGGGCGTGTACGCCCTGGCCGGTGACACCGACGGCATCGATGGCTCGGAAGAAAAC
GCCGGTGCCTTCATGACCCCGGCCAGCTACGCCAGCGCCGAAGCCTTGGGCCTGTCGGCCAGCGACGAGCTGGACAACAA
CAACGGCTACGGCTACTTCGCCGCGCTGGATGCGCTGATCGTCACCGAACCGACCCGCACCAACGTCAACGACTTCCGCGC
CATCCTGATCCTTGAGACTGCCCAATCATGAGCACACCGTCAACCATTGAGCCTTGTGGGGCGGATCCCCCTCAAGTCAAAA
GCCTCCGGTCGGAGGCTTTTGACTTTCTGCTATGGAGGTCAGGCCTAGGTGATTATTGTTTCTCGCAAAAAACTTCAAAATA
AACGCTTTACACTTTATGCTTCCGGCTCGTATGTTGTGGTGATCAGACCTGGAATTGTGAGCGGATAACAATTGGTACTTTA
AAAATAGAAAATAGCAAGGAGATATACATATGTCACACCCCGCGTTAACGCAACTGCGTGCGCTGCGCTATTTTAAAGAGA
TCCCTGCCCTGGATCCGCAACTGCTCGACTGGCTGTTGCTGCAGGATTCCGTGACAAAACGTTTTGAACAGCAGGGAAAAA
CGGTAAGCGTGACGATGATCCGCGAAGGGTTTGTCGAGCAGAATGAAATCCCCGAAGAACTGCCGCTGCTGCCGAAAGAG
TCTCGTTACTGGTTACGTGAAATTTTGTTATGTGCCGATGGTGAACCGTGGCTTGCCGGTCGTACCGTCGTTCCTGTGTCAA
CGTTAAGCGGGCCGGAGCTGGCGTTACAAAAATTGGGTAAAACGCCGTTAGGACGCTATCTGTTCACATCATCGACATTAA
CCCGGGACTTTATTGAGATAGGCCGTGATGCCGGGCTGTGGGGGCGACGTTCCCGCCTGCGATTAAGCGGTAAACCGCTG
TTGCTAACAGAACTGTTTTTACCGGCGTCACCGTTGTACTAAGATATGGATCCATCAATAAAACGAAAGGCTCAGTCGAAAG
ACTGGGCCTTTCGTTTTATCTGTTGGCCTTGTGTTGCTCCCAT [symbol] [symbol] [symbol] [symbol] [symbol]
TCGCGATTGGAGGGC
AAAGCCCTCCCTGAATACCGCTCTACCCGACCGCGGAAAACCGAGGCCCAAAGAGGCCCACCGCTCCCCCATCAACCTCCA
CTGACTGCCCCATGTACACCAAAAATTTCGTCAACCCGTGCCCCGACTGGGCCACGGCGTTACTCAACGGCTTCAGCCAGGT
GCTGCTGCTGCGCAACCCCCTGTGCGGCCTGTGCTGCCTGCTGGCCATCCTGCTGACCGCACCCAACCTGGTCGGCGGCGC
GCTGCTTGGCGCCCTGGCCGGCCTGCTCACCGCCCAGGCACGCGGCTATGACCGTGCCGACCGCCAGGCCGGGCTTTACTG
CTACAACGGCGTACTGATCGGCATCCTGATCAGCGCCGTGCTGCCCTGGTCGGCCATATTGCCCCCCCTGATCATCGCCGCC
GGCGGCTTGTCCAGCATCATCACGCACCAGTGGCGCAAGCGTGGCGGCAAGTTGCTGATTGCCTACACCTCCCCTTTCGTAT
TGCTGGGCTGGGCCACACTGCTGGTCGCCAGCCCCTCGCCCAGCGGGTTCGTCGAAGCCGACCCGCTGTATGCGTTGGCCC
GCGGCGTGGGCCAGATTTTCCTGCTCGACCAGCCCTTGGCCGGGTTGCTGATCATCGTCGGCATGGGATCCCTAGCTTCAC
GCTGCCGCAAGCACTCAGGGCGCAAGGGCTGCTAAAGGAAGCGGAACACGTAGAAAGCCAGTCCGCAGAAACGGTGCTG
ACCCCGGATGAATGTCAGCTACTGGGCTATCTGGACAAGGGAAAACGCAAGCGCAAAGAGAAAGCAGGTAGCTTGCAGT
GGGCTTACATGGCGATAGCTAGACTGGGCGGTTTTATGGACAGCAAGCGAACCGGAATTGCCAGCTGGGGCGCCCTCTGG
TAAGGTTGGGAAGCCCTGCAAAGTAAACTGGATGGCTTTCTTGCCGCCAAGGATCTGATGGCGCAGGGGATCAAGATCTG
ATCAAGAGACAGGATGAGGATCGTTTCGCATGATTGAACAAGATGGATTGCACGCAGGTTCTCCGGCCGCTTGGGTGGAG
AGGCTATTCGGCTATGACTGGGCACAACAGACAATCGGCTGCTCTGATGCCGCCGTGTTCCGGCTGTCAGCGCAGGGGCG
CCCGGTTCTTTTTGTCAAGACCGACCTGTCCGGTGCCCTGAATGAACTCCAAGACGAGGCAGCGCGGCTATCGTGGCTGGC
CACGACGGGCGTTCCTTGCGCAGCTGTGCTCGACGTTGTCACTGAAGCGGGAAGGGACTGGCTGCTATTGGGCGAAGTGC
CGGGGCAGGATCTCCTGTCATCTCACCTTGCTCCTGCCGAGAAAGTATCCATCATGGCTGATGCAATGCGGCGGCTGCATA
CGCTTGATCCGGCTACCTGCCCATTCGACCACCAAGCGAAACATCGCATCGAGCGAGCACGTACTCGGATGGAAGCCGGTC
TTGTCGATCAGGATGATCTGGACGAAGAGCATCAGGGGCTCGCGCCAGCCGAACTGTTCGCCAGGCTCAAGGCGCGGATG
CCCGACGGCGAGGATCTCGTCGTGACCCATGGCGATGCCTGCTTGCCGAATATCATGGTGGAAAATGGCCGCTTTTCTGGA
TTCATCGACTGTGGCCGGCTGGGTGTGGCGGACCGCTATCAGGACATAGCGTTGGCTACCCGTGATATTGCTGAAGAGCTT
GGCGGCGAATGGGCTGACCGCTTCCTCGTGCTTTACGGTATCGCCGCTCCCGATTCGCAGCGCATCGCCTTCTATCGCCTTC
TTGACGAGTTCTTCTGAGCGGGACTCTGGGGTTCGCTAGAGGATCGATCCTTTTTAACCCATCACATATACCTGCCGTTCACT
ATTATTTAGTGAAATGAGATATTATGATATTTTCTGAATTGTGATTAAAAAGGCAACTTTATGCCCATGCAACAGAAACTATA
AAAAATACAGAGAATGAAAAGAAACAGATAGATTTTTTAGTTCTTTAGGCCCGTAGTCTGCAAATCCTITTATGATTTTCTAT
CAAACAAAAGAGGAAAATAGACCAGTTGCAATCCAAACGAGAGTCTAATAGAATGAGGTCGAAAAGTAAATCGCGCGGGT
TTGTTACTGATAAAGCAGGCAAGACCTAAAATGTGTAAAGGGCAAAGTGTATACTTTGGCGTCACCCCTTACATATTTTAGG
TCTTTTTTTATTGTGCGTAACTAACTTGCCATCTTCAAACAGGAGGGCTGGAAGAAGCAGACCGCTAACACAGTACATAAAA
AAGGAGACATGAACGATGAACATCAAAAAGTTTGCAAAACAAGCAACAGTATTAACCTTTACTACCGCACTGCTGGCAGGA
GGCGCAACTCAAGCGTTTGCGAAAGAAACGAACCAAAAGCCATATAAGGAAACATACGGCATTTCCCATATTACACGCCAT
GATATGCTGCAAATCCCTGAACAGCAAAAAAATGAAAAATATCAAGTTTCTGAATTTGATTCGTCCACAATTAAAAATATCT
CTTCTGCAAAAGGCCTGGACGTTTGGGACAGCTGGCCATTACAAAACGCTGACGGCACTGTCGCAAACTATCACGGCTACC
ACATCGTCTTTGCATTAGCCGGAGATCCTAAAAATGCGGATGACACATCGATTTACATGTTCTATCAAAAAGTCGGCGAAAC
TTCTATTGACAGCTGGAAAAACGCTGGCCGCGTCTTTAAAGACAGCGACAAATTCGATGCAAATGATTCTATCCTAAAAGAC
CAAACACAAGAATGGTCAGGTTCAGCCACATTTACATCTGACGGAAAAATCCGTTTATTCTACACTGATTTCTCCGGTAAAC
ATTACGGCAAACAAACACTGACAACTGCACAAGTTAACGTATCAGCATCAGACAGCTCTTTGAACATCAACGGTGTAGAGG
ATTATAAATCAATCTTTGACGGTGACGGAAAAACGTATCAAAATGTACAGCAGTTCATCGATGAAGGCAACTACAGCTCAG
GCGACAACCATACGCTGAGAGATCCTCACTACGTAGAAGATAAAGGCCACAAATACTTAGTATTTGAAGCAAACACTGGAA
CTGAAGATGGCTACCAAGGCGAAGAATCTTTATTTAACAAAGCATACTATGGCAAAAGCACATCATTCTTCCGTCAAGAAA
GTCAAAAACTTCTGCAAAGCGATAAAAAACGCACGGCTGAGTTAGCAAACGGCGCTCTCGGTATGATTGAGCTAAACGAT
GATTACACACTGAAAAAAGTGATGAAACCGCTGATTGCATCTAACACAGTAACAGATGAAATTGAACGCGCGAACGTCTTT
AAAATGAACGGCAAATGGTACCTGTTCACTGACTCCCGCGGATCAAAAATGACGATTGACGGCATTACGTCTAACGATATT
TACATGCTTGGTTATGTTTCTAATTCTTTAACTGGCCCATACAAGCCGCTGAACAAAACTGGCCTTGTGTTAAAAATGGATCT
TGATCCTAACGATGTAACCTTTACTTACTCACACTTCGCTGTACCTCAAGCGAAAGGAAACAATGTCGTGATTACAAGCTAT
ATGACAAACAGAGGATTCTACGCAGACAAACAATCAACGTTTGCGCCGAGCTTCCTGCTGAACATCAAAGGCAAGAAAACA
TCTGTTGTCAAAGACAGCATCCTTGAACAAGGACAATTAACAGTTAACAAATAAAAACGCAAAAGAAAATGCCGATGGGTA
CCGAGCGAAATGACCGACCAAGCGACGCCCAACCTGCCATCACGAGATTTCGATTCCACCGCCGCCTTCTATGAAAGGTTG
GGCTTCGGAATCGTTTTCCGGGACGCCCTCGCGGACGTGCTCATAGTCCACGACGCCCGTGATTTTGTAGCCCTGGCCGAC
GGCCAGCAGGTAGGCCGACAGGCTCATGCCGGCCGCCGCCGCCTTTTCCTCAATCGCTCTTCGTTCGTCTGGAAGGCAGTA
CACCTTGATAGGTGGGCTGCCCTTCCTGGTTGGCTTGGTTTCATCAGCCATCCGCTTGCCCTCATCTGTTACGCCGGCGGTA
GCCGGCCAGCCTCGCAGAGCAGGATTCCCGTTGAGCACCGCCAGGTGCGAATAAGGGACAGTGAAGAAGGAACACCCGC
TCGCGGGTGGGCCTACTTCACCTATCCTGCCCGGCTGACGCCGTTGGATACACCAAGGAAAGTCTACACGAACCCTTTGGC
AAAATCCTGTATATCGTGCGAAAAAGGATGGATATACCGAAAAAATCGCTATAATGACCCCGAAGCAGGGTTATGCAGCG
GAAAAGCGCTGCTTCCCTGCTGTTTTGTGGAATATCTACCGACTGGAAACAGGCAAATGCAGGAAATTACTGAACTGAGGG
GACAGGCGAGAGACGATGCCAAAGAGCTCCTGAAAATCTCGATAACTCAAAAAATACGCCCGGTAGTGATCTTATTTCATT
ATGGTGAAAGTTGGAACCTCTTACGTGCCGATCAACGTCTCATTTTCGCCAAAAGTTGGCCCAGGGCTTCCCGGTATCAACA
GGGACACCAGGATTTATTTATTCTGCGAAGTGATCTTCCGTCACAGGTATTTATTCGGCGCAAAGTGCGTCGGGTGATGCT
GCCAACTTACTGATTTAGTGTATGATGGTGTTTTTGAGGTGCTCCAGTGGCTTCTGTTTCTATCAGCTCCTGAAAATCTCGAT
AACTCAAAAAATACGCCCGGTAGTGATCTTATTTCATTATGGTGAAAGTTGGAACCTCTTACGTGCCGATCAACGTCTCATTT
TCGCCAAAAGTTGGCCCAGGGCTTCCCGGTATCAACAGGGACACCAGGATTTATTTATTCTGCGAAGTGATCTTCCGTCACA
GGTATTTATTCGGCGCAAAGTGCGTCGGGTGATGCTGCCAACTTACTGATTTAGTGTATGATGGTGTTTTTGAGGTGCTCCA
GTGGCTTCTGTTTCTATCAGGGCTGGATGATCCTCCAGCGCGGGGATCTCATGCTGGAGTTCTTCGCCCACCCCAAAAGGAT
CTAGGTGAAGATCCTTTTTGATAATCTCATGACCAAAATCCCTTAACGTGAGTTTTCGTTCCACTGAGCGTCAGACCCCGTAG
AAAAGATCAAAGGATCTTC

While a number of exemplary aspects and embodiments have been discussed above, those of skill in the art will recognize certain modifications, permutations, additions and sub combinations thereof. It is therefore intended that the following appended claims and claims hereafter introduced are interpreted to include all such modifications, permutations, additions and sub-combinations as are within their true spirit and scope.

What is claimed is:

1. A genetically modified microorganism comprising at least one exogenous gene addition, wherein the at least one added gene encodes at least one of a decarboxylase, a dehydratase, or a monooxygenase.

2. The genetically modified microorganism of claim 1 that produces 33.7 g/L muconate at 0.18 g/L/h at a 46% molar yield.

3. The genetically modified microorganism of claim 2 wherein the yield of the muconate produced is up to 92% of the maximum theoretical yield.

4. A process for producing muconic acid, the process comprising contacting a culture broth containing lignin depolymerization compounds with the microorganism of claim 1.

5. A process for producing adipic acid, the process comprising:
separating muconic acid from a culture broth comprising muconic acid, impurities, and a microorganism;
purifying the separated muconic acid; and
hydrogenating at least a portion of the purified muconic acid to produce the adipic acid.


SEQUENCE LISTING (91 sequences)

SEQ ID NO: 1 - DNA, 32 bp, synthetic construct
  source 1..32: mol_type = other DNA
gcgacacgaagctgtatagccctgccctattg

SEQ ID NO: 2 - DNA, 28 bp, synthetic construct
  source 1..28: mol_type = other DNA
gctatacagcttcgtgtcgctcaaggcg

SEQ ID NO: 3 - DNA, 33 bp, synthetic construct
  source 1..33: mol_type = other DNA
acctcgtattctctgaaattgttatccgctcac

SEQ ID NO: 4 - DNA, 30 bp, synthetic construct
  source 1..30: mol_type = other DNA
aatttcacacaatacgaggtaagcacgatg

SEQ ID NO: 5 - DNA, 28 bp, synthetic construct
  source 1..28: mol_type = other DNA
ccgcggccgccatcattgagaccgcgcg

SEQ ID NO: 6 - DNA, 31 bp, synthetic construct
  source 1..31: mol_type = other DNA
ccgcggccgcgtgacataacctcgaactcag

SEQ ID NO: 7 - DNA, 28 bp, synthetic construct
  source 1..28: mol_type = other DNA
caggacatcatcagccctcctgcaacgc

SEQ ID NO: 8 - DNA, 28 bp, synthetic construct
  source 1..28: mol_type = other DNA
ggagggctgatgatgtcctgcgcaagcc

SEQ ID NO: 9 - DNA, 29 bp, synthetic construct
  source 1..29: mol_type = other DNA
aacctcgaactcagatgcgcttgaacagg

SEQ ID NO: 10 - DNA, 37 bp, synthetic construct
  source 1..37: mol_type = other DNA
gcgcatctgagttcgaggttatgtcactgtgattttg

SEQ ID NO: 11 - DNA, 50 bp, synthetic construct
  source 1..50: mol_type = other DNA
atccccgggtaccgagctcgaattcatgaccgtgaaaatttcccacactg

SEQ ID NO: 12 - DNA, 43 bp, synthetic construct
  source 1..43: mol_type = other DNA
cagctatgaccatgattacgaattcttgaatgccggcaacccg

SEQ ID NO: 13 - DNA, 39 bp, synthetic construct
  source 1..39: mol_type = other DNA
ccgaaaaagtgccacctgacgtcggccttgctgctgcag

SEQ ID NO: 14 - DNA, 37 bp, synthetic construct
  source 1..37: mol_type = other DNA
gccgcagctcgagatctggaattgtgagaacgcctgg

SEQ ID NO: 15 - DNA, 37 bp, synthetic construct
  source 1..37: mol_type = other DNA
agatctcgagctgcgggccgcggtgaagcttggggcc

SEQ ID NO: 16 - DNA, 40 bp, synthetic construct
  source 1..40: mol_type = other DNA
gctggatcctctagtgagctcacgatttccccattgccag

SEQ ID NO: 17 - DNA, 27 bp, synthetic construct
  source 1..27: mol_type = other DNA
ccaggcgttctcacaattccagatctg

SEQ ID NO: 18 - DNA, 55 bp, synthetic construct
  source 1..55: mol_type = other DNA
gagcggccccaagcttcaccgcggccgctcacttcttgtcgctgaacagctctgg

SEQ ID NO: 19 - DNA, 38 bp, synthetic construct
  source 1..38: mol_type = other DNA
agatcaaaggatcttcttgagatcctttttttctgcgc

SEQ ID NO: 20 - DNA, 43 bp, synthetic construct
  source 1..43: mol_type = other DNA
tttctacggggtctgattatttgttaactgttaattgtccttg

SEQ ID NO: 21 - DNA, 45 bp, synthetic construct
  source 1..45: mol_type = other DNA
aacaaataatcagaccccgtagaaacggccagcctcgcagagcag

SEQ ID NO: 22 - DNA, 52 bp, synthetic construct
  source 1..52: mol_type = other DNA
ggatctcaagaagatcctttgatctcttttccgctgcataaccctgcttcgg

SEQ ID NO: 23 - RNA, 20 nt, synthetic construct
  source 1..20: mol_type = other RNA
cattcgagtgggagcgttat

SEQ ID NO: 24 - DNA, 20 bp, synthetic construct
  source 1..20: mol_type = other DNA
ttctggttccactgggtttc

SEQ ID NO: 25 - DNA, 20 bp, synthetic construct
  source 1..20: mol_type = other DNA
cgaggtcattggcatgtaga

SEQ ID NO: 26 - DNA, 20 bp, synthetic construct
  source 1..20: mol_type = other DNA
cgaagtcatcctgctcaacc

SEQ ID NO: 27 - DNA, 21 bp, synthetic construct
  source 1..21: mol_type = other DNA
cgatacggtgtgccacaaacc

SEQ ID NO: 28 - RNA, 21 nt, synthetic construct
  source 1..21: mol_type = other RNA
gcgttcatccgtcatccactc

SEQ ID NO: 29 - DNA, 20 bp, synthetic construct
  source 1..20: mol_type = other DNA
tttgcaagcagcagattacg

SEQ ID NO: 30 - DNA, 48 bp, synthetic construct
  source 1..48: mol_type = other DNA
cgttgtaaaacgacggccagtgccaagcttgtgatgcacaacgccgac

SEQ ID NO: 31 - DNA, 39 bp, synthetic construct
  source 1..39: mol_type = other DNA
gctatgacatgattacgaattccatcaacgccgtcaccc

SEQ ID NO: 32 - DNA, 54 bp, synthetic construct
  source 1..54: mol_type = other DNA
tacactttatgcttccggctcgtatgttgtggtgatcagacctggaattgtgag

SEQ ID NO: 33 - DNA, 59 bp, synthetic construct
  source 1..59: mol_type = other DNA
cacaacatacgagccggaagcataaagtgtaaagcgtttattttgaagttttttgcgag

SEQ ID NO: 34 - DNA, 52 bp, synthetic construct
  source 1..52: mol_type = other DNA
aggaaacagctatgacatgattacgaattcctggtcattacccgcttccttg

SEQ ID NO: 35 - DNA, 55 bp, synthetic construct
  source 1..55: mol_type = other DNA
cgttgtaaaacgacggccagtgccaagcttctgtctatagggtgatgccgtaaca

SEQ ID NO: 36 - DNA, 21 bp, synthetic construct
  source 1..21: mol_type = other DNA
gggttccagcttgtccaatgg

SEQ ID NO: 37 - DNA, 22 bp, synthetic construct
  source 1..22: mol_type = other DNA
ccaacggtagaaggtaactgtg

SEQ ID NO: 38 - DNA, 24 bp, synthetic construct
  source 1..24: mol_type = other DNA
gaagatgtagctgctgttatactg

SEQ ID NO: 39 - DNA, 22 bp, synthetic construct
  source 1..22: mol_type = other DNA
actggccgtcgttttacaacgt

SEQ ID NO: 40 - DNA, 27 bp, synthetic construct
  source 1..27: mol_type = other DNA
gtcatagctgtttcctgtgtgaggcgt

SEQ ID NO: 41 - DNA, 21 bp, synthetic construct
  source 1..21: mol_type = other DNA
caacctgcaccatttctgcct

SEQ ID NO: 42 - DNA, 22 bp, synthetic construct
  source 1..22: mol_type = other DNA
gtgtacttgcgcccgatcatat

SEQ ID NO: 43 - DNA, 20 bp, synthetic construct
  source 1..20: mol_type = other DNA
ccgacaatcaacgcgagcgt

SEQ ID NO: 44 - DNA, 26 bp, synthetic construct
  source 1..26: mol_type = other DNA
tgcatatgatatctcctgtgtgactt

SEQ ID NO: 45 - DNA, 40 bp, synthetic construct
  source 1..40: mol_type = other DNA
acacaggaaacagctatgactgcccgttgtacctgctgtt

SEQ ID NO: 46 - DNA, 47 bp, synthetic construct
  source 1..47: mol_type = other DNA
gttgtaaaacgacggccagttggagcagcaaattgcagattgtgacg

SEQ ID NO: 47 - DNA, 40 bp, synthetic construct
  source 1..40: mol_type = other DNA
acacaggaaacagctatgaccttgcctctgccggaaaccc

SEQ ID NO: 48 - DNA, 40 bp, synthetic construct
  source 1..40: mol_type = other DNA
gttgtaaaacgacggccagtcctgccgcaaatcggtattc

SEQ ID NO: 49 - DNA, 19 bp, synthetic construct
  source 1..19: mol_type = other DNA
tttaccgcaaccctgagtg

SEQ ID NO: 50 - DNA, 22 bp, synthetic construct
  source 1..22: mol_type = other DNA
ggtcacgtagatcaccagaatg

SEQ ID NO: 51 - DNA, 20 bp, synthetic construct
  source 1..20: mol_type = other DNA
aggcattcgtgaagtcatgg

SEQ ID NO: 52 - DNA, 20 bp, synthetic construct
  source 1..20: mol_type = other DNA
atgtaaccgctgagaacgtc

SEQ ID NO: 53 - DNA, 40 bp, synthetic construct
  source 1..40: mol_type = other DNA
acacaggaaacagctatgacggcaccacttccacctggat

SEQ ID NO: 54 - DNA, 40 bp, synthetic construct
  source 1..40: mol_type = other DNA
gttgtaaaacgacggccagtagggtggcagtcgtctggat

SEQ ID NO: 55 - DNA, 23 bp, synthetic construct
  source 1..23: mol_type = other DNA
gcacaccgtcaaccattgagcct

SEQ ID NO: 56 - DNA, 40 bp, synthetic construct
  source 1..40: mol_type = other DNA
ctcaatggttgacggtgtgctcatgattgggcagtctcaa

SEQ ID NO: 57 - DNA, 38 bp, synthetic construct
  source 1..38: mol_type = other DNA
cttgtgttgctcccattcgcgattgccgggggcgcgtt

SEQ ID NO: 58 - DNA, 20 bp, synthetic construct
  source 1..20: mol_type = other DNA
gcgaatgggagcaacacaag

SEQ ID NO: 59 - DNA, 30 bp, synthetic construct
  source 1..30: mol_type = other DNA
gatatggatccatcaataaaacgaaaggct

SEQ ID NO: 60 - DNA, 27 bp, synthetic construct
  source 1..27: mol_type = other DNA
aattgttatccgctcacaattccaggt

SEQ ID NO: 61 - DNA, 45 bp, synthetic construct
  source 1..45: mol_type = other DNA
attgtgagcggataacaattctttgaacaaccgtgagtttgattg

SEQ ID NO: 62 - DNA, 40 bp, synthetic construct
  source 1..40: mol_type = other DNA
tttattgatggatccatatctcaggcgtcgaccagaatcg

SEQ ID NO: 63 - DNA, 42 bp, synthetic construct
  source 1..42: mol_type = other DNA
attgtgagcggataacaattcgtaggctaaaggccttgacac

SEQ ID NO: 64 - DNA, 40 bp, synthetic construct
  source 1..40: mol_type = other DNA
tttattgatggatccatatctcagccgttgaacacttcat

SEQ ID NO: 65 - DNA, 46 bp, synthetic construct
  source 1..46: mol_type = other DNA
aatcatcggctcgtataatgtgtggaattgtgagcggataacaatt

SEQ ID NO: 66 - DNA, 53 bp, synthetic construct
  source 1..53: mol_type = other DNA
cattatacgagccgatgattaattgtcaagcgtttattttgaagttttttgcg

SEQ ID NO: 67 - DNA, 25 bp, synthetic construct
  source 1..25: mol_type = other DNA
aattgttatccgctcacaattccac

SEQ ID NO: 68 - DNA, 42 bp, synthetic construct
  source 1..42: mol_type = other DNA
tttattgatggatccatatccccaatttttgtaacgccagct

SEQ ID NO: 69 - DNA, 20 bp, synthetic construct
  source 1..20: mol_type = other DNA
cgcaagcacctctcggcgat

SEQ ID NO: 70 - DNA, 20 bp, synthetic construct
  source 1..20: mol_type = other DNA
gccgcatagggattgacgat

SEQ ID NO: 71 - DNA, 40 bp, synthetic construct
  source 1..40: mol_type = other DNA
acacaggaaacagctatgactgctgaacctgccacacgat

SEQ ID NO: 72 - DNA, 21 bp, synthetic construct
  source 1..21: mol_type = other DNA
tacggtggtggcggcaacgct

SEQ ID NO: 73 - DNA, 41 bp, synthetic construct
  source 1..41: mol_type = other DNA
gcgttgccgccaccaccgtagaggccagtacaggtttacat

SEQ ID NO: 74 - DNA, 39 bp, synthetic construct
  source 1..39: mol_type = other DNA
gttgtaaaacgacggccagtcctggcggcaggcgcagac

SEQ ID NO: 75 - DNA, 41 bp, synthetic construct
  source 1..41: mol_type = other DNA
acacaggaaacagctatgacttgatacgtctgctgaagttc

SEQ ID NO: 76 - DNA, 21 bp, synthetic construct
  source 1..21: mol_type = other DNA
gatagctggcgttgagcggct

SEQ ID NO: 77 - DNA, 19 bp, synthetic construct
  source 1..19: mol_type = other DNA
ggtccaccggacctgcgtg

SEQ ID NO: 78 - DNA, 20 bp, synthetic construct
  source 1..20: mol_type = other DNA
cgtgctttcagccgcctggt

SEQ ID NO: 79 - DNA, 20 bp, synthetic construct
  source 1..20: mol_type = other DNA
gaagtgctgatcggcgacct

SEQ ID NO: 80 - DNA, 40 bp, synthetic construct
  source 1..40: mol_type = other DNA
attgtgagcggataacaattgagtgaccaggcgattgctg

SEQ ID NO: 81 - DNA, 39 bp, synthetic construct
  source 1..39: mol_type = other DNA
tttattgatggatccatatctcaaagctgggccacgatc

SEQ ID NO: 82 - DNA, 39 bp, synthetic construct
  source 1..39: mol_type = other DNA
acacaggaaacagctatgacctcttgtaatgcatcgcgt

SEQ ID NO: 83 - DNA, 20 bp, synthetic construct
  source 1..20: mol_type = other DNA
tggagggtgacggcatccct

SEQ ID NO: 84 - DNA, 40 bp, synthetic construct
  source 1..40: mol_type = other DNA
tttattgatggatccatatcttaacggggcggcaactgct

SEQ ID NO: 85 - DNA, 40 bp, synthetic construct
  source 1..40: mol_type = other DNA
attgtgagcggataacaattcccgggactattctcggcct

SEQ ID NO: 86 - DNA, 22 bp, synthetic construct
  source 1..22: mol_type = other DNA
ctacacctcccctttcgtattg

SEQ ID NO: 87 - DNA, 20 bp, synthetic construct
  source 1..20: mol_type = other DNA
tggtcgagcaggaaaatctg

SEQ ID NO: 88 - DNA, 4416 bp, synthetic construct
  source 1..4416: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcctcacacaggaaacagctatgacatgattacgaattctaatacaacaccacgttgatcccgacgaactgctggaaaatggacaacatcacgccaatcacgatcacccccacgccgaacatcagcaagcgcccgccggtcttgcggccgtggtccaaggagtgtttgatctcttgcacggcctgcgtggccagggtattgcccatgatcttccgcaagatgccttcggcttgttcctgcttgccacggctcatcagccagcgcgggctctcggggacggtgtacagcagcatcaggaacagcagcgctggaatacattcggacgcgaacatgtagcgccaaccatcggtattcagccagctggcatcgcccgaacgcgcaatgaagtaattcacgcagtacaccaacagctgcccaaagatgatcgcgaactggttgaagctgaccagcttcccccggatatgggcgggcgccaattcggcaatgtacatcggcgacaacatggaggccaggccgaccccgatcccaccgatgatgcggtagatcacgaactcgggcacgtaaccggccagatagactgggacggtgttgtccgggttaatcgaggtgaaacccagttccggccacgcggaccccacaccgctgataaaaaacaagacggccgcgatcttcaagctgtcgcggcgcccgaagcggttgctgcaatagccgcccagggccccgccgatgatgcagccgatcagggcgctcgccacgcagaagcccagcaggctgttggccgccgactccgacaggttctgcggggccacgaacacggtgttcaggctttccacggtgccgctgatcacggcggtatcataaccgaacagcaggccgcccagggtggccaccaaggtgatgctgaagatgtagctgctgttatactgggtgttcataagaaatctccttatgcttaagttgtcttcatgcttccacacaacatacgagccggaagcataaagtctaaggcctggaagacgcagacattccatactagtttatgccatgtcttgagaccttgacaattaatcatcggctcgtataatgtgtggaattgtgagcggataacaattggtctcacttaagtcacacaggagatatcatatgcaggcctacttcgaccagctggaccgcgtgcgctacgaaggcagcaaatcctcgaacccgctggccttccgtcactacaacccagacgagttggtgctgggtaagcgcatggaggagcatctgcgcttcgccgcctgctattggcacacgttctgctggaacggcgcggacatgttcggcgtcggcgcgtttaaccgcccctggcagcagccgggcgaggccctggcgctggcgaagcgcaaggcggacgtggccttcgagttcttccataaactgcacgtcccattctattgcttccatgacgtggacgtgagccaagcttggcactggccgtcgttttacaacgtcgtgaccggaattgccagctggggcgccctctggtaaggttgggaagccctgcaaacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataatcagaccccgtagaaaagatcaaaggatcttc

SEQ ID NO: 89 - DNA, 4410 bp, synthetic construct
  source 1..4410: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcctcacacaggaaacagctatgacatgattacgaattcagtgtcggcggcggctttcttgccgacgtaaccttggccacggcggtagaccttgaagcgggcacgcatgctgggcaggtgctcttcaaattcatcttcgacggtgttcggtggcaggcggtcgaggcgcacttcaccggtttggctaacccacagcaaatggtcgtcaaggctgtccttgcgcacggcgaacagctgggccaattggtcgatagtcggattgttgttcaagttcatcataaagcccccattacccattcataggtgattttcacagttggcgctatcacggtgtagcgcactaccaaggctgctacagcagcatcgcaacaggggctttctcacgctgccttttggacagtttccctctccacggacggcctgcgttaccgcgcaggccgtctggaacacccttgccaccgctcccgatcggggagacggcttcatcatgcccaagcgcgatgaacggcgtcaaccattttgtagtgaatatttttgctcactacattctgtcgttttgtccgacaatcaacgcgagcgttaggatccgacgtccataaaaaaatggcgccgatgggcgccatttttcactgttacagggtggcggtctgctgggtctttttggtttccggttcccacagcgcttccagttcttccaaggtcttacctttggtttcgggcacgaacttccacatgaacagggcggccagcacgcccatgcagccgtagatccagtagctgaagccgttgtggaagtgcgccaccaaccagctgttcttgtccatcattgggaaggtccaggacacgaagtagttggccagccactgggcggccacggcaatggccagggctttgccacggatggcattggggaagatttcggacagcaggacccagcacacgggaccccacgacatggcgaaggcggccacgtagaacaacatcgacagcagggcgacaatgcccggggcctgggtgtagaacgccgtgcccaagctgaacatgccgatggccatgcccagggcgccgatgatttgcagcggcttacggccgaatttatccacggtcatgatcgccaggaccgtaaacgtcaagttaatcacacccacgataatggtctgcagcagggcgatgtcggtcgaggcgcccagggtcttaaacacttctggcgcgtaatacaacaccacgttgatcccgacgaactgctggaaaatggacaacatcacgccaatcacgatcacccccacgccgaacatcagcaagcgcccgccggtcttgcggccgtggtccaaggagtgtttgatctcttgcacggcctgcgtggccagggtattgcccatgatcttccgcaagatgccttcggcttgttcctgcttgccacggctcatcagccagcgcgggctctcggggacggtgtaagcttggcactggccgtcgttttacaacgtcgtgaccggaattgccagctggggcgccctctggtaaggttgggaagccctgcaaacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataatcagaccccgtagaaaagatcaaaggatcttc

SEQ ID NO: 90 - DNA, 4659 bp, synthetic construct
  source 1..4659: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcctcacacaggaaacagctatgacatgattacgaattcaaccccagcagtcgctggaggctgccgcggtcaaagcccgtcaggccggtttcagcccgctgattctcggcgacctggaaggtgaatcgcgcgaagtggccaaggtgcatgccggtatcgcccggcaaatcgttcaacacggccagccgctcaaagcgccctgcgtgatcctgtcgggtggcgaaaccaccgtgaccgtgcgcggcaatggccgtggcgggcgtaacgccgagttcctgctcagcctcaccgaaagcctgaaaggcctgccgggcgtgtacgccctggccggtgacaccgacggcatcgatggctcggaagaaaacgccggtgccttcatgaccccggccagctacgccagcgccgaagccttgggcctgtcggccagcgacgagctggacaacaacaacggctacggctacttcgccgcgctggatgcgctgatcgtcaccgaaccgacccgcaccaacgtcaacgacttccgcgccatcctgatccttgagactgcccaatcatgagcacaccgtcaaccattgagccttgtggggcggatccccctcaagtcaaaagcctccggtcggaggcttttgactttctgctatggaggtcaggcctaggtgattattgtttctcgcaaaaaacttcaaaataaacgcttgacaattaatcatcggctcgtataatgtgtggaattgtgagcggataacaattcccgggactattctcggcctccagcgccgaggcgaggctcaacgttaccgcgcaggccgccggatggcgccgcgcccaagtacattgtggggatacatgcagacactaaaggtcgacctgggcgagcgtagctacccgatctacattggcgaaggcctgctggaccagcctgagctgctggcgccgcacattgcgggtcggcaggttgccatcgtctccaacgagaccgtcgcgccgctgtatctcgaacgcctgagcaaggcccttggtgcctactcggtactgcccgttgtgttgcccgatggtgaagcccacaagaactgggaaacattgcagctgatcttcgatggcctgctgacagctcggcatgatcgtcgtactactgtggtcgctctgggtggtggcgtgattggtgacatggctggtttcgcagccgcctgctaccagcgcggtgtcgatttcatccaggtgccgactaccctgttgtcccaggtcgactcttccgtgggtggcaagaccggcatcaatcatccgctgggcaagaacatggtcggtgcgttctatcagcccaaggcggtgctgatcgataccaccagcctcaagaccctgccagcgcgcgagctgtctgcaggtctggccgaagtgatcaagtacggcctgatttgcgacaagccgttcctcgcctggctcgaagacaatatgcaggcactaaagcttggcactggccgtcgttttacaacgtcgtgaccggaattgccagctggggcgccctctggtaaggttgggaagccctgcaaacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataatcagaccccgtagaaacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagagatcaaaggatcttc

SEQ ID NO: 91 - DNA, 7726 bp, synthetic construct
  source 1..7726: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttaggaattcgccacggtctacacctatgccatttccgatgtaccgggcgacctcgccacggtaatcgcctccggccccaccgtggccgacccgagcacctcggccgacgccctggccatcctcaaacgctacaacatcgaagcgcccaaagcggtcatcgactggctgaacaacccggcctcggaaaccgtcaaggccgatgacccggccctggcccgcagccacttccagttgatcgccaaaccccagcagtcgctggaggctgccgcggtcaaagcccgtcaggccggtttcagcccgctgattctcggcgacctggaaggtgaatcgcgcgaagtggccaaggtgcatgccggtatcgcccggcaaatcgttcaacacggccagccgctcaaagcgccctgcgtgatcctgtcgggtggcgaaaccaccgtgaccgtgcgcggcaatggccgtggcgggcgtaacgccgagttcctgctcagcctcaccgaaagcctgaaaggcctgccgggcgtgtacgccctggccggtgacaccgacggcatcgatggctcggaagaaaacgccggtgccttcatgaccccggccagctacgccagcgccgaagccttgggcctgtcggccagcgacgagctggacaacaacaacggctacggctacttcgccgcgctggatgcgctgatcgtcaccgaaccgacccgcaccaacgtcaacgacttccgcgccatcctgatccttgagactgcccaatcatgagcacaccgtcaaccattgagccttgtggggcggatccccctcaagtcaaaagcctccggtcggaggcttttgactttctgctatggaggtcaggcctaggtgattattgtttctcgcaaaaaacttcaaaataaacgctttacactttatgcttccggctcgtatgttgtggtgatcagacctggaattgtgagcggataacaattggtactttaaaaatagaaaatagcaaggagatatacatatgtcacaccccgcgttaacgcaactgcgtgcgctgcgctattttaaagagatccctgccctggatccgcaactgctcgactggctgttgctgcaggattccgtgacaaaacgttttgaacagcagggaaaaacggtaagcgtgacgatgatccgcgaagggtttgtcgagcagaatgaaatccccgaagaactgccgctgctgccgaaagagtctcgttactggttacgtgaaattttgttatgtgccgatggtgaaccgtggcttgccggtcgtaccgtcgttcctgtgtcaacgttaagcgggccggagctggcgttacaaaaattgggtaaaacgccgttaggacgctatctgttcacatcatcgacattaacccgggactttattgagataggccgtgatgccgggctgtgggggcgacgttcccgcctgcgattaagcggtaaaccgctgttgctaacagaactgtttttaccggcgtcaccgttgtactaagatatggatccatcaataaaacgaaaggctcagtcgaaagactgggcctttcgttttatctgttggccttgtgttgctcccattcgcgattgccgggggcgcgttgcgcccctttcgcgacacaaggccgctcccacagggccacgctggcacaagggaatgcagtccctgtgggagcggccttgtgtcgcgattggagggcaaagccctccctgaataccgctctacccgaccgcggaaaaccgaggcccaaagaggcccaccgctcccccatcaacctccactgactgccccatgtacaccaaaaatttcgtcaacccgtgccccgactgggccacggcgttactcaacggcttcagccaggtgctgctgctgcgcaaccccctgtgcggcctgtgctgcctgctggccatcctgctgaccgcacccaacctggtcggcggcgcgctgcttggcgccctggccggcctgctcaccgcccaggcacgcggctatgaccgtgccgaccgccaggccgggctttactgctacaacggcgtactgatcggcatcctgatcagcgccgtgctgccctggtcggccatattgccccccctgatcatcgccgccggcggcttgtccagcatcatcacgcaccagtggcgcaagcgtggcggcaagttgctgattgcctacacctcccctttcgtattgctgggctgggccacactgctggtcgccagcccctcgcccagcgggttcgtcgaagccgacccgctgtatgcgttggcccgcggcgtgggccagattttcctgctcgaccagcccttggccgggttgctgatcatcgtcggcatgggatccctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttc